(19) United States
(12) Patent Application Publication
(10) Pub. No.: US 2021/0171503 A1
(43) Pub. Date: Jun. 10, 2021

(54) GLYCOLATE OXIDASE INHIBITORS AND USE THEREOF

(71) Applicant: ORFAN BIOTECH INC., Palo Alto, CA (US)

(72) Inventors: Hans MAAG, Kleines Wiesental (DE); Miguel Xavier Fernandes, San Cristobal de La Laguna (ES)

(21) Appl. No.: 16/958,348

(22) PCT Filed: Dec. 28, 2018
(86) PCT No.: PCT/US2018/067863

§ 371 Date: Jun. 26, 2020

Related U.S. Application Data
(60) Provisional application No. 62/612,161, filed on Dec. 29, 2017.

(51) Int. Cl. C07D 403/12; C07D 285/06; C07D 417/12; C07D 231/38; C07D 401/12; C07D 413/12; C07D 261/18; C07D 275/03; C07D 237/24

(52) U.S. Cl. CPC C07D 403/12; C07D 285/06; C07D 417/12; C07D 231/38; C07D 237/24; C07D 413/12; C07D 261/18; C07D 275/03; C07D 401/12

(57) ABSTRACT

The present invention provides pyrazoles, isoxazoles, isothiazoles, thiadiazoles, and pyridazines according to Formula I as described herein, and pharmaceutically acceptable salts thereof. Pharmaceutical compositions and methods for treating primary hyperoxaluria, type I (PH) and kidney stones are also described.

### CROSS-REFERENCES TO RELATED APPLICATIONS

This application is the U.S. National Stage Entry under § 371 of International Application No. PCT/US2018/067863, filed Dec. 28, 2018, which claims priority to U.S. Provisional Pat. Appl. No. 62/612,161, filed on Dec. 29, 2017, which each application is incorporated herein by reference in its entirety.

### BACKGROUND OF THE INVENTION

Kidney stone disease (KSD) has a prevalence of approximately 10% in developed countries with lifetime recurrence rates of up to 50% [Johri, et al. (2010) Nephron Clin Pract. 116: c159]. KSD patients present with hematuria and renal colic and medical treatment is essentially symptomatic. The administration of drugs to facilitate stone passage is effective for small stones (<5 mm). For bigger stones, extracorporeal sound waves or minimally invasive surgery are used to break the stone into small pieces that can more easily pass the urinary tract [Coe et al. (2005) J. Clin. Invest. 115: 2598].

Approximately 75% of kidney stones contain primarily calcium oxalate and elevated levels of urinary oxalate are found in up to 50% of KSD patients. Furthermore, increased levels of urinary oxalate increase the risk of forming kidney stones [Moe (2006) Lancet 367: 333; Sakhaee (2009) Kidney Int. 75: 585; Kaufman et al. (2008) J Am Soc Nephrol. 19: 1197]. In mammals, calcium has vital physiological roles in so many processes that its levels are tightly regulated. Oxalate, however, is a metabolic end-product with no known physiological role. Oxalate is a divalent anion that must be eliminated with the urine and tends to precipitate as tissue-damaging insoluble calcium oxalate crystals.

Primary hyperoxalurias (PH) are a group of rare metabolic diseases, with autosomal recessive inheritance, affecting the glyoxylate or the hydroxyproline pathways. All of them have in common an overproduction of oxalate. So far, three forms of primary hyperoxaluria have been identified. They are referred as primary hyperoxaluria types 1, 2, and 3. Primary hyperoxaluria type 1 (PH1) is caused by mutation of liver-specific enzyme alanine:glyoxylate aminotransferase (AGT). Primary hyperoxaluria type 2 (PH2) is caused by mutation of glyoxylate reductase-hydroxypyruvate reductase (GRHPR). Primary hyperoxaluria type 3 (PH3) is caused by mutation of 4-hydroxy-2-oxoglutarate aldolase (HOGA1). PH1 eventually leads to renal failure after several years. PH2 and PH3 have a less severe course. Approximately 80% of PH patients suffer PH1, the most severe PH type. Considering its statistical predominance, most studies on PH essentially refer to PH1 [Salido et al. (2012) Biochim Biophys Acta. 1822: 1453].

Since calcium levels are so tightly regulated in the organism, changing them in urine is extremely difficult, and it may also produce undesired effects in vital physiological processes. Minor increases in urinary oxalate can produce large effect on calcium oxalate crystal formation, and elevated levels of urinary oxalate are a major risk factor for the formation of calcium oxalate kidney stones [Pak, et al. (2004)Kidney Int. 66: 2032]. Consequently, a small decrease in oxalate concentration could lower the calcium oxalate level below saturation, and thus prevent calcium oxalate stone formation. Irrespective of the urinary oxalate levels in individuals with kidney stone disease, primary hyperoxaluria, or secondary hyperoxaluria, lowering UOx levels will decrease the contribution of oxalate to calcium oxalate formation, and thus lower the probability of stone formation and/or alleviate the severity of excessive calcium oxalate deposition related conditions [Marengo et al. (2008) Nat Clin Pract Nephrol. 4: 368].

The development of an effective drug that reduces urinary oxalate levels can be a valuable therapeutic option in the prophylaxis and treatment of conditions related to calcium oxalate. Common approaches for treatment of urolithiasis due to calcium oxalate include surgical removal of stones, dietary changes increase fluid intake and to restrict oxalate intake, urine alkalization, diuretics, and crystallization inhibitors such as citrate, bicarbonate, and magnesium [Moe, supra]. However, none of these therapeutic approaches tackles the origin of the conditions. No drug which specifically inhibits the endogenous biosynthetic formation of oxalate is commercially available for the prophylaxis and treatment of calcium oxalate deposition related conditions.

In humans, dietary oxalate contributes only 10-50% to the amount of excreted urinary oxalate [Holmes, et al. (2001) Kidney Int. 59: 270]. Most urinary oxalate is derived from the endogenous metabolism, mainly in liver. In humans, the major precursor of oxalate is glyoxylate. Therefore, approaches to reduce the production of oxalate must block the conversion of glyoxylate into oxalate, or block the production of glyoxylate from its precursors. In humans, the major precursor of glyoxylate is glycolate in a reaction catalyzed by the peroxisomal liver enzyme glycolate oxidase (GO), also termed hydroxyacid oxidase 1. Pharmacological inhibition of GO activity with small molecules will diminish endogenous oxalate production and lead to a reduction of calcium oxalate levels in the urine, thus providing a specific approach for prophylaxis and treatment of calcium oxalate deposition and related conditions. There is evidence that GO is a safe therapeutic target in humans. A report describes a finding where a defective splice variant of human GO in an individual simply causes isolated asymptomatic glycolic aciduria with no apparent ill effects [Frishberg, et al. (2014) J Med Genet. 51: 526].

There is need in the art for effective therapeutic approaches that inhibit biosynthetic formation of oxalate and for treating PH1 and other conditions related to deposition of calcium oxalate. The present invention meets these and other needs.

### BRIEF SUMMARY OF THE INVENTION

In one embodiment, the invention provides compounds according to Formula I as described herein, and pharmaceutically acceptable salts thereof.

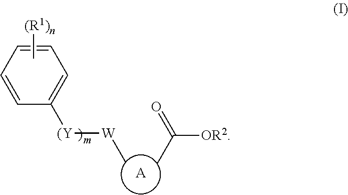

In the compounds of Formula I:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₁₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₁₋₆ alkenylene, C₁₋₆ alkynylene, and 2- to 6-membered heteroalkylene; and
each heterocyclyl is optionally and independently substituted with one or more amine protecting groups.

In another embodiment, the invention provides a pharmaceutical composition containing a compound as described herein and a pharmaceutically acceptable excipient.

In another embodiment, the invention provides a method for treating primary hyperoxaluria, type I (PH1). The method includes administering to a subject in need thereof a therapeutically effective amount of a compound described herein.

In other embodiment, the invention provides a method for treating kidney stones. The method includes administering to a subject in need thereof a therapeutically effective amount of a compound described herein.

### BRIEF DESCRIPTION OF THE DRAWINGS

FIG. 1 shows the catalytic reactions used for the glycolate oxidase (GO) enzymatic assay described herein. TOP: GO enzymatic conversion of glycolate to glyoxylate, with the concomitant reduction of the cofactor flavin mononucleotide (FMN), which uses molecular oxygen (O₂) for recovering its oxidative state, releasing hydrogen peroxide (H₂O₂). BOTTOM: Trinder reaction in which horseradish peroxidase (HRP) uses hydrogen peroxide, 4-aminoantipyrine and a phenol derivative (sulfonated DCIP) to generate a quinoneimine dye that is spectrophotometrically measured.

FIG. 2 shows the catalytic reactions used in the oxalate determination assay described herein. Oxalate oxidase transforms oxalate and molecular oxygen (O₂) in two molecules of carbon dioxide (CO₂) and hydrogen peroxide (H₂O₂). H₂O₂, 3-methyl-2-benzothiazolinone hydrazone (MBTH) and 3-(dimethylamino) benzoic acid (DMAB) react with horseradish peroxidase (HRP) to give an indamine dye and water.

FIG. 3 shows Formula I according to the present disclosure.

### DETAILED DESCRIPTION OF THE INVENTION

### I. Introduction

Metabolic diseases are caused by mutations in genes that code key enzymes of a metabolic pathway. Generally, this results in a failure to metabolize a compound with its subsequent harmful build-up. In the case of primary hyperoxaluria type I (PHI), mutation of alanine-glyoxylate transaminated (AGT) disrupts the glyoxylate detoxification pathway. Mutation of AGT prevents AGT from converting glyoxylate to pyruvate, and the resulting build-up of glyoxylate results in higher levels of oxalate and oxalate-containing kidney stones. Glycolate oxidase (GO) is a peroxisomal hepatic enzyme which catalyzes the oxidation of glycolate to glyoxylate, the AGT substrate. As such, GO plays a pivotal role in glyoxylate production while AGT plays a pivotal role in glyoxylate detoxification. The present invention provides compounds and methods for treating PHI by targeting GO, the source of the AGT substrate. GO inhibitors according to the invention reduce glyoxylate levels in PHI patients, thus compensating for the inability of mutant AGT-located downstream of GO in the glyoxylate detoxification pathway—to metabolize glyoxylate and preventing the harmful build-up of oxalate.

### II. Definitions

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, alkylamino, amido, acyl, nitro, cyano, and alkoxy.

As used herein, the term “alkylene” refers to an alkyl group, as defined above, linking at least two other groups (i.e., a divalent alkyl radical). The two moieties linked to the alkylene group can be linked to the same carbon atom or different carbon atoms of the alkylene group.

As used herein, the term “alkoxy,” by itself or as part of another substituent, refers to a moiety having the formula —OR, wherein R is an alkyl group as defined herein. Examples of alkoxy groups include, but are not limited to, methoxy, ethoxy, and isopropyloxy.

As used herein, the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃-s cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. “Substituted cycloalkyl” groups can be substituted with one or more groups selected from halo, hydroxy, amino, alkylamino, amido, acyl, nitro, cyano, and alkoxy. The term “lower cycloalkyl” refers to a cycloalkyl radical having from three to seven carbons including, for example, cyclopropyl, cyclobutyl, cyclopentyl, cyclohexyl, and cycloheptyl.

As used herein, the term “heteroalkyl,” by itself or as part of another substituent, refers to an alkyl group of any suitable length and having from 1 to 3 heteroatoms such as N, O and S. For example, heteroalkyl can include ethers, thioethers and alkyl-amines. Additional heteroatoms can also be useful, including, but not limited to, B, Al, Si and P. The heteroatoms can be oxidized to form moieties such as, but not limited to, —S(O)— and —S(O)₂—. The heteroatom portion of the heteroalkyl can replace a hydrogen of the alkyl group to form a hydroxy, thio, or amino group. Alternatively, the heteroatom portion can be the connecting atom, or be inserted between two carbon atoms.

As used herein, the term “heteroalkylene” refers to a heteroalkyl group, as defined above, linking at least two other groups (i.e., a divalent heteroalkyl radical). The two moieties linked to the heteroalkylene group can be linked to the same atom or different atoms of the heteroalkylene group.

As used herein the term “heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclic groups can be saturated (e.g., azetidinyl, pyrrolidinyl, piperidinyl, morpholine, oxetanyl, tetrahydrofuranyl, or tetrahydropyranyl) or unsaturated (e.g., 2,3-dihydrofuranyl, 2,5-dihydrofuranyl, 3,4-dihydropyranyl, 3,6-dihydropyranyl, or 1,4-dihydropyridinyl). Heterocyclyl groups can be unsubstituted or substituted. “Substituted heterocyclyl” groups can be substituted with one or more groups selected from halo, hydroxy, amino, oxo (═O), alkylamino, amido, acyl, nitro, cyano, and alkoxy.

The heterocyclyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocyclyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocyclyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein, the term “aryl,” by itself or as part of another substituent, refers to an aromatic ring system having any suitable number of ring atoms and any suitable number of rings. Aryl groups can include any suitable number of ring atoms, such as 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. “Substituted aryl” groups can be substituted with one or more groups selected from halo, hydroxy, amino, alkylamino, amido, acyl, nitro, cyano, and alkoxy.

As used herein, the term “arylalkyl” refers to an aryl group that is bonded to a compound via an alkylene group as described herein. Examples of arylalkyl groups include, but are not limited to, benzyl and phenethyl.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

As used herein, the terms “halo” and “halogen,” by themselves or as part of another substituent, refer to a fluorine, chlorine, bromine, or iodine atom.

As used herein, the term “cyano” refers to a carbon atom triple-bonded to a nitrogen atom (i.e., the moiety —C≡N).

As used herein, the term “1,2,3-thiadiazol-4,5-diyl” refers to a divalent radical having the structure shown below, wherein the wavy line is the point of connection to the —W— moiety in Formula I disclosed herein and the dashed line is the point of connection to the —C(O)OR² moiety in Formula I disclosed herein:

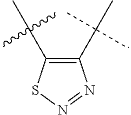

As used herein, the term “(5-methyl)-1H-pyrazol-3,4-diyl” refers to a divalent radical having the structure shown below, wherein the wavy line is the point of connection to the —W— moiety in Formula I disclosed herein and the dashed line is the point of connection to the —C(O)OR² moiety in Formula I disclosed herein:

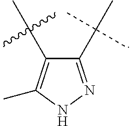

As used herein, the term “(5-methyl)-isoxazol-3,4-diyl” refers to a divalent radical having the structure shown below, wherein the wavy line is the point of connection to the —W— moiety in Formula I disclosed herein and the dashed line is the point of connection to the —C(O)OR² moiety in Formula I disclosed herein:

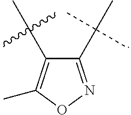

As used herein, the term “(5-methyl)-isothiazol-3,4-diyl” refers to a divalent radical having the structure shown below, wherein the wavy line is the point of connection to the —W— moiety in Formula I disclosed herein and the dashed line is the point of connection to the —C(O)OR² moiety in Formula I disclosed herein:

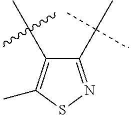

As used herein, the term “pyridazin-3,4-diyl” refers to a divalent radical having the structure shown below, wherein the wavy line is the point of connection to the —W— moiety in Formula I disclosed herein and the dashed line is the point of connection to the —C(O)OR² moiety in Formula I disclosed herein:

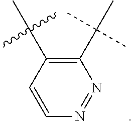

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino” refers to a moiety —NR₃, wherein each R group is H or alkyl. An amino moiety can be ionized to form the corresponding ammonium cation.

As used herein, the term “hydroxy” refers to the moiety —OH.

As used herein, the term “carboxy” refers to the moiety —C(O)OH. A carboxy moiety can be ionized to form the corresponding carboxylate anion.

As used herein, the term “amido” refers to a moiety —NRC(O)R or —C(O)NR₂, wherein each R group is H or alkyl.

As used herein, the term “nitro” refers to the moiety —NO₂.

As used herein, the term “oxo” refers to an oxygen atom that is double-bonded to a compound (i.e., O═).

As used herein, the term “amine protecting group” refers to a chemical moiety that renders an amino group unreactive, but is also removable so as to restore the amino group. Examples of amine protecting groups include, but are not limited to, benzyloxycarbonyl; 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; and the like. Other amine protecting groups are known to those of skill in the art including, for example, those described by Green and Wuts (Protective Groups in Organic Synthesis, 4^{th} Ed. 2007, Wiley-Interscience, New York).

As used herein, the term “salt” refers to acid or base salts of the compounds of the invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid, fumaric acid, and the like) salts, quaternary ammonium (methyl iodide, ethyl iodide, and the like) salts. It is understood that the pharmaceutically acceptable salts are non-toxic.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

As used herein, the term “excipient” refers to a substance that aids the administration of an active agent to a subject. By “pharmaceutically acceptable,” it is meant that the excipient is compatible with the other ingredients of the formulation and is not deleterious to the recipient thereof. Pharmaceutical excipients useful in the present invention include, but are not limited to, binders, fillers, disintegrants, lubricants, glidants, coatings, sweeteners, flavors and colors.

As used herein, the terms “treat,” “treatment,” and “treating” refer to any indicia of success in the treatment or amelioration of an injury, pathology, condition, or symptom (e.g., cognitive impairment), including any objective or subjective parameter such as abatement; remission; diminishing of symptoms or making the symptom, injury, pathology or condition more tolerable to the patient; reduction in the rate of symptom progression; decreasing the frequency or duration of the symptom or condition. In some situations, treating can including preventing the onset of the injury, pathology, condition, or symptom. The treatment or amelioration of symptoms can be based on any objective or subjective parameter; including, e.g., the result of a physical examination.

As used herein, the terms “primary hyperoxaluria, type I” and “PH1” are interchangeable and refer to a condition caused by the deficiency of alanine:glyoxylate aminotransferase (AGT), a liver enzyme. This deficiency causes impaired glyoxylate metabolism in the liver and an ultimate increase in oxalate synthesis, contributing to the formation of calcium oxalate kidney stones.

As used herein, the term “kidney stone” refers to a small, solid particle that occur in the kidneys, renal pelvis, ureter, urinary bladder, and/or urethra. Commonly, kidney stones contain or consist of calcium salt particles including, but not limited to, calcium oxalate particles and calcium phosphate particles (e.g., apatite particles or brushite particles). Kidney stones can also contain or consist of uric acid, struvite (i.e., NH₄MgPO₄.6H₂O particles), and cystine (i.e., particles containing oxidized cysteine disulfide dimer). Kidney stones typically range in size from less than a millimeter in their largest dimension to 5 or more centimeters in their largest dimension. Kidney stones often form in the kidney or renal pelvis and, when they are small enough (e.g., less than 5 mm), they can pass through the ureter, bladder, and urethra to be eliminated from the body via urination. Kidney stones often cause severe pain in the side and back, below the ribs, and severe pain in the lower abdomen and groin. Other symptoms of kidney stones include, but are not limited to, pain upon urination, abnormally colored urine (e.g., pink, red, or brown), cloudy urine, foul-smelling urine, nausea and vomiting, a persistent need to urinate, low urine volume, fever, and chills. The presence of kidney stones in the urinary system can be confirmed using imaging techniques such as abdominal X-ray, CT scan, and ultrasound.

The terms “glycolate oxidase” and “GO” are used interchangeably to refer to the liver peroxisomal enzyme glycolate oxidase 1 (GO1), also known as hydroxyacid oxidase 1 (HAO1). The human enzyme is cataloged under NCBI Accession No. NP_060015.1 and UniProtKB Reference No. Q9UJM8. The mouse enzyme is cataloged under GenBank Accession No. EDL28373.1 and UniProtKB Reference No. Q9WU19. The enzyme catalyzes the conversion of glycolic acid to glyoxylic acid, an oxalic acid precursor.

As used herein, the term “administering” refers to oral, topical, parenteral, intravenous, intraperitoneal, intramuscular, intralesional, intranasal, subcutaneous, or intrathecal administration to a subject, as well administration as a suppository or the implantation of a slow-release device, e.g., a mini-osmotic pump, in the subject.

As used herein, the term “subject” refers to a person or other animal to whom a compound or composition as described herein is administered. In some embodiments, the subject is human. In some embodiments, the subject is a human having a mutation in the AGXT gene encoding alanine-glyoxylate amino transferase (AGT).

As used herein, the terms “effective amount” and “therapeutically effective amount” refer to a dose of a compound such as a glycolate oxidation inhibitor that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11th Edition, 2006, Brunton, Ed., McGraw-Hill; and Remington: The Science and Practice of Pharmacy, 21^{st} Edition, 2005, Hendrickson, Ed., Lippincott, Williams & Wilkins).

### III. Glycolate Oxidase Inhibitors and Related Compounds

The invention provides compounds for inhibiting glycolate oxidase activity, making them useful in the treatment of PH1 and kidney stones. The compounds can be conveniently prepared from commercially available starting materials and common intermediates, as described in more detail below. Accordingly, a first embodiment of the invention provides compound according to Formula I

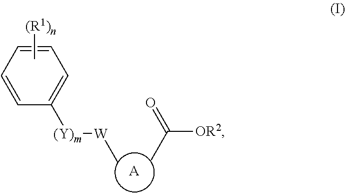

or a pharmaceutically acceptable salt or C₁₋₆ alkyl ester thereof,

wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₁₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₁₋₆ alkenylene, C₁₋₆ alkynylene, and 2- to 6-membered heteroalkylene; and
each heterocyclyl is optionally and independently substituted with one or more amine protecting groups;
provided that if ring A is (5-methyl)-isoxazol-3,4-diyl, W is —CH₂—, subscript n is 1, and subscript m is 1, then Y is 0;
provided that if ring A is (5-methyl)-isoxazol-3,4-diyl, W is —CH₂—, and subscript m is 0, then subscript n is 1, 2, or 3;
provided that if ring A is (5-methyl)-isoxazol-3,4-diyl, W is —CH₂—, subscript n is 1, and subscript m is 0, then R is other than 3-bromo, 4-bromo, 3-chloro, 4-chloro, 3-fluoro, and 4-fluoro;
provided that if ring A is (5-methyl)-1H-pyrazol-3,4-diyl, W is —NR³—, R³ is H or C₁₋₆ alkyl, and subscript m is 0, then subscript n is 1, 2, or 3;
provided that if ring A is (5-methyl)-1H-pyrazol-3,4-diyl, W is —NR³—, R³ is H or C₁₋₆ alkyl, subscript m is 1, and Y is —CH₂—, then subscript n is 1, 2, or 3;
provided that if ring A is (5-methyl)-isoxazol-3,4-diyl, W is —NH—, subscript n is 1, and subscript m is 0, then R is other than 3-cyano, 4-cyano, 3-bromo, 4-bromo, 3-chloro, 4-chloro, 3, fluoro, and 4-fluoro;
provided that if ring A is (5-methyl)-1H-pyrazol-3,4-diyl, W is —CH₂—, subscript n is 1, and subscript m is 1, then Y is 0;
provided that if ring A is (5-methyl)-1H-pyrazol-3,4-diyl, W is —CH₂—, and subscript m is 0, then subscript n is 1, 2, or 3;
provided that if ring A is (5-methyl)-1H-pyrazol-3,4-diyl, W is —CH₂—, subscript n is 1, and subscript m is 0, then R is other than 3-cyano, 4-cyano, 4-bromo, 3-chloro, 4-chloro, 3-fluoro, 4-fluoro, 3-pyridin-3-yl, 3-pyridin-4-yl, 3-(4-cyanophenyl), 3-(4-fluorophenyl), 4-(4-fluorophenyl), 3-phenoxyphenyl, and 4-phenoxyphenyl;
provided that if ring A is 1,2,3-thiadiazol-4,5-diyl, W is —CH₂— or —NH—, and subscript m is 0, then subscript n is 1, 2, or 3;
provided that if ring A is 1,2,3-thiadiazol-4,5-diyl, W is —S—, Y is —CH₂—, and subscript m is 1, then subscript n is 1, 2, or 3;
provided that if ring A is 1,2,3-thiadiazol-4,5-diyl, W is S, subscript n is 1, and subscript m is 0, then R¹ is other than 4-chloro;
provided that if ring A is 1,2,3-thiadiazol-4,5-diyl, W is —NR³—, Y is —CHR⁴—, R³ is butyl, R⁴ is H, subscript n is 1, and subscript m is 1, then R¹ is other than 4-(2H-tetrazol-5-yl)phenyl;
provided that if ring A is pyridazin-3,4-diyl, W is —NR³—, Y is —CHR⁴—, R³ is propyl, R⁴ is H, subscript n is 1, and subscript m is 1, then R is other than 4-(2H-tetrazol-5-yl)phenyl.

In some embodiments:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0 or 1;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —CHR⁴—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a};
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, and 2- to 6-membered heteroalkylene; and
each heterocyclyl is optionally and independently substituted with one or more amine protecting groups.

In some embodiments, the invention provides a 1,2,3-thiadiazole compound, or a pharmaceutically acceptable salt thereof, according to Formula II:

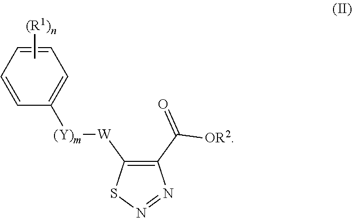

In some embodiments, the invention provides a compound, or a pharmaceutically acceptable salt thereof, according to Formula III:

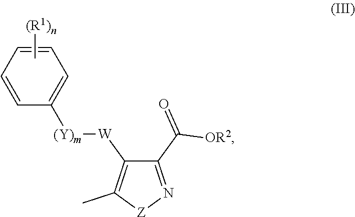

wherein Z is selected from the group consisting of NH, O, and S.

In some embodiments, the invention provides a (5-methyl)-1H-pyrazole compound, or a pharmaceutically acceptable salt thereof, according to Formula IIIa:

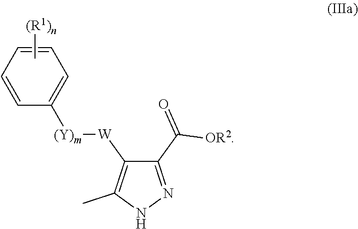

In some embodiments, the invention provides a (5-methyl)-isoxazole compound, or a pharmaceutically acceptable salt thereof, according to Formula IIIb:

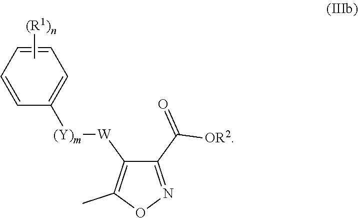

In some embodiments, the invention provides a (5-methyl)-isothiazole compound, or a pharmaceutically acceptable salt thereof, according to Formula IIIc:

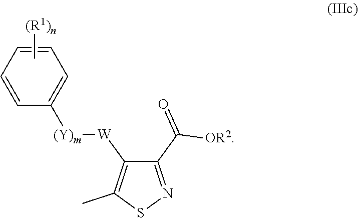

In some embodiments, the invention provides a pyridazine compound, or a pharmaceutically acceptable salt thereof, according to Formula IV:

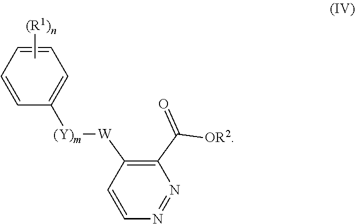

In some embodiments, the invention provides compounds of Formula I, Formula II, Formula III, Formula IIIa, Formula IIIb, Formula IIIc, or Formula IV, wherein subscript n is 0 or 1.

In some embodiments, the invention provides compounds of Formula I, Formula II, Formula III, Formula IIIa, Formula IIIb, Formula IIIc, or Formula IV, wherein —(Y)ₘ—W— is other than —CH₂—CH₂—.

In some embodiments, W is —CHR³— in compounds of Formula I, Formula II, Formula III, Formula IIIa, Formula IIIb, Formula IIIc, or Formula IV. In some embodiments, W is —CHR³— and R³ is H (i.e., W is —CH₂—). In some embodiments, W is —CHR³— and R³ is C₁₋₆ alkyl. For example, R³ can be methyl, ethyl, n-propyl, isopropyl, n-butyl, sec-butyl, t-butyl, n-pentyl, or n-hexyl. In some embodiments, W is —CHR³— and R³ is C₇₋₁₆ arylalkyl. For example, R³ can be benzyl, phenethyl, or another arylalkyl group. In some such embodiments, the aryl moiety is substituted with R^{3a}. In some embodiments, R^{3a} is halo. In some embodiments, R^{3a} is -M-(C₆₋₁₀ aryl). In some embodiments, R^{3a} is -M-(C₆₋₁₀ aryl), M is a single bond, and C₆₋₁₀ aryl is substituted with R^{3b}.

In some embodiments, including the embodiments described above wherein W is —CHR³—, subscript m is 0. In some embodiments, including the embodiments described above wherein W is —CHR³—, subscript m is 1 and Y is —O—. In some embodiments, subscript n is 1, R is selected from the group consisting of halo and -L-aryl, L is selected from the group consisting of a bond and —O—, and aryl is optionally substituted with R^{1a}. In some embodiments, subscript n is 1, R is -L-aryl, and L is selected from the group consisting of a bond and —O—. In some embodiments, subscript n is 1 and R¹ is phenoxy. In some embodiments, subscript n is 1 and R is 4-cyanophenyl.

In some embodiments, W is selected from the group consisting of —S—, —S(O)—, and —S(O)₂— in compounds of Formula I, Formula II, Formula III, Formula IIIa, Formula IIIb, Formula IIIc, or Formula IV. In some such embodiments, W is —S—. In some embodiments, W is —S— and subscript m is 0. In some embodiments, W is —S(O)— and subscript m is 0. In some embodiments, W is —S(O)₂— and subscript m is 0.

In some embodiments, W is —S—, subscript m is 1, and Y is —CHR⁴—. In some embodiments, W is —S—, subscript m is 1, Y is —CHR⁴—, and R⁴ is C₁₋₆ alkyl. For example, R⁴ can be methyl, ethyl, n-propyl, isopropyl, n-butyl, sec-butyl, t-butyl, n-pentyl, or n-hexyl. In some embodiments, W is —S—, subscript m is 1, Y is —CHR⁴—, and R⁴ is C₇₋₁₆ arylalkyl. For example, R⁴ can be benzyl, phenethyl, or another arylalkyl group. In some such embodiments, the aryl moiety is substituted with R^{4a}. In some embodiments, R^{4a} is halo. In some embodiments, R^{4a} is -Q-(C₆₋₁₀ aryl). In some embodiments, R^{4a} is -Q-(C₆₋₁₀ aryl), Q is a single bond, and C₆₋₁₀ aryl is substituted with R^{4b}. In some embodiments, including the embodiments described above wherein W is —S—, subscript n is 0. In some embodiments, including the embodiments described above wherein W is —S—, subscript n is 1. In some embodiments, including the embodiments described above wherein W is —S—, subscript n is 1, and R¹ is halo.

In some embodiments, W is —NR³— in compounds of Formula I, Formula II, Formula III, Formula IIIa, Formula IIIb, Formula IIIc, or Formula IV. In some embodiments, W is —NR³—, and subscript m is 0. In some embodiments, W is —NR³—, subscript m is 0, subscript n is 1, and R¹ is selected from the group consisting of halo and cyano. In some embodiments, R¹ is fluoro (e.g., 3-fluoro or 4-fluoro). In some embodiments, R¹ is bromo (e.g., 3-bromo or 4-bromo).

In some embodiments, W is —NR³—, subscript m is 0, subscript n is 1, and R¹ is selected from the group consisting of heteroaryl and L-aryl, wherein aryl is substituted with R^{1a}. In some such embodiments, R^{1a} is selected from the group consisting of halo, cyano, and -M-heterocyclyl. In some embodiments, heterocyclyl is selected from the group consisting of azetidinyl, piperidinyl, piperazinyl, and morpholino. In some embodiments, heterocyclyl is substituted with benzyloxycarbonyl, 9-fluorenylmethyloxycarbonyl, and/or tert-butyloxycarbonyl. In some embodiments, L is a single bond or —O—. In some embodiments, L is C₁₋₆ alkylene (e.g., methylene or methanediyl; ethylene or ethane-1,2-diyl; n-propylene or propane-1,3-diyl; and the like) or M is 2- to 6-membered heteroalkylene (e.g., —CH₂CH₂—O—, —(CH₂)₃CH₂—O—, and the like).

In some embodiments, including any of the embodiments described above wherein W is —NR³—, R³ is H. In some embodiments, including any of the embodiments described above wherein W is —NR³—, R³ is C₁₋₆ alkyl. For example, R³ can be methyl, ethyl, n-propyl, isopropyl, n-butyl, sec-butyl, t-butyl, n-pentyl, or n-hexyl. In some embodiments, including any of the embodiments described above wherein W is —NR³—, R³ is C₇₋₁₆ arylalkyl. For example, R³ can be benzyl, phenethyl, or another arylalkyl group. In some such embodiments, the aryl moiety is substituted with R^{3a}. In some embodiments, R^{3a} is halo. In some embodiments, R^{3a} is -M-(C₆₋₁₀ aryl). In some embodiments, R^{3a} is -M-(C₆₋₁₀ aryl), M is a single bond, and C₆₋₁₀ aryl is substituted with R^{3b}.

In some embodiments, including any of the embodiments described above wherein W is —NR³—, subscript m is 0. In some embodiments, including any of the embodiments described above wherein W is —NR³—, subscript m is 1 or 2. In some embodiments, subscript n is 1, subscript m is 1 or 2, W is —NR³—, each Y is —CHR⁴—. In some embodiments, subscript n is 1, subscript m is 1 or 2, W is —NR³—, each Y is —CHR⁴—, R¹ is halo, and R³ is C₇₋₁₆ arylalkyl substituted with R^{3a}. In some such embodiments, R^{3a} is halo.

In some embodiments: subscript n is 1, R¹ is selected from the group consisting of halo, cyano, and -L-(C₆₋₁₀ aryl), wherein aryl is optionally substituted with R^{1a}, and W is —NR³—. In some embodiments, L is selected from the group consisting of a bond, —O—, and C₁₋₆ alkylene (e.g., methylene or methanediyl; ethylene or ethane-1,2-diyl; n-propylene or propane-1,3-diyl; and the like). In some embodiments, R^{1a} is selected from the group consisting of halo, and cyano. In some such embodiments, subscript m is 1 or 2, and each Y is —CHR⁴—. In some such embodiments, R⁴ is H.

In some embodiments: subscript n is 1; R¹ is selected from the group consisting of halo, cyano, and -L-(C₆₋₁₀ aryl), wherein aryl is optionally substituted with R^{1a}; R^{1a} is selected from the group consisting of halo and cyano; L is selected from the group consisting of a bond, —O—, and C₁₋₆ alkylene; W is —NR³—; R³ is H; m is 1 or 2; and Y is —CHR⁴—. In some such embodiments, each R⁴ is H.

In some embodiments:
subscript n is 1;
subscript m is 0, 1, or 2;
W is —NR³—;
Y is —CHR⁴—;
R¹ is halo or -L-(C₆₋₁₀ aryl), wherein aryl is optionally substituted with R^{1a};
R³ is C₇₋₁₆ arylalkyl, wherein aryl in arylalkyl is substituted with R^{3a};
R⁴ is H;
R^{3a} is halo or -M-(C₆₋₁₀ aryl), wherein aryl is optionally substituted with R^{3b};
each of R^{1b} and R^{3b} is independently selected from the group consisting of halo and cyano;
L and M are independently selected from the group consisting of a bond, —O—, C₁₋₆ alkylene, and 2- to 6-membered heteroalkylene; and
each heterocyclyl is optionally and independently substituted with one or more amine protecting groups.

In some embodiments, the invention provides a compound selected from Compounds 1, 3, 5, 6, 7, 9, 11, 13, 14, 15, 16, 17, 18, 19, 20, 21, 22, 23, 24, 25, 28, 29, 31, 32, 33, 34, 35, 36, 37, 38, 39, 40, 41, 42, 43, 44, 45, 46, 47, 48, 49, 50, and 51 as shown in Table 1 below, or a pharmaceutical salt thereof.

In some embodiments, the invention provides a compound selected from Compounds 1, 3, 5, 6, 7, 9, 11, 13, 14, 15, 16, 17, 18, 19, 20, 21, 22, 23, 24, 25, 28, 29, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, and 79 as shown in Table 1 below, or a pharmaceutical salt thereof.

TABLE 1
Glycolate Oxidase Inhibitors.

| Compound No. | Chemical Structure |
|---|---|
| 1 | 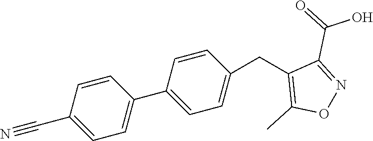 [SMILES: Cc1onc(C(=O)O)c1Cc1ccc(-c2ccc(C#N)cc2)cc1] |
| 2 | 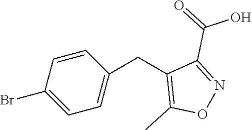 [SMILES: Cc1onc(C(=O)O)c1Cc1ccc(Br)cc1] |
| 3 | 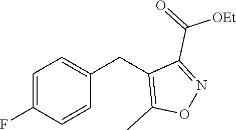 [SMILES: CCOC(=O)c1noc(C)c1Cc1ccc(F)cc1] |
| 4 | 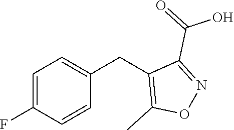 [SMILES: Cc1onc(C(=O)O)c1Cc1ccc(F)cc1] |
| 5 | 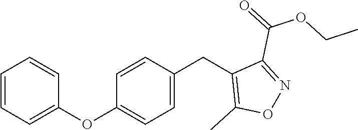 [SMILES: CCOC(=O)c1noc(C)c1Cc1ccc(Oc2ccccc2)cc1] |
| 6 | 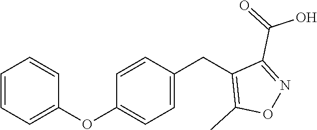 [SMILES: Cc1onc(C(=O)O)c1Cc1ccc(Oc2ccccc2)cc1] |
| 7 | 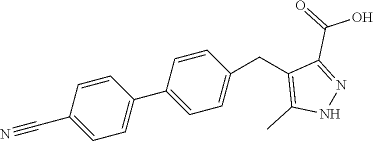 [SMILES: Cc1[nH]nc(C(=O)O)c1Cc1ccc(-c2ccc(C#N)cc2)cc1] |
| 8 | 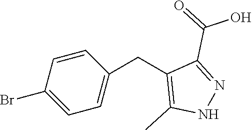 [SMILES: Cc1[nH]nc(C(=O)O)c1Cc1ccc(Br)cc1] |
| 9 | 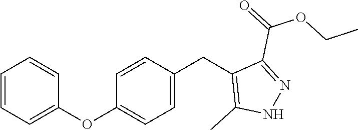 [SMILES: CCOC(=O)c1n[nH]c(C)c1Cc1ccc(Oc2ccccc2)cc1] |
| 10 | 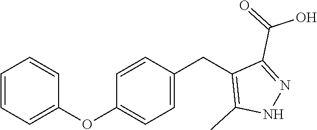 [SMILES: Cc1[nH]nc(C(=O)O)c1Cc1ccc(Oc2ccccc2)cc1] |
| 11 | 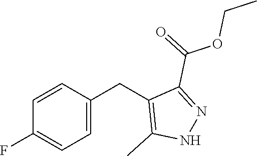 [SMILES: CCOC(=O)c1n[nH]c(C)c1Cc1ccc(F)cc1] |
| 12 | 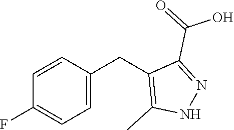 [SMILES: Cc1[nH]nc(C(=O)O)c1Cc1ccc(F)cc1] |
| 13 | 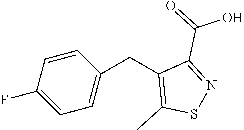 [SMILES: Cc1snc(C(=O)O)c1Cc1ccc(F)cc1] |
| 14 | 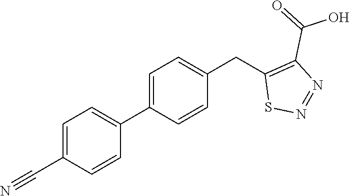 [SMILES: N#Cc1ccc(-c2ccc(Cc3snnc3C(=O)O)cc2)cc1] |
| 15 | 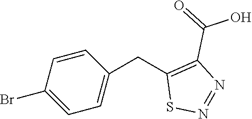 [SMILES: O=C(O)c1nnsc1Cc1ccc(Br)cc1] |
| 16 | 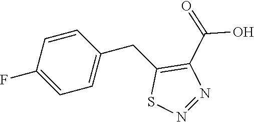 [SMILES: O=C(O)c1nnsc1Cc1ccc(F)cc1] |
| 17 | 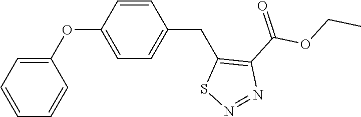 [SMILES: CCOC(=O)c1nnsc1Cc1ccc(Oc2ccccc2)cc1] |
| 18 | 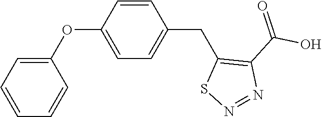 [SMILES: O=C(O)c1nnsc1Cc1ccc(Oc2ccccc2)cc1] |
| 19 | 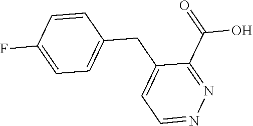 [SMILES: O=C(O)c1nnccc1Cc1ccc(F)cc1] |
| 20 | 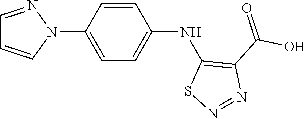 [SMILES: O=C(O)c1nnsc1Nc1ccc(-n2cccn2)cc1] |
| 21 | 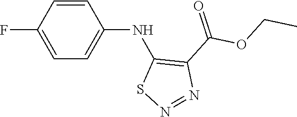 [SMILES: CCOC(=O)c1nnsc1Nc1ccc(F)cc1] |
| 22 | 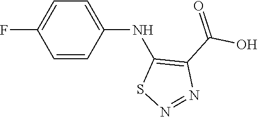 [SMILES: O=C(O)c1nnsc1Nc1ccc(F)cc1] |
| 23 | 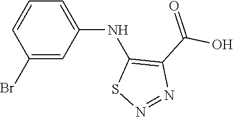 [SMILES: O=C(O)c1nnsc1Nc1cccc(Br)c1] |
| 24 | 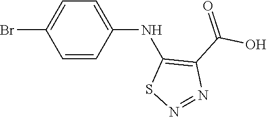 [SMILES: O=C(O)c1nnsc1Nc1ccc(Br)cc1] |
| 25 | 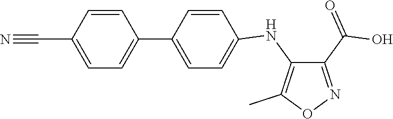 [SMILES: Cc1onc(C(=O)O)c1Nc1ccc(-c2ccc(C#N)cc2)cc1] |
| 26 | 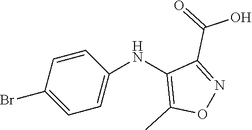 [SMILES: Cc1onc(C(=O)O)c1Nc1ccc(Br)cc1] |
| 27 | 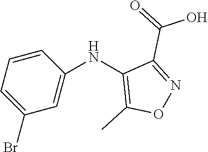 [SMILES: Cc1onc(C(=O)O)c1Nc1cccc(Br)c1] |
| 28 | 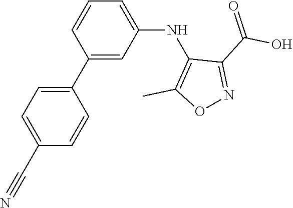 [SMILES: Cc1onc(C(=O)O)c1Nc1cccc(-c2ccc(C#N)cc2)c1] |
| 29 | 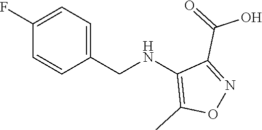 [SMILES: Cc1onc(C(=O)O)c1NCc1ccc(F)cc1] |
| 30 | 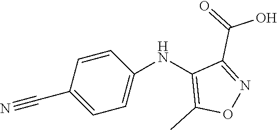 [SMILES: Cc1onc(C(=O)O)c1Nc1ccc(C#N)cc1] |
| 31 | 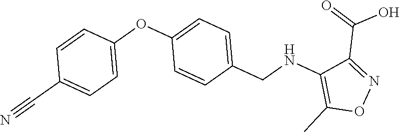 [SMILES: Cc1onc(C(=O)O)c1NCc1ccc(Oc2ccc(C#N)cc2)cc1] |
| 32 | 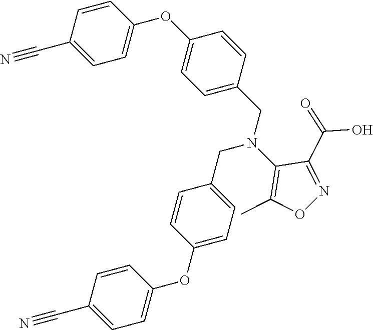 [SMILES: Cc1onc(C(=O)O)c1N(Cc1ccc(Oc2ccc(C#N)cc2)cc1)Cc1ccc(Oc2ccc(C#N)cc2)cc1] |
| 33 | 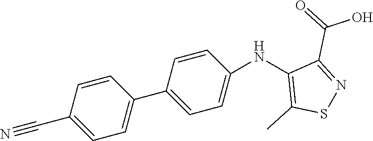 [SMILES: Cc1snc(C(=O)O)c1Nc1ccc(-c2ccc(C#N)cc2)cc1] |
| 34 | 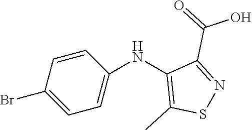 [SMILES: Cc1snc(C(=O)O)c1Nc1ccc(Br)cc1] |
| 35 | 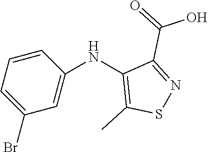 [SMILES: Cc1snc(C(=O)O)c1Nc1cccc(Br)c1] |
| 36 | 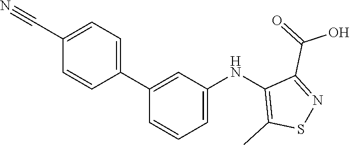 [SMILES: Cc1snc(C(=O)O)c1Nc1cccc(-c2ccc(C#N)cc2)c1] |
| 37 | 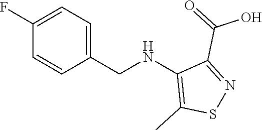 [SMILES: Cc1snc(C(=O)O)c1NCc1ccc(F)cc1] |
| 38 | 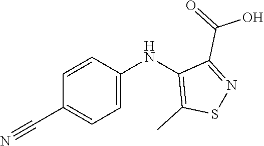 [SMILES: Cc1snc(C(=O)O)c1Nc1ccc(C#N)cc1] |
| 39 | 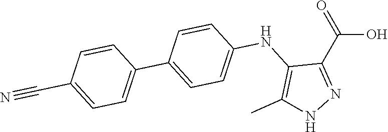 [SMILES: Cc1[nH]nc(C(=O)O)c1Nc1ccc(-c2ccc(C#N)cc2)cc1] |
| 40 | 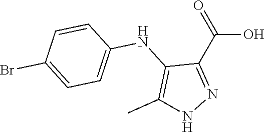 [SMILES: Cc1[nH]nc(C(=O)O)c1Nc1ccc(Br)cc1] |
| 41 | 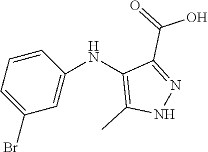 [SMILES: Cc1[nH]nc(C(=O)O)c1Nc1cccc(Br)c1] |
| 42 | 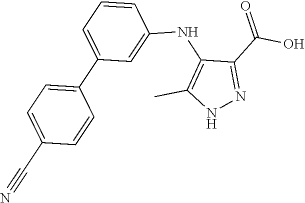 [SMILES: Cc1[nH]nc(C(=O)O)c1Nc1cccc(-c2ccc(C#N)cc2)c1] |
| 43 | 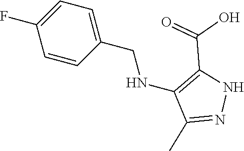 [SMILES: Cc1n[nH]c(C(=O)O)c1NCc1ccc(F)cc1] |
| 44 | 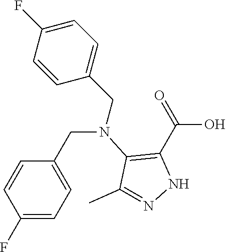 [SMILES: Cc1n[nH]c(C(=O)O)c1N(Cc1ccc(F)cc1)Cc1ccc(F)cc1] |
| 45 | 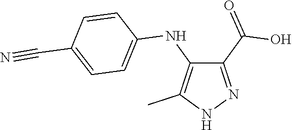 [SMILES: Cc1[nH]nc(C(=O)O)c1Nc1ccc(C#N)cc1] |
| 46 | 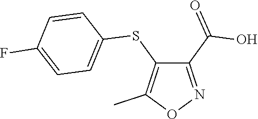 [SMILES: Cc1onc(C(=O)O)c1Sc1ccc(F)cc1] |
| 47 | 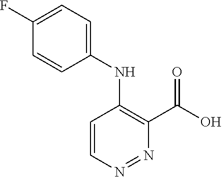 [SMILES: O=C(O)c1nnccc1Nc1ccc(F)cc1] |
| 48 | 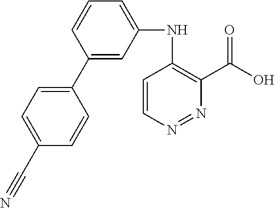 [SMILES: N#Cc1ccc(-c2cccc(Nc3ccnnc3C(=O)O)c2)cc1] |
| 49 | 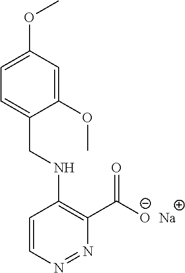 [SMILES: COc1ccc(CNc2ccnnc2C(=O)[O-])c(OC)c1 ; [Na+]] |
| 50 | 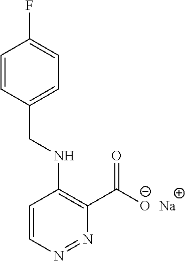 [SMILES: O=C([O-])c1nnccc1NCc1ccc(F)cc1 ; [Na+]] |
| 51 | 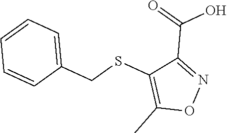 [SMILES: Cc1onc(C(=O)O)c1SCc1ccccc1] |
| 52 | 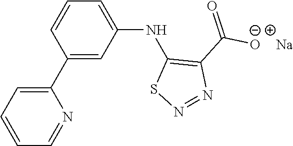 [SMILES: O=C([O-])c1nnsc1Nc1cccc(-c2ccccn2)c1 ; [Na+]] |
| 53 | 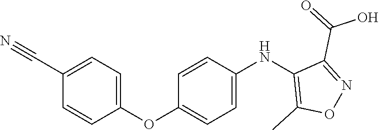 [SMILES: Cc1onc(C(=O)O)c1Nc1ccc(Oc2ccc(C#N)cc2)cc1] |
| 54 | 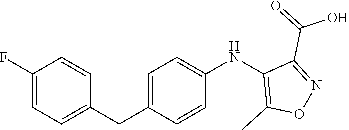 [SMILES: Cc1onc(C(=O)O)c1Nc1ccc(Cc2ccc(F)cc2)cc1] |
| 55 | 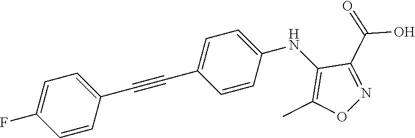 [SMILES: Cc1onc(C(=O)O)c1Nc1ccc(C#Cc2ccc(F)cc2)cc1] |
| 56 | 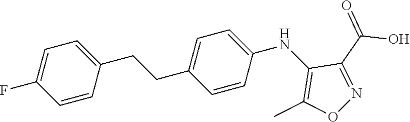 [SMILES: Cc1onc(C(=O)O)c1Nc1ccc(CCc2ccc(F)cc2)cc1] |
| 57 | 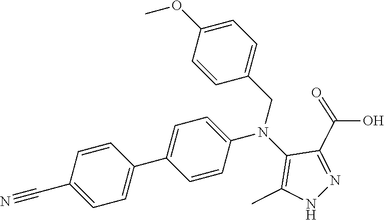 [SMILES: COc1ccc(CN(c2ccc(-c3ccc(C#N)cc3)cc2)c2c(C(=O)O)n[nH]c2C)cc1] |
| 58 | 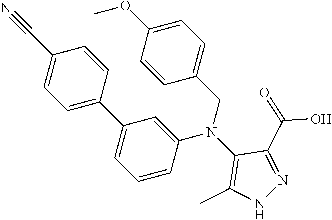 [SMILES: COc1ccc(CN(c2cccc(-c3ccc(C#N)cc3)c2)c2c(C(=O)O)n[nH]c2C)cc1] |
| 59 | 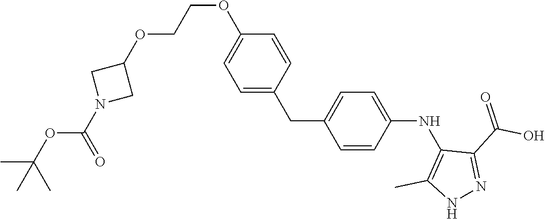 [SMILES: Cc1[nH]nc(C(=O)O)c1Nc1ccc(Cc2ccc(OCCOC3CN(C(=O)OC(C)(C)C)C3)cc2)cc1] |
| 60 | 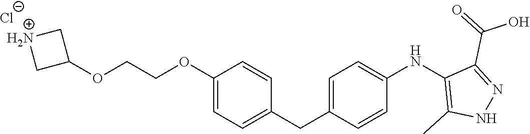 [SMILES: [Cl-] ; Cc1[nH]nc(C(=O)O)c1Nc1ccc(Cc2ccc(OCCOC3C[NH2+]C3)cc2)cc1] |
| 61 | 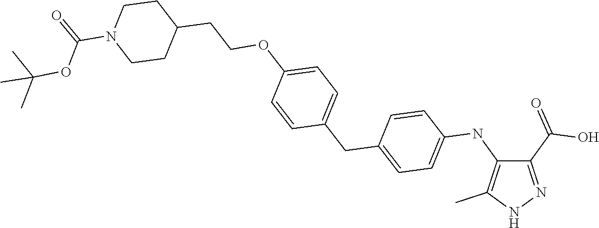 [SMILES: Cc1[nH]nc(C(=O)O)c1Nc1ccc(Cc2ccc(OCCC3CCN(C(=O)OC(C)(C)C)CC3)cc2)cc1] |
| 62 | 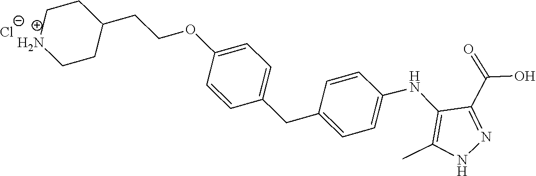 [SMILES: [Cl-] ; Cc1[nH]nc(C(=O)O)c1Nc1ccc(Cc2ccc(OCCC3CC[NH2+]CC3)cc2)cc1] |
| 63 | 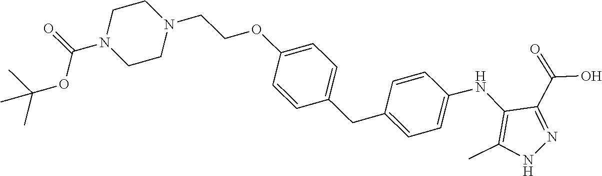 [SMILES: Cc1[nH]nc(C(=O)O)c1Nc1ccc(Cc2ccc(OCCN3CCN(C(=O)OC(C)(C)C)CC3)cc2)cc1] |
| 64 | 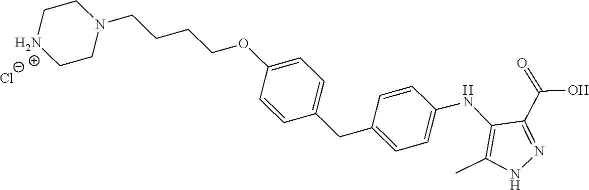 [SMILES: [Cl-] ; Cc1[nH]nc(C(=O)O)c1Nc1ccc(Cc2ccc(OCCCCN3CC[NH2+]CC3)cc2)cc1] |
| 65 | 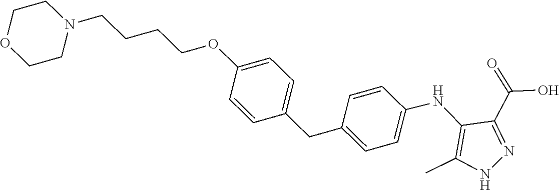 [SMILES: Cc1[nH]nc(C(=O)O)c1Nc1ccc(Cc2ccc(OCCCCN3CCOCC3)cc2)cc1] |
| 66 | 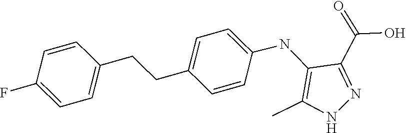 [SMILES: Cc1[nH]nc(C(=O)O)c1Nc1ccc(CCc2ccc(F)cc2)cc1] |
| 67 | 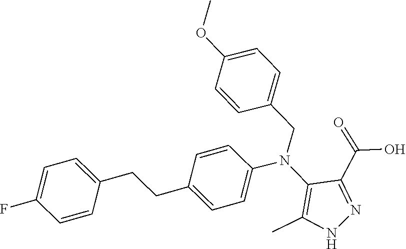 [SMILES: COc1ccc(CN(c2ccc(CCc3ccc(F)cc3)cc2)c2c(C(=O)O)n[nH]c2C)cc1] |
| 68 | 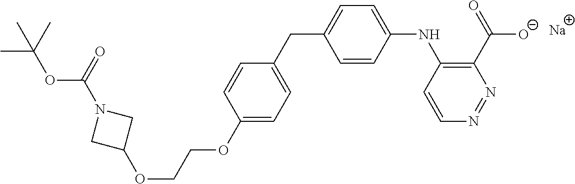 [SMILES: CC(C)(C)OC(=O)N1CC(OCCOc2ccc(Cc3ccc(Nc4ccnnc4C(=O)[O-])cc3)cc2)C1 ; [Na+]] |
| 69 | 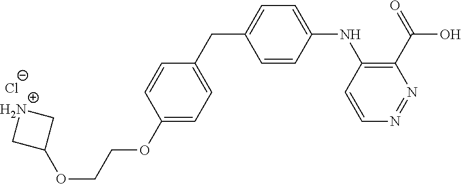 [SMILES: [Cl-] ; O=C(O)c1nnccc1Nc1ccc(Cc2ccc(OCCOC3C[NH2+]C3)cc2)cc1] |
| 70 | 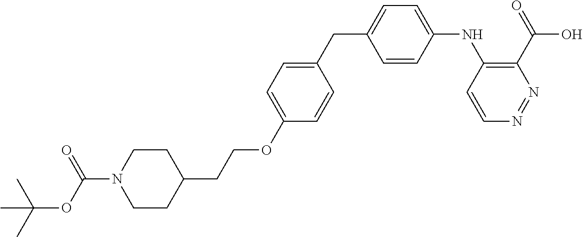 [SMILES: CC(C)(C)OC(=O)N1CCC(CCOc2ccc(Cc3ccc(Nc4ccnnc4C(=O)O)cc3)cc2)CC1] |
| 71 | 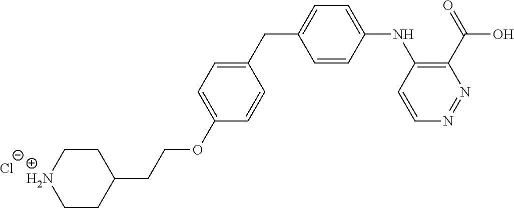 [SMILES: [Cl-] ; O=C(O)c1nnccc1Nc1ccc(Cc2ccc(OCCC3CC[NH2+]CC3)cc2)cc1] |
| 72 | 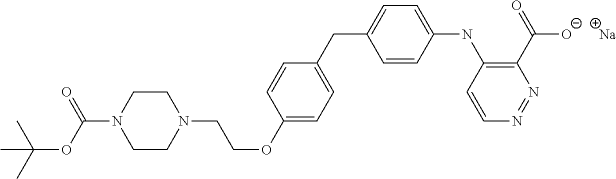 [SMILES: CC(C)(C)OC(=O)N1CCN(CCOc2ccc(Cc3ccc(Nc4ccnnc4C(=O)[O-])cc3)cc2)CC1 ; [Na+]] |
| 73 | 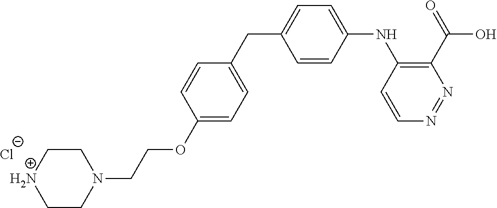 [SMILES: [Cl-] ; O=C(O)c1nnccc1Nc1ccc(Cc2ccc(OCCN3CC[NH2+]CC3)cc2)cc1] |
| 74 | 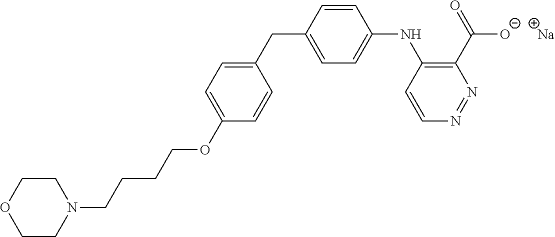 [SMILES: O=C([O-])c1nnccc1Nc1ccc(Cc2ccc(OCCCCN3CCOCC3)cc2)cc1 ; [Na+]] |
| 75 | 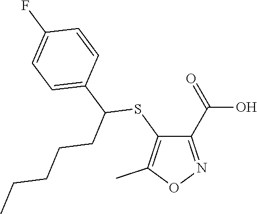 [SMILES: CCCCCC(Sc1c(C(=O)O)noc1C)c1ccc(F)cc1] |
| 76 | 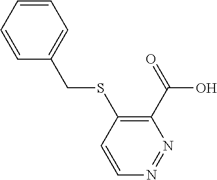 [SMILES: O=C(O)c1nnccc1SCc1ccccc1] |
| 77 | 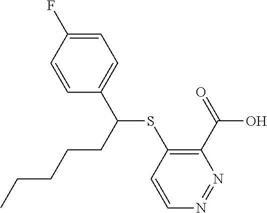 [SMILES: CCCCCC(Sc1ccnnc1C(=O)O)c1ccc(F)cc1] |
| 78 | 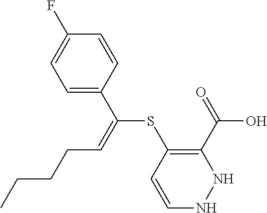 [SMILES: CCCC/C=C(/SC1=C(C(=O)O)NNC=C1)c1ccc(F)cc1] |
| 79 | 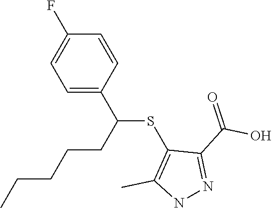 [SMILES: CCCCCC(Sc1c(C(=O)O)n[nH]c1C)c1ccc(F)cc1] |

In some embodiments, the invention provides a compounds selected from the group consisting of:

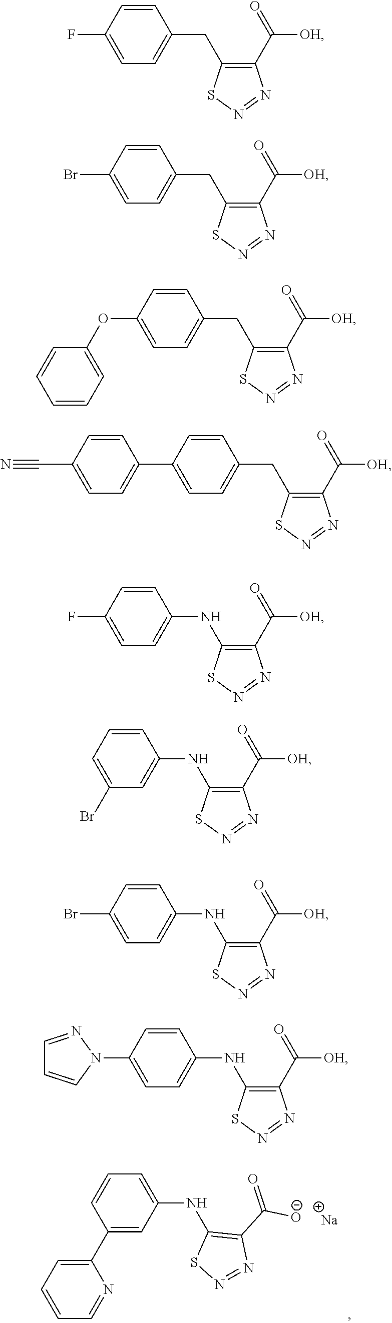

and pharmaceutically acceptable salts thereof.

In some embodiments, the invention provides a compound selected from the group consisting of:

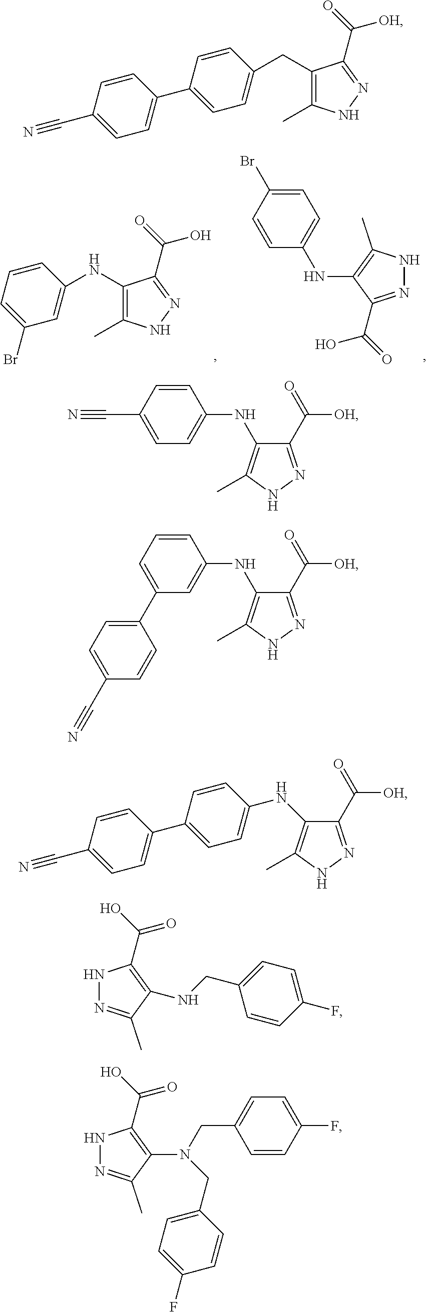

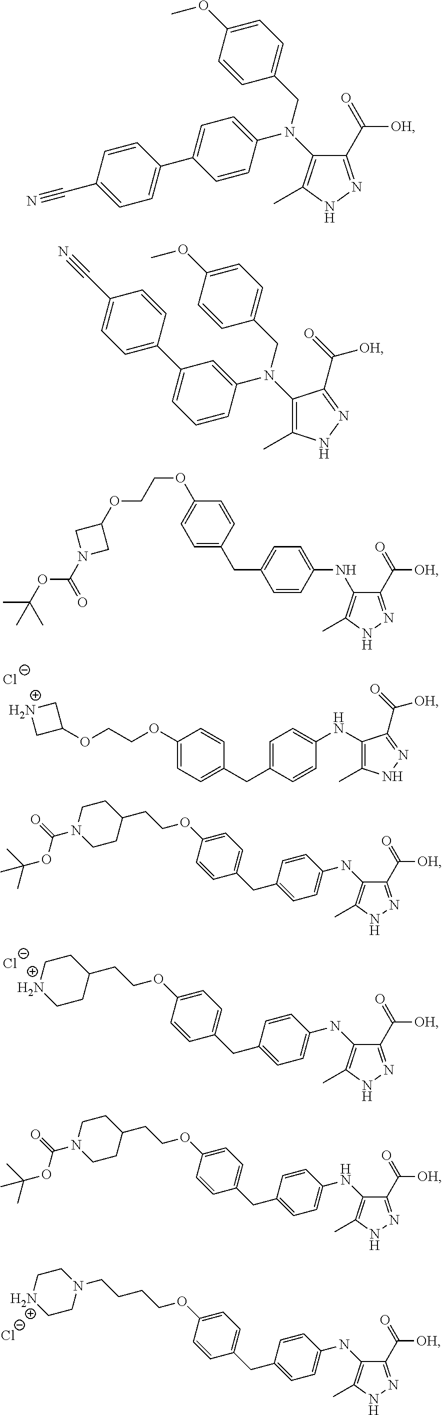

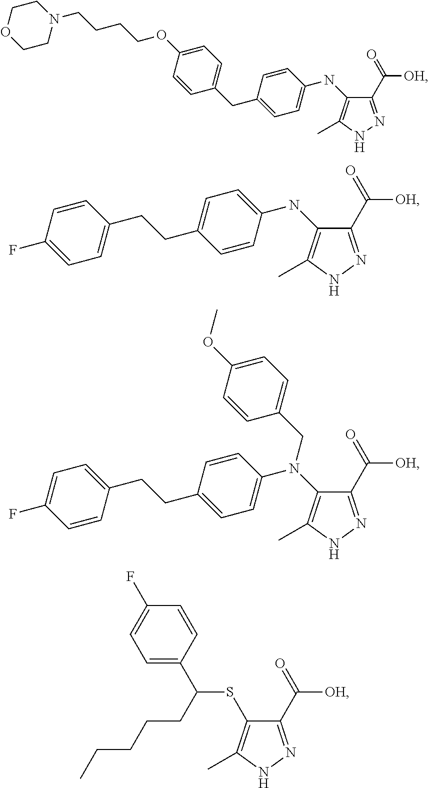

and pharmaceutically acceptable salts thereof.

In some embodiments, the invention provides a compound selected from the group consisting of:

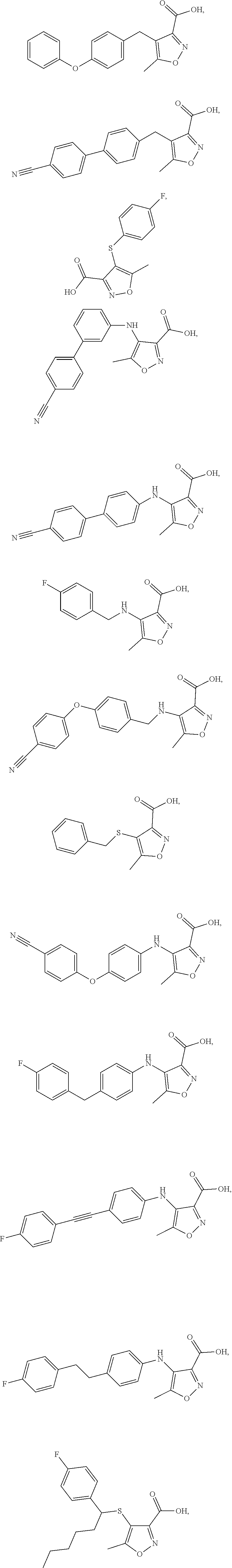

and pharmaceutically acceptable salts thereof.

In some embodiments, the invention provides a compound selected from the group consisting of:

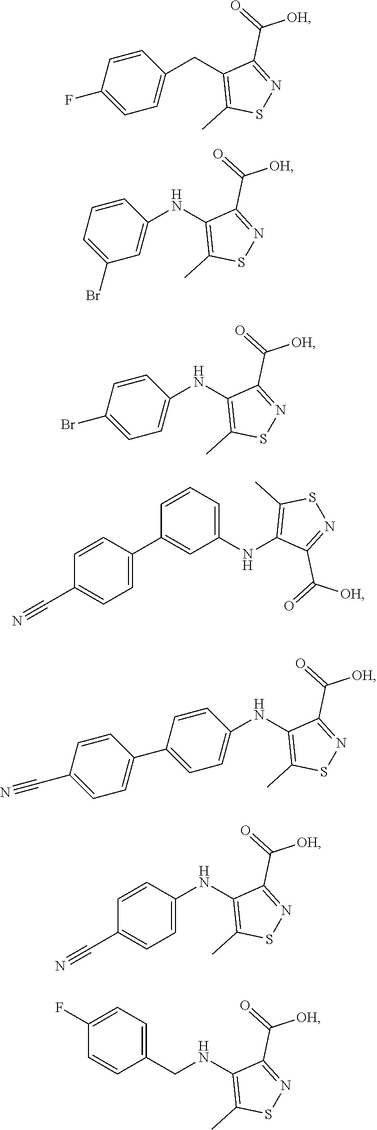

and pharmaceutically acceptable salts thereof.

In some embodiments, the invention provides a compound selected from the group consisting of:

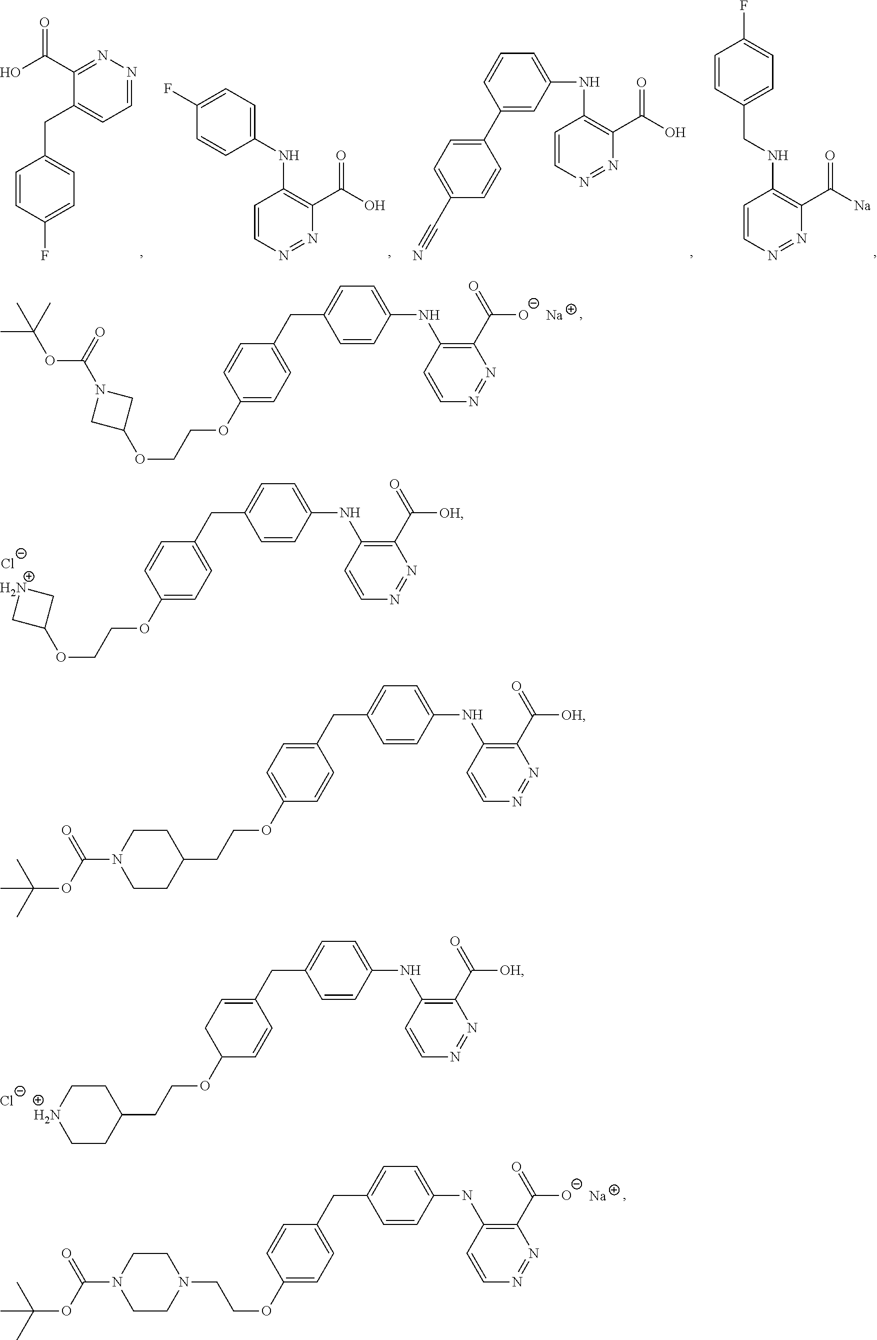

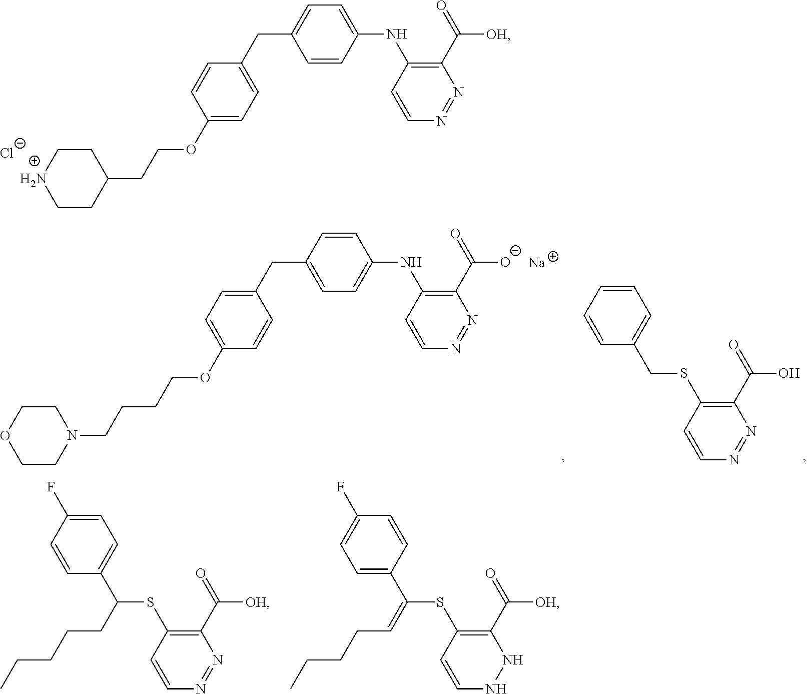

and pharmaceutically acceptable salts thereof.

In some embodiments, the compounds of the invention do not include the following:

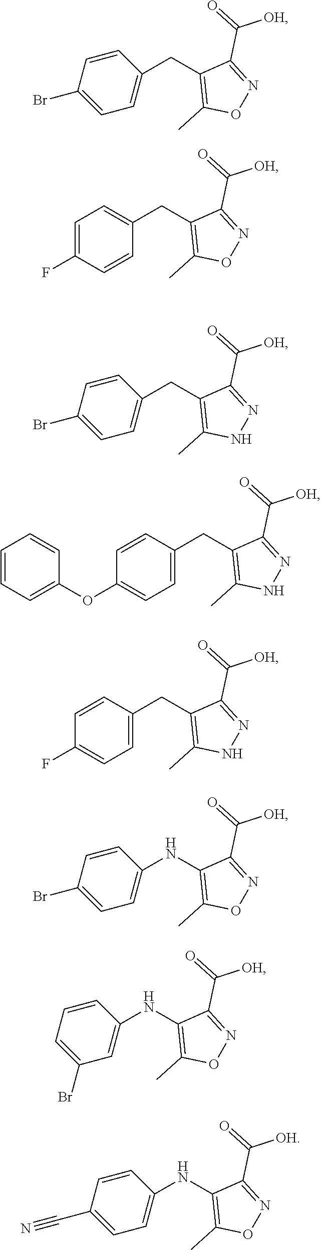

### IV. Pharmaceutical Compositions

The invention also provides pharmaceutical compositions for the administration of GO inhibitors. Accordingly, one embodiment of the invention provides a pharmaceutical composition containing a compound as described above and one or more pharmaceutically acceptable excipients. The pharmaceutical compositions can be prepared by any of the methods well known in the art of pharmacy and drug delivery. In general, methods of preparing the compositions include the step of bringing the active ingredient into association with a carrier containing one or more accessory ingredients. The pharmaceutical compositions are typically prepared by uniformly and intimately bringing the active ingredient into association with a liquid carrier or a finely divided solid carrier or both, and then, if necessary, shaping the product into the desired formulation. The compositions can be conveniently prepared and/or packaged in unit dosage form.

The pharmaceutical compositions can be in the form of a sterile injectable aqueous or oleaginous solutions and suspensions. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Aqueous suspensions contain the active ingredient in admixture with excipients suitable for the manufacture of aqueous suspensions. Such excipients include, but are not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate.

Oily suspensions can be formulated by suspending the active ingredient in a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol. These compositions can be preserved by the addition of an anti-oxidant such as ascorbic acid.

Dispersible powders and granules (suitable for preparation of an aqueous suspension by the addition of water) can contain the active ingredient in admixture with a dispersing agent, wetting agent, suspending agent, or combinations thereof. Additional excipients can also be present.

The pharmaceutical compositions of the invention can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

Pharmaceutical compositions containing GO inhibitors can also be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredient is mixed with water or an oil medium (such as peanut oil, liquid paraffin, or olive oil).

Transdermal delivery of GO inhibitors can be accomplished by means of iontophoretic patches and the like. The compound can also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter and polyethylene glycols.

In some embodiments, the pharmaceutical composition includes a GO inhibitor as described herein and one or more additional active agents for treating kidney stones. Examples of such active agents include, but are not limited to, thiazides (e.g., bendroflumethiazide, chlorothiazide, chlorthalidone, hydrochlorothiazide, indapamide, methyclothiazide, metolazone, polythiazide, and the like); citrate salts (e.g., sodium citrate, potassium citrate, and the like); phosphate salts (e.g., monopotassium phosphate, dipotassium phosphate, and the like); vitamin B₆ compounds (e.g., pyridoxine, pyridoxal, pyridoxamine, and the like); cystine-binding thiol compounds (e.g., α-mercaptopropionylglycine, D-penicillamine, captopril, and the like); purine analog xanthine oxidase inhibitors (e.g., allopurinol, oxypurinol, and the like); and other xanthine oxidase inhibitors (e.g., febuxostate, topiroxostat, and the like).

### V. Methods of Treating Kidney Stone Disease and Primary Hyperoxaluria

PH1 has a prevalence of 1-3 per million individuals and an incidence of 1-9: 100,000 live births per year in Europe [Salido, supra]. PH1 is caused by mutations of the gene encoding peroxisomal enzyme AGT, which fails to detoxify glyoxylate and leads to a marked increase in oxalate synthesis by the liver. In PH1, excreted urinary oxalate (UOx) is elevated leading to the production of insoluble calcium oxalate (CaOx) crystals which tend to precipitate primarily in the kidney, forming kidney stones and diffuse nephrocalcinosis [Kaufman, supra]. This impairs renal function which progresses to end-stage renal disease (ESRD). Once renal function declines to a glomerular filtration rate (GFR) below 30 mL/min/1.73 m², the amount of oxalate produced by the liver can no longer be cleared by the kidneys, leading to systemic deposition of CaOx (oxalosis). First symptoms of PH1 include hematuria, abdominal pain, passage of a stone, or repeated urinary tract infections. The initial diagnosis is based on clinical and sonographic findings, and UOx assessment. AGT activity assessment in a liver biopsy and/or DNA analysis is required to confirm a PH1 diagnosis and to initiate conservative treatment (high fluid intake, pyridoxine, CaOx crystallization inhibitors), aimed at maintaining renal function. The most effective treatment for PH1 is liver transplantation (LTx), alone (pre-emptive) or combined with kidney transplantation [Cochat, et al. (2012) Nephrol Dial Transplant. 27: 1729].

Secondary hyperoxaluria (SH) may occur because of excess dietary intake of oxalate precursors or oxalate-rich foods or may be the result of well-known oxalate hyperabsorptive conditions, such as inflammatory bowel diseases, post large bowel resection or small intestine bypass operations. However, most cases of secondary hyperoxaluria are idiopathic [Bhasin, et al. (2015) World J Nephrol. 4: 235]. Clinicians routinely recommend a low oxalate diet to patients with idiopathic urolithiasis. However, the effect of dietary oxalate intake on urinary oxalate levels is controversial. A large amount of urinary oxalate is derived from the endogenous metabolism of glycine, glycolate, hydroxyproline, and vitamin C, and estimates of the proportion of urinary oxalate derived from dietary oxalate vary widely from 10% to 50% [Holmes, supra].

In another embodiment, the invention provides a method for treating primary hyperoxaluria, type I (PHI). The method includes administering to a subject in need thereof a therapeutically effective amount of a compound according to Formula I:

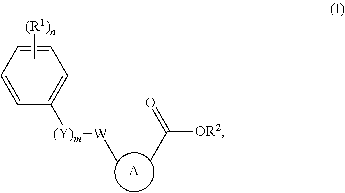

or a pharmaceutically acceptable salt thereof, wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₁₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₁₋₆ alkenylene, C₁₋₆ alkynylene, and 2- to 6-membered heteroalkylene; and
each heterocyclyl is optionally and independently substituted with one or more amine protecting groups;
provided that if ring A is 1,2,3-thiadiazol-4,5-diyl, W is S, subscript n is 1, and subscript m is 0, then R¹ is other than 4-chloro;
   thereby treating the PH1.

In another embodiment, the invention provides a method for treating kidney stones. The method includes administering to a subject in need thereof a therapeutically effective amount of a compound according to Formula I:

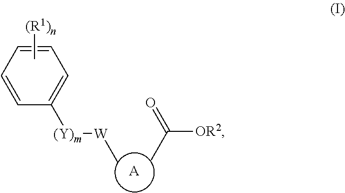

or a pharmaceutically acceptable salt thereof, wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —CHR⁴—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a};
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L and M are independently selected from the group consisting of a bond, —O—, C₁₋₆ alkylene, and 2- to 6-membered heteroalkylene; and
each heterocyclyl is optionally and independently substituted with one or more amine protecting groups;
provided that if ring A is 1,2,3-thiadiazol-4,5-diyl, W is S, subscript n is 1, and subscript m is 0, then R¹ is other than 4-chloro;
   thereby treating the kidney stones.

GO inhibitors can be administered at any suitable dose in the methods of the invention. In general, a GO inhibitor will be administered at a dose ranging from about 0.1 milligrams to about 1000 milligrams per kilogram of a subject's body weight (i.e., about 0.1-1000 mg/kg). The dose of the GO inhibitor can be, for example, about 0.1-1000 mg/kg, or about 1-500 mg/kg, or about 25-250 mg/kg, or about 50-125 mg/kg. The dose of the GO inhibitor can be about 0.1-1 mg/kg, or about 1-50 mg/kg, or about 50-100 mg/kg, or about 100-150 mg/kg, or about 150-200 mg/kg, or about 200-250 mg/kg, or about 250-300 mg/kg, or about 350-400 mg/kg, or about 450-500 mg/kg, or about 500-550 mg/kg, or about 550-600 mg/kg, or about 600-650 mg/kg, or about 650-700 mg/kg, or about 700-750 mg/kg, or about 750-800 mg/kg, or about 800-850 mg/kg, or about 850-900 mg/kg, or about 900-950 mg/kg, or about 950-1000 mg/kg. The dose of the GO inhibitor can be about 1, 2, 3, 4, 5, 10, 15, 20, 25, 30, 35, 40, 45, 50, 55, 60, 65, 70, 75, 85, 90, 95, 100, 150, 200, 250, 300, 350, 400, 450, 500, 550, 600, 650, 700, 750, 800, 850, 900, 950 or 1000 mg/kg. The GO inhibitor can be administered, orally, topically, parenterally, intravenously, intraperitoneally, intramuscularly, intralesionally, intranasally, subcutaneously, or intrathecally using a suitable vehicle, including any of the compositions described above. Alternatively, the GO inhibitor can be administered via a suppository or via implantation of a slow-release device, e.g., a mini-osmotic pump.

The dosages can be varied depending upon the requirements of the patient, the severity of the kidney stones and/or PH1 being treated, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the kidney stones and/or PH1.

Administration of the GO inhibitor can be conducted for a period of time which will vary depending upon the nature of the particular disorder, its severity and the overall condition of the patient. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a patient can be monitored for changes in his or her condition and for alleviation of the symptoms of the disorder. The dosage of the GO-inhibitor can either be increased in the event the patient does not respond significantly to a particular dosage level, or the dose can be decreased if an alleviation of the symptoms is observed, or if unacceptable side effects are seen with a particular dosage. The dosage regimen can consist of two or more different interval sets. For example, a first part of the dosage regimen can be administered to a subject multiple times daily, daily, every other day, or every third day. The dosing regimen can start with dosing the subject every other day, every third day, weekly, biweekly, or monthly. The first part of the dosing regimen can be conducted, for example, for up to 30 days, such as 7, 14, 21, or 30 days. A subsequent second part of the dosing regimen with a different interval administration administered weekly, every 14 days, or monthly can optionally follow, continuing for 4 weeks up to two years or longer, such as 4, 6, 8, 12, 16, 26, 32, 40, 52, 63, 68, 78, or 104 weeks. Alternatively, if the symptoms go into remission or generally improves, the dosage may be maintained or kept at lower than maximum amount. If kidney stones reappear or PH1 symptoms worsen, the first dosage regimen can be resumed until an improvement is seen, and the second dosing regimen can be implemented again. This cycle can be repeated multiple times as necessary.

### VI. Examples

### Example 1. 4-((4′-Cyano-[1, 1′-biphenyl]-4-yl)methyl)-5-methylisoxazole-3-carboxylic Acid (1)

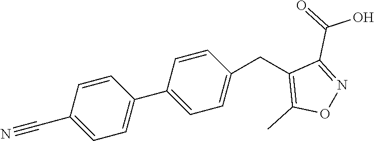 [SMILES: Cc1onc(C(=O)O)c1Cc1ccc(-c2ccc(C#N)cc2)cc1]

The title compound was prepared according to Scheme 1. To a stirred solution of ethyl 2, 4-dioxopentanoate (5.0 g, 31.606 mmol) in ethanol:water (1:1, 90 mL) was added O-methylhydroxylamine hydrochloride (1.8 g, 22.124 mmol) at ambient temperature and the reaction mixture was stirred for 16 h. The resulting reaction mixture was poured into water (200 mL) and extracted with EtOAc (2×100 mL). The combined organic phase was dried over Na₂SO₄, filtered and concentrated under reduced pressure. The resulting crude material was purified by column chromatography (18% EtOAc in hexane) yielding ethyl (Z)-2-(methoxyimino)-4-oxopentanoate (intermediate A; 3.9 g, 20.856 mmol). LCMS: Method B, 1.523 min, MS: ES+ 188.1 (M+1). See Tables 2A-2C for LCMS parameters.

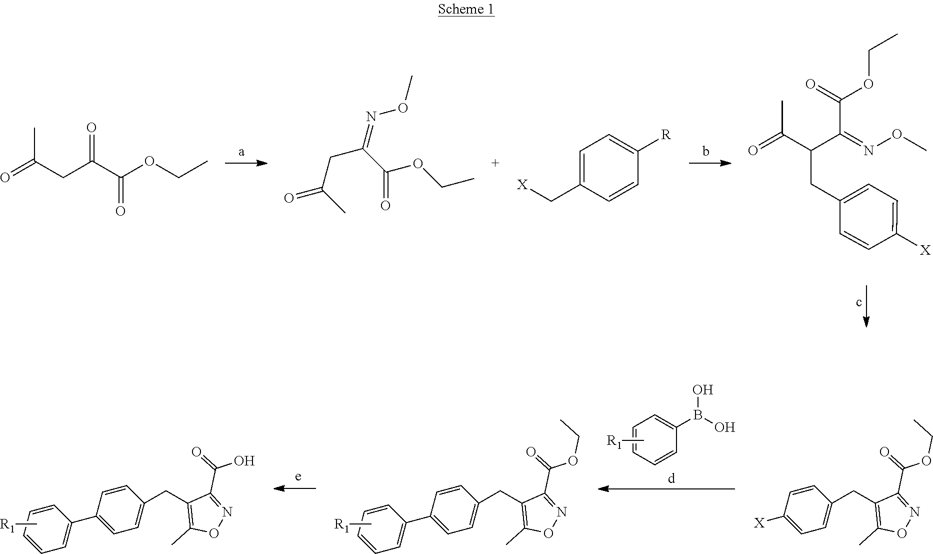

TABLE 2A
LCMS Instrumentation and Experimental Parameters

| | Method A | Method B | Method C | Method D |
|---|---|---|---|---|
| Instrument: | UPLC AQUITY | Waters Alliance | Waters Alliance | Waters Alliance |
| | with PDA detector | 2690 and 996 PDA | 2690 and 996 PDA | 2690 and 996 PDA |
| | and QDA | detector with | detector with | detector with |
| | | Micromass ZQ | Micromass ZQ | Micromass ZQ |
| Column: | C18, 50 * 2.1 mm, | C18, 50 × 4.6 mm, | C18, 50 * 4.6 mm, | C18, 150 * 4.6 mm, |
| | 1.6 μm | 3.5 μm | 5 μm | 3.5 μm |
| Mobile Phase: | (A) 0.1% Formic | (A) 10 mM | (A ) 0.05% | (A ) 0.05% |
| | acid in Milli Q | Ammonium | Ammonium | Ammonium |
| | water (pH = 2.70) | Bicarbonate in | Acetate and 0.1% | Acetate and 0.1% |
| | (B ) Acetonitrile | Milli-Qwater | Formic acid in | Formic acid in |
| | | (pH = 7.35) | Milli-Q water | Milli-Q water |
| | | (B) Methanol | (pH = 4.6) | (pH = 4.6) |
| | | | (B ) Methanol | (B ) Methanol |
| Flow Rate: | 0.800 mL/min | 1.200 mL/min | 1.000 mL/min | 0.800 mL/min |
| Run Time: | 6 min | 7 min | 7 min | 17 min |

TABLE 2B
LCMS Solvent Gradient

| Method A | | | Method B | | | Method C | | | Method D | | |
|---|---|---|---|---|---|---|---|---|---|---|---|
| t | | | t | | | t | | | t | | |
| (min) | % A | % B | (min) | % A | % B | (min) | % A | % B | (min) | % A | % B |
| 0.00 | 90 | 10 | 0.00 | 90 | 10 | 0.00 | 90 | 10 | 0.00 | 90 | 10 |
| 0.75 | 90 | 10 | 1.00 | 90 | 10 | 1.00 | 90 | 10 | 7.00 | 10 | 90 |
| 2.80 | 10 | 90 | 4.00 | 00 | 100 | 4.00 | 00 | 100 | 9.00 | 00 | 100 |
| 4.50 | 00 | 100 | 6.00 | 00 | 100 | 6.00 | 00 | 100 | 1400 | 00 | 100 |
| 4.60 | 00 | 100 | 6.50 | 90 | 10 | 6.50 | 90 | 10 | 14.01 | 90 | 10 |
| 4.70 | 90 | 10 | 7.00 | 90 | 10 | 7.00 | 90 | 10 | 17.00 | 90 | 10 |
| 6.00 | 90 | 10 | | | | | | | | | |

TABLE 2C
LCMS Mass Spectrometer Parameters

| | Method A | Method B | Method C | Method C |
|---|---|---|---|---|
| Probe | ESI capillary | ESI capillary | ESI capillary | ESI capillary |
| Source Temperature | 120° C. | 100° C. | 100° C. | 100° C. |
| Probe Temperature/ | 600° C.¹ | 200° C.² | 200° C.² | 200° C.² |
| Desolvation temperature | | | | |
| Capillary Voltage | 0.8 KV ( +Ve | 3 KV (+Ve | 3 KV (+Ve | 3 KV (+Ve |
| | and −Ve) | and −Ve) | and −Ve) | and −Ve) |
| Cone Voltage | 10 & 30 V | 10 & 30 V | 10 & 30 V | 10 & 30 V |
| Extractor Voltage | n/a | 2.0 V | 2.0 V | 2.0 V |
| Rf Lens | n/a | 0.1 | 0.1 | 0.1 |
| Desolvation Gas Flow | n/a | 800.0 L/hr | 800.0 L/hr | 800.0 L/hr |
| Extractor Voltage | n/a | 100.0 L/hr | 100.0 L/hr | 100.0 L/hr |
| Mode of Ionization | +Ve and −Ve | +Ve and −Ve | +Ve and −Ve | +Ve and −Ve |

¹Probe temperature
²Desolvation temperature

To a stirred solution of ethyl (Z)-2-(methoxyimino)-4-oxopentanoate (Intermediate A, 2.0 g, 10.684 mmol) in acetonitrile (20 mL) were added K₂CO₃ (4.4 g, 32.051 mmol) and 4-bromo benzyl bromide (CAS No. 589-15-1, 4.0 g, 16.026 mmol) at ambient temperature and the reaction mixture was stirred for 18 h. The resulting reaction mixture was poured into water (100 mL) and extracted with EtOAc (2×50 mL). The combined organic phase was dried over Na₂SO₄, filtered and concentrated under reduced pressure. The resulting crude material was purified by flash chromatography (7.2% EtOAc in hexane) yielding ethyl (Z)-3-(4-bromobenzyl)-2-(methoxyimino)-4-oxopentanoate (compound 1.3; 1.6 g, 4.506 mmol). LCMS: Method B, 4.712 min, MS: ES+ 373.3 (M+18).

To a stirred solution of ethyl (Z)-3-(4-bromobenzyl)-2-(methoxyimino)-4-oxopentanoate (compound 1.3; 0.8 g, 2.253 mmol) in acetic acid (10 mL) was added hydroxylamine HCl (0.3 g, 2.703 mmol) at ambient temperature. The reaction mixture was heated at 80° C. for 18 h. The resulting reaction mixture was cooled to ambient temperature and poured into saturated NaHCO₃ solution (100 mL). The resulting reaction mixture was diluted with water (50 mL) and extracted with EtOAc (2×50 mL). The combined organic phase was dried over Na₂SO₄, filtered and concentrated under reduced pressure. The resulting crude material was purified by flash chromatography (8% EtOAc in hexane) yielding ethyl 4-(4-bromobenzyl)-5-methylisoxazole-3-carboxylate (compound 1.2; 0.3 g, 0.929 mmol). LCMS: Method B, 4.871 min, MS: ES+ 324.3 (M+1).

To a stirred solution of ethyl 4-(4-bromobenzyl)-5-methylisoxazole-3-carboxylate (compound 1.2; 0.25 g, 0.619 mmol) in 1, 4-dioxane:water (2:1, 12 mL) were added Na₂CO₃ (0.2 g, 1.857 mmol) and 4-cyano phenyl boronic acid (CAS No. 126747-14-6, 0.14 g, 0.928 mmol) at ambient temperature. The reaction mixture was purged with N₂ gas at ambient temperature for 15 minutes. PdCl₂(dppf) (0.04 g, 0.061 mmol) was added to the reaction mixture at ambient temperature and heating was applied. The reaction mixture was stirred at 80° C. for 10 minutes. The resulting reaction mixture was cooled to ambient temperature and poured into water (50 mL). The resulting reaction mixture was extracted with EtOAc (2×20 mL). The combined organic phase was dried over Na₂SO₄, filtered and concentrated under reduced pressure. The resulting crude material was purified by flash chromatography (10.8% EtOAc in hexane) yielding ethyl 4-((4′-cyano-[1,1′-biphenyl]-4-yl)methyl)-5-methylisoxazole-3-carboxylate (compound 1.1; 0.12 g, 0.347 mmol). LCMS: Method B, 4.774 min, MS: ES+ 347.4 (M+1); ¹H NMR (400 MHz, DMSO-d₆) δ ppm: 7.91 (d, J=8.4 Hz, 2H), 7.86 (d, J=8.4 Hz, 2H), 7.69 (d, J=8.4 Hz, 2H), 7.34 (d, J=8.0 Hz, 2H), 4.38 (q, J=6.8, 14.0 Hz 2H), 4.15 (s, 2H), 2.17 (s, 3H), 1.31 (t, J=6.8 Hz 3H).

To a stirred solution of ethyl 4-((4′-cyano-[1,1′-biphenyl]-4-yl)methyl)-5-methylisoxazole-3-carboxylate (compound 1.1; 0.11 g, 0.318 mmol) in THF:water (2:1, 12 mL) was added LiOH.H₂O (0.02 g, 0.349 mmol) at ambient temperature and the reaction mixture was stirred for 18 h. The resulting reaction mixture was poured into saturated NaHCO₃ solution (20 mL) and washed with EtOAc (2×10 mL). The obtained aqueous layer was acidified (pH=4) with dilute HCl and extracted with EtOAc (2×10 mL). The combined organic phase was dried over Na₂SO₄, filtered and concentrated under reduced pressure. The resulting solid was triturated with n-pentane (2×3 mL) and dried yielding 4-((4′-cyano-[1,1′-biphenyl]-4-yl)methyl)-5-methylisoxazole-3-carboxylic acid (compound 1; 0.02 g, 0.063 mmol). LCMS: Method B, 4.023 min, MS: ES+ 319.4 (M+1); ¹H NMR (400 MHz, DMSO-d₆) δ ppm: 14.34 (br s, 1H), 7.91 (d, J=2.0 Hz, 2H), 7.86 (dd, J=2.0, 6.8 Hz, 2H), 7.69 (d, J=8.0 Hz, 2H), 7.33 (d, J=8.4 Hz, 2H), 4.14 (s, 2H), 2.15 (s, 3H).

### Example 2. 4-(4-Bromobenzyl)-5-methylisoxazole-3-carboxylic Acid (2)

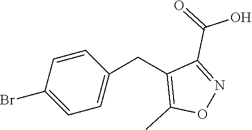 [SMILES: Cc1onc(C(=O)O)c1Cc1ccc(Br)cc1]

The title compound was synthesized by hydrolysis of compound 1.2 following a similar synthetic procedure as described for compound 1 above. LCMS: Method B, 4.015 min, MS: ES+ 296.3 (M+1); ¹H NMR (400 MHz, DMSO-d₆) δ ppm: 14.36 (br s, 1H), 7.48 (d, J=8.4 Hz, 2H), 7.16 (d, J=8.4 Hz, 2H), 4.53 (s, 2H), 2.11 (s, 3H).

### Example 3. Ethyl 4-(4-fluorobenzyl)-5-methylisoxazole-3-carboxylate (3)

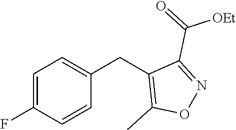 [SMILES: CCOC(=O)c1noc(C)c1Cc1ccc(F)cc1]

The title compound was synthesized via steps a, b, and c of Scheme 1, following similar synthetic procedures as described for preparation of compound 1, using 4-fluoro benzyl bromide (CAS Number 459-46-1) in step b instead of 4-bromo benzyl bromide. LCMS: Method B, 4.650 min, MS: ES+ 264.5 (M+1); ¹H NMR (400 MHz, DMSO-d₆) δ ppm: 7.27-7.23 (m, 2H), 7.15-7.10 (m, 2H), 4.37 (q, J=8.0, 16.0 Hz, 2H), 4.07 (s, 2H), 2.14 (s, 3H), 1.30 (t, J=8.0 Hz, 3H).

### Example 4. 4-(4-Fluorobenzyl)-5-methylisoxazole-3-carboxylic Acid (4)

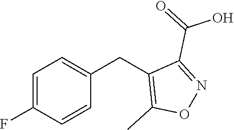 [SMILES: Cc1onc(C(=O)O)c1Cc1ccc(F)cc1]

The title compound was synthesized by hydrolysis of compound 3, ethyl 4-(4-fluorobenzyl)-5-methylisoxazole-3-carboxylate, following a similar synthetic procedure as described in Example 1. LCMS: Method B, 3.698 min, MS: ES+ 236.4 (M+1); ¹H NMR (400 MHz, DMSO-d₆) δ ppm: 14.21 (br s, 1H), 7.20-7.25 (m, 2H), 7.07-7.18 (m, 2H), 4.06 (s, 2H), 2.10 (s, 3H).

### Example 5. Ethyl 5-methyl-4-(4-phenoxybenzyl)isoxazole-3-carboxylate (5)

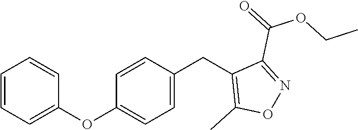 [SMILES: CCOC(=O)c1noc(C)c1Cc1ccc(Oc2ccccc2)cc1]

The title compound was synthesized via steps a, b, and c of Scheme 1, following similar synthetic procedures as described in Example 1, using 1-(bromomethyl)-4-phenoxybenzene in step b instead of 4-bromo benzyl bromide. LCMS: Method B, 4.994 min, MS: ES+ 338.5 (M+1); ¹H NMR (400 MHz, DMSO-d₆) δ ppm: 7.35-7.39 (m, 2H), 7.21-7.23 (m, 2H), 7.08-7.18 (m, 1H), 6.93-6.98 (m, 4H), 4.35-4.45 (m, 2H), 4.06 (s, 2H), 2.15 (s, 3H), 1.30 (t, J=6.8 Hz, 3H).

### Example 6. 5-Methyl-4-(4-phenoxybenzyl) isoxazole-3-carboxylic Acid (6)

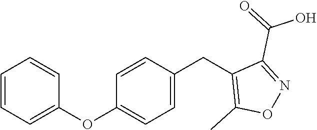 [SMILES: Cc1onc(C(=O)O)c1Cc1ccc(Oc2ccccc2)cc1]

The title compound was synthesized by hydrolysis of compound 5 ethyl 5-methyl-4-(4-phenoxybenzyl) isoxazole-3-carboxylate following similar synthetic procedure as described in Example 1. LCMS: Method A, 2.226 min, MS: ES− 308.2 (M−1); ¹H NMR (400 MHz, DMSO-d₆) δ ppm: 14.27 (br s, 1H), 7.37 (t, J=7.6 Hz, 2H), 7.21 (d, J=8.4 Hz, 2H), 7.11 (t, J=7.2 Hz, 1H), 6.91-6.98 (m, 4H), 4.06 (s, 2H), 2.13 (s, 3H).

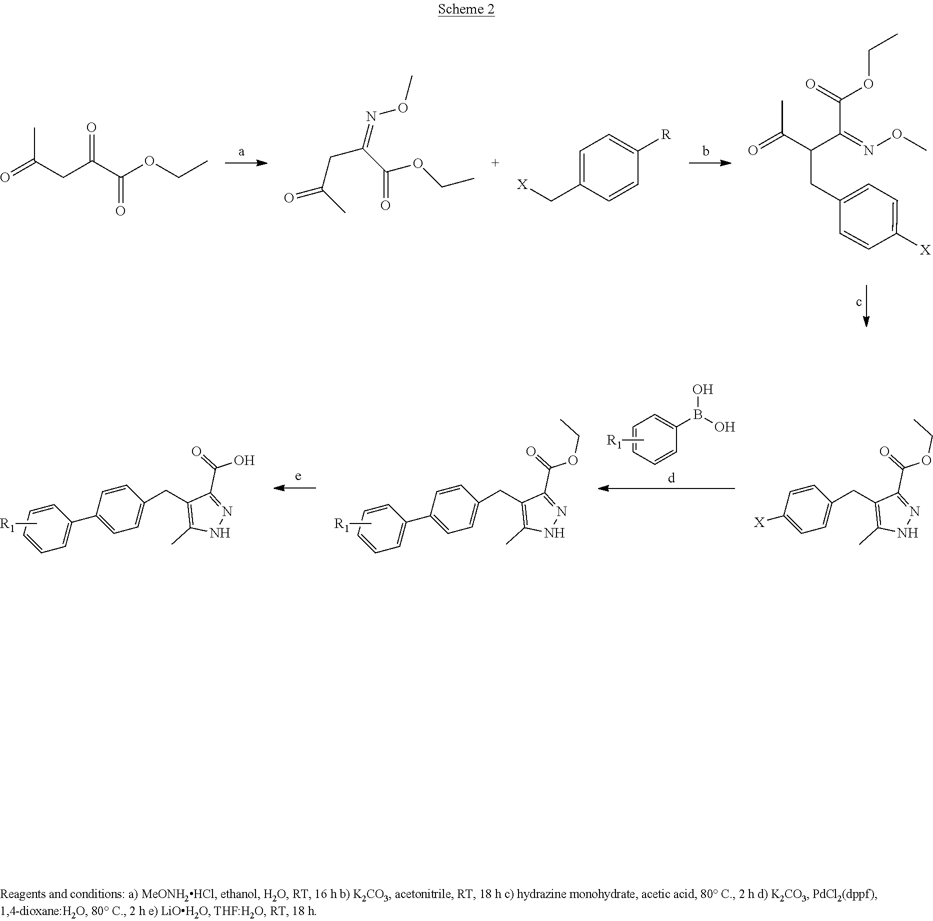

### Example 7. 4-((4′-Cyano-[1,1′-biphenyl]-4-yl)methyl)-5-methyl-H-pyrazole-3-carboxylic Acid (7)

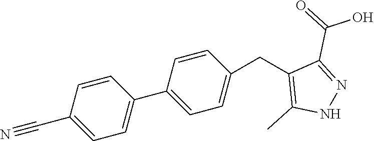 [SMILES: Cc1[nH]nc(C(=O)O)c1Cc1ccc(-c2ccc(C#N)cc2)cc1]

The title compound was prepared according to Scheme 2. To a stirred solution of ethyl (Z)-3-(4-bromobenzyl)-2-(methoxyimino)-4-oxopentanoate (compound 1.3; 0.5 g, 1.408 mmol) in acetic acid (5.0 mL) was added hydrazine hydrate (0.08 mL, 1.549 mmol) at ambient temperature. The reaction mixture was heated at 80° C. for 2 h. The resulting reaction mixture was cooled to ambient temperature and poured into saturated NaHCO₃ solution (50 mL). The resulting reaction mixture was extracted with EtOAc (2×50 mL). The combined organic phase was dried over Na₂SO₄, filtered and concentrated under reduced pressure. The resulting crude material was purified by flash chromatography (25.7% EtOAc in hexane) yielding ethyl 4-(4-bromobenzyl)-5-methyl-1H-pyrazole-3-carboxylate (compound 7.2; 0.3 g, 0.931 mmol). LCMS: Method B, 4.805 min, MS: ES+ 325.3 (M+1).

To a stirred solution of ethyl 4-(4-bromobenzyl)-5-methyl-1H-pyrazole-3-carboxylate (compound 7.2; 0.15 g, 0.465 mmol) in 1, 4-dioxane:water (2:1, 10 mL) were added K₂CO₃ (0.2 g, 1.397 mmol) and 4-cyano phenyl boronic acid (0.1 g, 0.698 mmol) at ambient temperature. The reaction mixture was purged with N₂ gas at ambient temperature for 15 minutes. PdCl₂(dppf) (0.03 g, 0.046 mmol) was added to the reaction mixture and heating was applied. The reaction mixture was stirred at 80° C. for 2 hours. The resulting reaction mixture was cooled to ambient temperature and poured into water (50 mL), extracted with EtOAc (2×20 mL). The combined organic phase was dried over Na₂SO₄, filtered and concentrated under reduced pressure. The resulting crude material was purified by flash chromatography (22.7% EtOAc in hexane) yielding ethyl 4-((4′-cyano-[1,1′-biphenyl]-4-yl)methyl)-5-methyl-1H-pyrazole-3-carboxylate (compound 7.1; 0.1 g, 0.289 mmol). LCMS: Method B, 4.694 min, MS: ES+ 346.5 (M+1); ¹H NMR (400 MHz, DMSO-d6) δ ppm: 13.16-13.45 (m, 1H), 7.88-7.90 (m, 2H), 7.82-7.84 (m, 2H), 7.62-7.66 (m, 2H), 7.24-7.28 (m, 2H), 4.06-4.20 (m, 2H), 4.06-4.08 (m, 2H), 2.12-2.20 (m, 3H), 1.20-1.28 (m, 3H).

To a stirred solution of ethyl 4-((4′-cyano-[1,1′-biphenyl]-4-yl)methyl)-5-methyl-1H-pyrazole-3-carboxylate (compound 7.1; 0.1 g, 0.289 mmol) in THF:water (2:1, 10 mL) was added LiOH.H₂O (0.02 g, 0.318 mmol) at ambient temperature and the reaction mixture was stirred for 18 h. The resulting reaction mixture was poured into saturated NaHCO₃ solution (20 mL) and washed with EtOAc (2×10 mL). The obtained aqueous layer was acidified (pH=4) with 0.1 N HCl and extracted with EtOAc (2×10 mL). The combined organic phase was dried over Na₂SO₄, filtered and concentrated under reduced pressure. The resulting solid was triturated with n-pentane (2×3 mL) and dried yielding 4-((4′-cyano-[1,1′-biphenyl]-4-yl)methyl)-5-methyl-1H-pyrazole-3-carboxylic acid (compound 7; 0.02 g, 0.063 mmol). LCMS: Method B, 4.090 min, MS: ES+ 318.5 (M+1); ¹H NMR (400 MHz, DMSO-d₆) δ ppm: 12.07 (br s, 1H), 7.90 (d, J=8.4 Hz, 2H), 7.83 (d, J=8.0 Hz, 2H), 7.63 (d, J=8.0 Hz, 2H), 7.26 (d, J=8.0 Hz, 2H), 4.03 (s, 2H), 2.13 (s, 3H).

### Example 8. 4-(4-Bromobenzyl)-5-methyl-1H-pyrazole-3-carboxylic Acid (8)

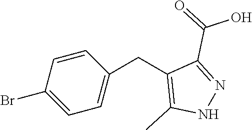 [SMILES: Cc1[nH]nc(C(=O)O)c1Cc1ccc(Br)cc1]

The title compound was synthesized by hydrolysis of compound 7.2 following the procedure described in Example 7. LCMS: Method B, 4.084 min, MS: ES+ 295.4 (M+1); ¹H NMR (400 MHz, DMSO-d₆) δ ppm: 7.42 (d, J=8.4 Hz, 2H), 7.08 (d, J=8.4 Hz, 2H), 3.98 (s, 2H), 2.09 (s, 3H).

### Example 9. Ethyl 5-methyl-4-(4-phenoxybenzyl)-1H-pyrazole-3-carboxylate (9)

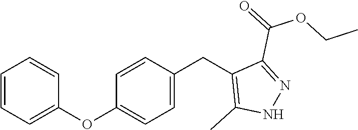 [SMILES: CCOC(=O)c1n[nH]c(C)c1Cc1ccc(Oc2ccccc2)cc1]

The title compound was synthesized via steps a, b, and c of Scheme 2, following the procedure described in Example 7 using 1-(bromomethyl)-4-phenoxybenzene in step b instead of 4-bromo benzyl bromide. LCMS: Method A, 2.417 min, MS: ES+ 337.3 (M+1); ¹H NMR (400 MHz, DMSO-d₆) δ ppm: 13.12-13.41 (m, 1H), 7.33-7.37 (m, 2H), 7.08-7.14 (m, 3H), 6.88-6.95 (m, 4H), 4.18-4.29 (m, 2H), 3.98 (s, 1H), 2.10-2.18 (m, 3H), 1.20-1.26 (m, 3H).

### Example 10. 5-Methyl-4-(4-phenoxybenzyl)-1H-pyrazole-3-carboxylic Acid (10)

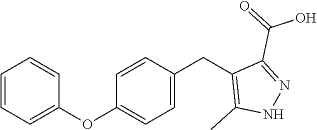 [SMILES: Cc1[nH]nc(C(=O)O)c1Cc1ccc(Oc2ccccc2)cc1]

The title compound was synthesized by hydrolysis of compound 9, ethyl 5-methyl-4-(4-phenoxybenzyl)-1H-pyrazole-3-carboxylate, following the procedure described in Example 7. LCMS: Method A, 2.101 min, MS: ES+ 263.2 (M+1); ¹H NMR (400 MHz, DMSO-d₆) δ ppm: 12.96 (br s, 1H), 7.30-7.40 (m, 2H), 7.09-7.16 (m, 3H), 6.88-6.95 (m, 4H), 3.99 (s, 2H), 2.11 (s, 3H).

### Example 11. Ethyl 4-(4-fluorobenzyl)-5-methyl-H-pyrazole-3-carboxylate (11)

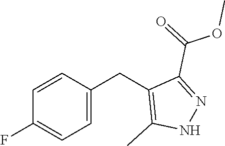 [SMILES: COC(=O)c1n[nH]c(C)c1Cc1ccc(F)cc1]

The title compound was synthesized via steps a, b, and c of Scheme 2, following the procedure described in Example 7 using 4-fluoro benzyl bromide in step b instead of 4-bromo benzyl bromide. LCMS: Method A, 2.089 min, MS: ES+ 263.19 (M+1); ¹H NMR (400 MHz, DMSO-d₆) δ ppm: 13.13-13.42 (m, 1H), 7.12-7.15 (m, 2H), 7.03-7.09 (m, 2H), 4.17-4.30 (m, 2H), 3.97-3.99 (m, 1H), 2.08-2.16 (m, 3H), 1.19-1.27 (m, 3H).

### Example 12. 5-Methyl-4-(4-fluorobenzyl) 1H-pyrazole-3-carboxylic Acid (12)

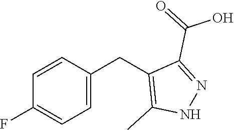 [SMILES: Cc1[nH]nc(C(=O)O)c1Cc1ccc(F)cc1]

The title compound was synthesized by hydrolysis of compound 11, ethyl 4-(4-fluorobenzyl)-5-methyl-1H-pyrazole-3-carboxylate, using the procedure described in Example 7. LCMS: Method A, 1.758 min, MS: ES+ 235.2 (M+1); H NMR (400 MHz, DMSO-d₆) δ ppm: 12.95 (br s, 1H), 7.14-7.17 (m, 2H), 7.05 (t, J=9.2 Hz, 2H), 3.99 (s, 2H), 2.09 (s, 3H).

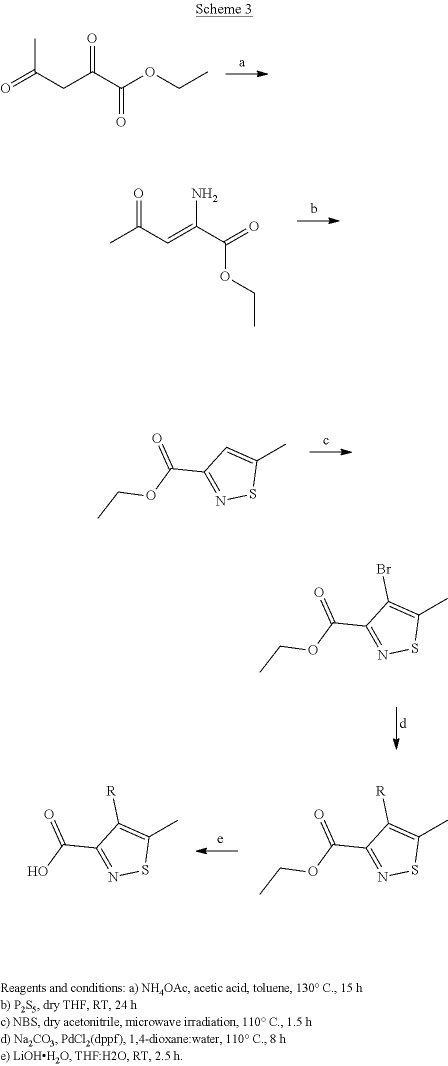

### Example 13. 4-(4-Fluorobenzyl)-5-methylisothiazole-3-carboxylic Acid (13)

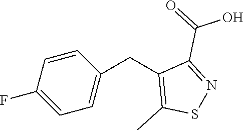 [SMILES: Cc1snc(C(=O)O)c1Cc1ccc(F)cc1]

The title compound was prepared according to Scheme 3. In a Dean-Stark assembly a stirred solution of ethyl 2, 4-dioxopentanoate (5 g, 31.605 mmol), acetic acid (1.4 mL) and NH₄OAc (4.87 g, 63.210 mmol) in dry toluene (100 mL) was prepared at ambient temperature. The reaction mixture was heated at 130° C. for 15 h with continuous removal of water. The resulting reaction mixture was cooled at ambient temperature, poured in to the NaHCO₃ solution (150 mL) and extracted with EtOAc (2×100 mL). The combined organic phase was dried over Na₂SO₄, filtered and concentrated under reduced pressure. The resulting crude material was purified by column chromatography (15% EtOAc in hexane) yielding ethyl (Z)-2-amino-4-oxopent-2-enoate (intermediate B; 2.97 g, 18.896 mmol). LCMS: Method A, 1.513 min, MS: ES+ 158.15 (M+1).

To a solution of ethyl (Z)-2-amino-4-oxopent-2-enoate (intermediate B; 2.9 g, 18.463 mmol) in THF (100 mL) was added P₂S5 (2.05 g, 9.231 mmol) at ambient temperature and the reaction mixture was stirred for 24 h. The resulting reaction mixture was evaporated to dryness and obtained residue was suspended in diethyl ether (100 mL). 30% hydrogen peroxide (5 mL) was added to the reaction mixture at ambient temperature and it was stirred for 10 minutes. Activated charcoal (0.3 g) was added to the reaction mixture and it was stirred for 20 minutes. The resulting reaction mixture was filtered through celite and the filtrate was washed with sodium hydrogen sulphite solution (2×50 mL). The organic layer was washed with brine (2×70 mL), dried over Na₂SO₄ and concentrated under reduced pressure. The resulting crude material was purified by column chromatography (18% EtOAc in hexane) yielding ethyl 5-methylisothiazole-3-carboxylate (intermediate C; 1.17 g, 6.839 mmol). LCMS: Method A, 1.666 min, MS: ES+ 172.07 (M+1).

To a stirred solution of ethyl 5-methylisothiazole-3-carboxylate (intermediate C; 0.5 g, 2.92 mmol) in dry acetonitrile (20 mL) was added NBS (5.2 g, 29.23 mmol) at ambient temperature. The reaction mixture was subjected to microwave irradiation and heated at 110° C. for 1.5 h. The resulting reaction mixture allowed to cool to ambient temperature, poured into water (100 mL) and extracted with EtOAc (2×75 mL). The combined organic phase was dried over Na₂SO₄, filtered and concentrated under reduced pressure. The resulting crude material was purified by flash chromatography (5% EtOAc in hexane) yielding ethyl 4-bromo-5-methylisothiazole-3-carboxylate (intermediate D; 0.7 g, 2.41 mmol). LCMS: Method A, 2.074 min, MS: ES+ 249.9 (M+1).

To a stirred solution of potassium trifluoro[(4-fluorophenyl)methyl]boranuide (CAS No. 1494466-28-2, 0.65 g, 3.015 mmol) and ethyl 4-bromo-5-methylisothiazole-3-carboxylate (intermediate D; 0.5 g, 2.01 mmol) in 1, 4-dioxane:water (4:1, 20 mL) was added Na₂CO₃ (0.65 g, 6.03 mmol) at ambient temperature. The reaction mixture was purged with N₂ gas at ambient temperature for 15 minutes. PdCl₂(dppf) (0.2 g, 0.2 mmol) was added to the reaction mixture and heating was applied. The reaction mixture was heated at 110° C. for 8 h. The resulting reaction mixture was cooled to ambient temperature, poured into water (20 mL) and extracted with EtOAc (2×20 mL). The combined organic phase was dried over Na₂SO₄, filtered and concentrated under reduced pressure. The resulting crude material was purified by flash chromatography (16% EtOAc in hexane) yielding ethyl 4-(4-fluorobenzyl)-5-methylisothiazole-3-carboxylate (compound 13.1; 0.06 g, 0.214 mmol). LCMS: Method A, 2.560 min, MS: ES+ 280.1 (M+1); ¹H NMR (400 MHz, DMSO-d₆) δ ppm: 7.05-7.13 (m, 4H), 4.25 (q, J=7.2, 14.4 Hz, 2H), 4.21 (s, 2H), 2.48 (s, 3H), 1.23 (t, J=7.2 Hz, 3H).

To a stirred solution of ethyl 4-(4-fluorobenzyl)-5-methylisothiazole-3-carboxylate (0.035 g, 0.125 mmol) in THF:water (8:2, 1 mL) was added LiOH.H₂O (0.005 g, 0.125 mmol) at room temperature and the resulting reaction mixture was stirred for 2.5 h. The resulting reaction mixture was poured into water (10 mL) and washed with EtOAc (2×15 mL). The aqueous layer was acidified (pH=4) with dilute citric acid and extracted with EtOAc (2×20 mL). The combined organic phase was dried over Na₂SO₄, filtered and concentrated under reduced pressure. The resulting solid was triturated with n-pentane:diethyl ether (1:1, 6 mL) and dried yielding 4-(4-fluorobenzyl)-5-methylisothiazole-3-carboxylic acid (compound 13; 0.015 g, 0.060 mmol). LCMS: Method B, 3.804 min, MS: ES+ 252.3 (M+1); ¹H NMR (400 MHz, DMSO-d₆) δ ppm: 13.37 (br s, 1H), 7.05-7.14 (m, 4H), 4.23 (s, 2H), 2.44 (s, 3H).

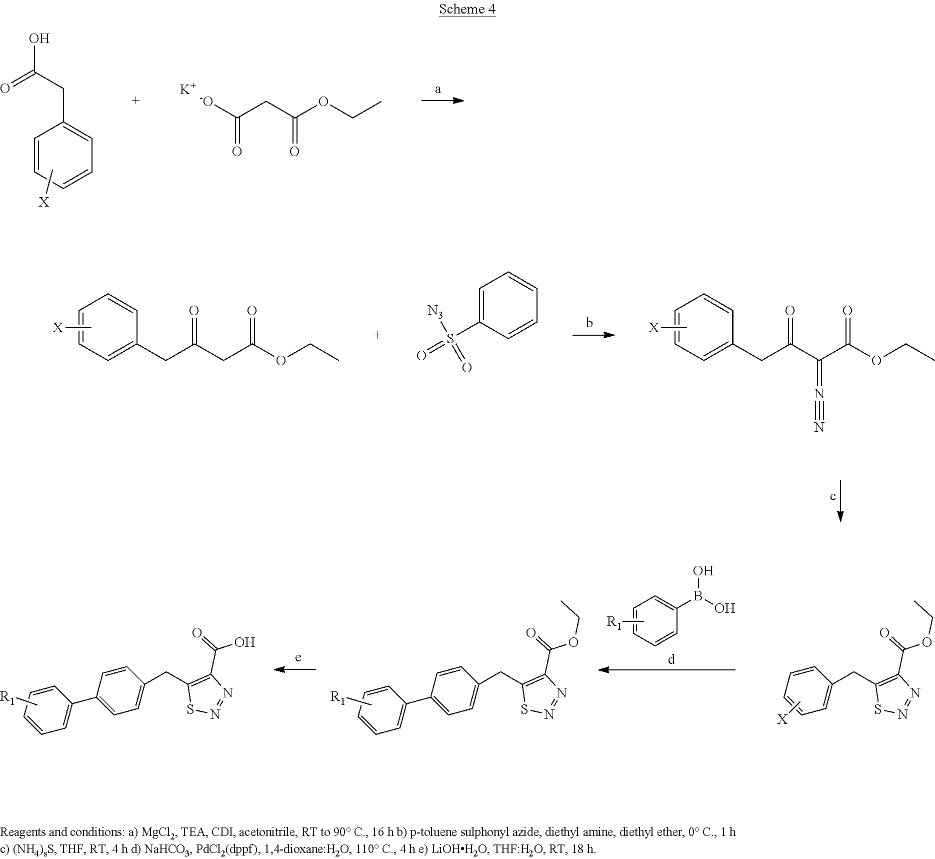

### Example 14. 5-((4′-Cyano-[1,1′-biphenyl]-4-yl)methyl)-1,2,3-thiadiazole-4-carboxylic Acid (14)

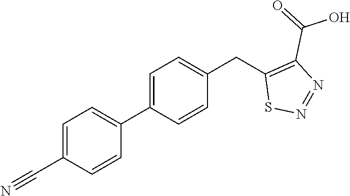 [SMILES: N#Cc1ccc(-c2ccc(Cc3snnc3C(=O)O)cc2)cc1]

### Step a

The title compound was prepared according to Scheme 4. To a stirred solution of 4-bromophenyl acetic acid (CAS Number 1878-68-8, 10.0 g, 46.511 mmol) in acetonitrile (100 mL) was added N,N′-carbonyldiimidazole (9.43 g, 58.139 mmol) at ambient temperature under nitrogen atmosphere and the reaction mixture was stirred for 16 h (Reaction mixture 1).

Separately, to a stirred solution of potassium 3-ethoxy-3-oxopropanoate (CAS Number 6148-64-7, 19.79 g, 116.277 mmol) in acetonitrile (100 mL) were added MgCl₂ (13.29 g, 139.533 mmol) and TEA (18 mL, 244.183 mmol) at 0° C. under nitrogen atmosphere and the reaction mixture was stirred at ambient temperature for 16 h (Reaction mixture 2).

Reaction mixture 1 was added slowly to the reaction mixture 2 at ambient temperature and heating was applied. The reaction mixture was heated at 90° C. for 3 hours. The resulting reaction mixture was cooled to ambient temperature, acidified using 2N HCl (150 mL) and extracted with EtOAc (2×100 mL). The combined organic phase was dried over Na₂SO₄, filtered and concentrated under reduced pressure yielding ethyl 4-(4-bromophenyl)-3-oxobutanoate (compound 14.4; 11.7 g, 41.032 mmol). LCMS: Method B, 4.544 min, MS: ES+ 285.3 (M+1).

To a stirred solution of ethyl 4-(4-bromophenyl)-3-oxobutanoate (compound 14.4; 1.0 g, 3.507 mmol) in diethyl ether (5 mL) were added p-toluene sulfonyl azide (0.7 g, 3.507 mmol) and diethyl amine (0.17 g, 2.279 mmol) at 0° C. The reaction mixture was stirred at 0° C. for 1 hour. The resulting reaction mixture was poured into water (100 mL) and extracted with EtOAc (2×50 mL). The combined organic phase was dried over Na₂SO₄, filtered and concentrated under reduced pressure yielding ethyl 4-(4-bromophenyl)-2-diazo-3-oxobutanoate (compound 14.3; 1.46 g, quantitative). LCMS: Method B, 4.865 min, MS: ES+311.3 (M+1).

To a stirred solution of ethyl 4-(4-bromophenyl)-2-diazo-3-oxobutanoate (compound 14.3; 2.3 g, 7.392 mmol) in THF (5 mL) was added (NH₄)₂S (2.41 g, 35.335 mmol) at 0° C. The reaction mixture was stirred at ambient temperature for 4 hours. The resulting reaction mixture was poured into water (100 mL) and extracted with EtOAc (2×50 mL). The combined organic phase was dried over Na₂SO₄, filtered and concentrated under reduced pressure. The resulting crude material was purified by flash chromatography (40% EtOAc in hexane) yielding ethyl 5-(4-bromobenzyl)-1, 2, 3-thiadiazole-4-carboxylate (compound 14.2; 0.87 g, 2.668 mmol). LCMS: Method B, 4.798 min, MS: ES+ 327.3 (M+1).

To a stirred solution of ethyl 5-(4-bromobenzyl)-1, 2, 3-thiadiazole-4-carboxylate (compound 14.2; 0.3 g, 0.920 mmol) in 1, 4-dioxane:water (2:1, 10 mL) were added NaHCO₃ (0.2 g, 2.760 mmol) and 4-cyano phenyl boronic acid (CAS No. 126747-14-6, 0.2 g, 1.380 mmol) at ambient temperature. The reaction mixture was purged with N₂ gas at ambient temperature for 15 minutes. PdCl₂(dppf) (0.07 g, 0.092 mmol) was added to the reaction mixture at ambient temperature and heating was applied. The reaction mixture was stirred at 110° C. for 4 hours. The resulting reaction mixture was cooled to ambient temperature, poured into water (100 mL) and extracted with EtOAc (2×30 mL). The combined organic phase was dried over Na₂SO₄, filtered and concentrated under reduced pressure. The resulting crude material was purified by flash chromatography (30% EtOAc in hexane) yielding ethyl 5-((4′-cyano-[1,1′-biphenyl]-4-yl)methyl)-1,2,3-thiadiazole-4-carboxylate (compound 14.1; 0.19 g, 0.544 mmol). LCMS: Method B, 4.681 min, MS: ES+350.4 (M+1); ¹H NMR (400 MHz, DMSO-d₆) δ ppm: 7.88-7.94 (m, 4H), 7.76 (d, J=8.4 Hz, 2H), 7.48 (d, J=8.0 Hz, 2H), 4.75 (s, 2H), 4.45 (q, J=7.2, 14.4 Hz, 2H), 1.37 (t, J=7.2 Hz, 3H).

To a stirred solution of ethyl 5-((4′-cyano-[1,1′-biphenyl]-4-yl)methyl)-1,2,3-thiadiazole-4-carboxylate (compound 14.1; 0.15 g, 0.429 mmol) in THF:water (2:1, 12 mL) was added LiOH.H₂O (0.055 g, 1.287 mmol) at 0° C. The reaction mixture was stirred at ambient temperature for 18 h. The resulting reaction mixture was poured into saturated NaHCO₃ solution (20 mL) and washed with EtOAc (2×10 mL). The obtained aqueous layer was acidified (pH=4) with 0.1 N HCl and extracted with EtOAc (2×10 mL). The combined organic phase was dried over Na₂SO₄, filtered and concentrated under reduced pressure. The resulting crude was purified by Prep HPLC yielding 5-((4′-cyano-[1,1′-biphenyl]-4-yl)methyl)-1,2,3-thiadiazole-4-carboxylic acid (compound 14; 0.011 g, 0.034 mmol). LCMS: Method B, 3.912 min, MS: ES+ 322.3 (M+1); ¹H NMR (400 MHz, DMSO-d₆) δ ppm: 7.87-7.94 (m, 4H), 7.73 (d, J=8.4 Hz, 2H), 7.48 (d, J=8.4 Hz, 2H), 4.76 (s, 2H).

### Example 15. 5-(4-Bromobenzyl)-1, 2, 3-thiadiazole-4-carboxylic Acid (15)

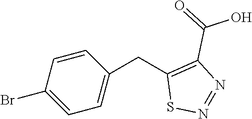 [SMILES: O=C(O)c1nnsc1Cc1ccc(Br)cc1]

The title compound was synthesized by hydrolysis of compound 14.2 following the procedure described in Example 13. LCMS: Method B, 3.843 min, MS: ES+ 299.2 (M+1); ¹H NMR (400 MHz, DMSO-d₆) δ ppm: 14.01 (br s, 1H), 7.55 (d, J=8.4 Hz, 2H), 7.31 (d, J=8.4 Hz, 2H), 4.66 (s, 2H).

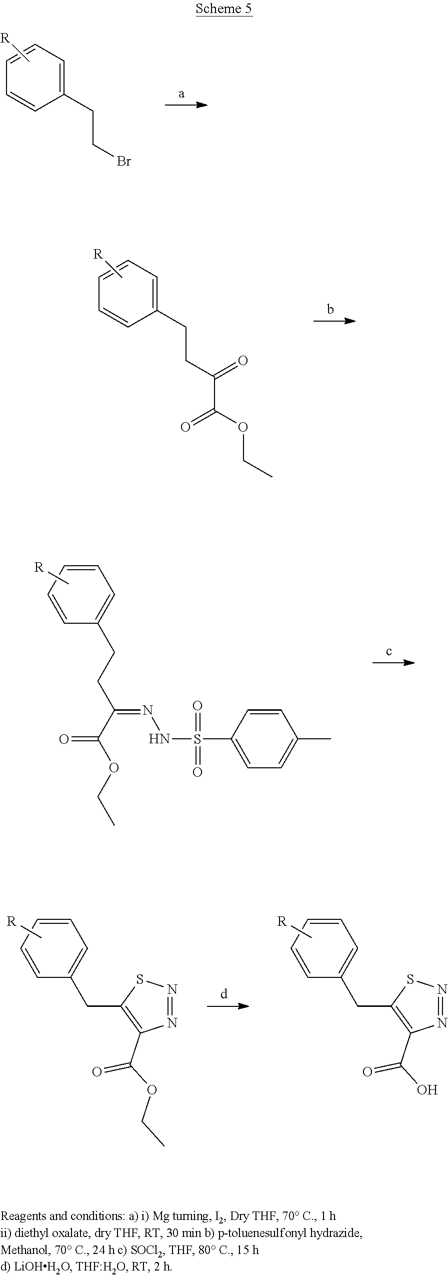

### Example 16. 5-(4-Fluorobenzyl)-1,2,3-thiadiazole-4-carboxylic Acid (16)

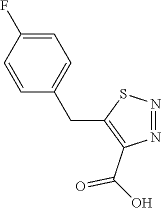 [SMILES: O=C(O)c1nnsc1Cc1ccc(F)cc1]

The title compound 16 was prepared according to Scheme 5. Magnesium turnings (0.32 g, 0.013 mmol) were taken in dry THF (40 mL) and 12 (catalytic amount) was added under nitrogen atmosphere at ambient temperature. A solution of 4-fluoro-(2-bromoethyl) benzene (2.5 g, 12.312 mmol) in dry THF (20 mL) was added drop wise to the above mixture at 70° C. The resulting reaction mixture was heated at 70° C. for 1 h and then cooled to ambient temperature. Separately, a solution of diethyl oxalate (2.16 g, 14.774 mmol) in dry THF (20 mL) was prepared and cooled to −10° C. The previously prepared Grignard reagent was added to the solution of diethyl oxalate at −10° C. under nitrogen atmosphere. The reaction mixture was stirred at ambient temperature for 30 min. The resulting reaction mixture was poured into water (150 mL) and extracted with EtOAc (2×100 mL). The combined organic phase was dried over Na₂SO₄, filtered and concentrated under reduced pressure. The resulting crude material was purified by flash chromatography (15% EtOAc in hexane) yielding ethyl 4-(4-fluorophenyl)-2-oxobutanoate (compound 16.3; 1.01 g, 4.507 mmol). LCMS: Method B, 4.476 min, MS: ES+ 242.5 (M+18).

A solution of ethyl 4-(4-fluorophenyl)-2-oxobutanoate (compound 16.3; 0.7 g, 3.124 mmol) and p-toluene sulfonyl hydrazide (0.58 g, 3.124 mmol) in methanol (15 mL) was heated at 70° C. for 24 h. The resulting reaction mixture was concentrated under vacuum. The obtained residue was suspended in water (70 mL) and extracted with EtOAc (2×50 mL). The combined organic phase was dried over Na₂SO₄, filtered and concentrated under reduced pressure. The resulting crude material was purified by flash chromatography (15% EtOAc in hexane) yielding ethyl (Z)-4-(4-fluorophenyl)-2-(2-tosylhydrazono)butanoate (compound 16.2; 0.79 g, 2.014 mmol). LCMS: Method A, 2.074 min, MS: ES+ 393.35 (M+1).

To a stirred solution of ethyl (Z)-4-(4-fluorophenyl)-2-(2-tosylhydrazono)butanoate (compound 16.2; 0.34 g, 0.867 mmol) in dry THF (10 mL) was added SOCl₂ (2.06 g, 17.34 mmol) at ambient temperature. The reaction mixture was heated at 80° C. for 15 h. The resulting reaction mixture was cooled to ambient temperature and poured into water (10 mL). The resulting reaction mixture was neutralized (pH=8) with a saturated NaHCO₃ solution and extracted with EtOAc (2×20 mL). The combined organic phase was dried over Na₂SO₄, filtered and concentrated under reduced pressure. The resulting crude material was purified by prep HPLC (using water/acetonitrile/NH₄HCO₃ as buffer) yielding ethyl 5-(4-fluorobenzyl)-1,2,3-thiadiazole-4-carboxylate (compound 16.1; 0.028 g, 0.105 mmol). LCMS: Method A, 2.285 min, MS: ES+ 267.10 (M+1); ¹H NMR (400 MHz, DMSO) δ ppm: 7.42-7.38 (m, 2H), 7.19 (t, J=8.8 Hz, 2H), 4.66 (s, 2H), 4.46-4.40 (q, J=6.8, 14.0 Hz, 2H) 1.36 (t, J=4.0 Hz, 3H).

To a stirred solution of ethyl 5-(4-fluorobenzyl)-1,2,3-thiadiazole-4-carboxylate (compound 16.1; 0.02 g, 0.075 mmol) in THF:water (4:1, 1.5 mL) was added LiOH.H₂O (0.003 g, 0.075 mmol) at ambient temperature and the reaction mixture was stirred for 2 h. The resulting reaction mixture was poured into water (10 mL) and washed with EtOAc (2×15 mL). The aqueous layer was then acidified (pH=4) with KHSO₄ and extracted with EtOAc (2×10 mL). The combined organic phase was dried over Na₂SO₄, filtered and concentrated under reduced pressure. The resulting solid was triturated with n-pentane:diethyl ether (1:1, 6 mL) and dried yielding 5-(4-fluorobenzyl)-1,2,3-thiadiazole-4-carboxylic acid (compound 16; 0.008 g, 0.033 mmol). LCMS: Method B, 3.644 min, MS: ES+ 239.4 (M+1); ¹H NMR (400 MHz, MeOD) δ ppm: 7.42-7.38 (m, 2H), 7.18 (t, J=8.8 Hz, 2H), 4.66 (s, 2H).

### Example 17. Ethyl 5-(4-phenoxybenzyl)-1,2,3-thiadiazole-4-carboxylate (17)

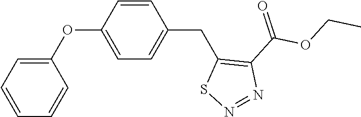 [SMILES: CCOC(=O)c1nnsc1Cc1ccc(Oc2ccccc2)cc1]

### Synthesis of 1-(2-bromoethyl)-4-phenoxybenzene (Intermediate E)

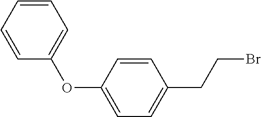 [SMILES: BrCCc1ccc(Oc2ccccc2)cc1]

To a suspension of methyl triphenylphosphonium bromide (CAS Number 1779-49-3, 19.84 g, 55.5 mmol) in THF (50 mL) was added potassium tert-butoxide (CAS Number 865-47-4, 10.2 g, 90.9 mmol) at ambient temperature and the reaction mixture was stirred for 30 minutes. 4-phenoxybenzaldehyde (CAS Number 67-36-7, 10.0 g, 50.5 mmol) was added to the reaction mixture at ambient temperature and stirred for 15 h. The resulting reaction mixture was diluted with n-hexane (100 mL) and stirred for 40 min. The obtained suspension was filtered through silica gel (60-120 mesh size) and the filtrate was evaporated under reduced pressure yielding 1-phenoxy-4-vinylbenzene (9.4 g, 47.94 mmol). ¹H NMR (400 MHz, CDCl₃) δ ppm: 7.34-7.42 (m, 4H), 7.11-7.15 (m, 1H), 6.97-7.054 (m, 4H), 6.68-6.75 (m, 1H), 5.69 (dd, J=0.8, 17.6 Hz, 1H), 5.22 (dd, J=0.8, 11.2 Hz, 1H).

To a stirred solution of 1-phenoxy-4-vinylbenzene (9.4 g, 47.94 mmol) in THF was added borane dimethylsulfide complex (2M in THF, CAS Number 13292-87-0, 3.60 g, 47.43 mmol) drop wise at 0° C. The reaction mixture was stirred at ambient temperature for 3 h and then cooled to 0° C. Ethanol (11.6 mL), 15% NaOH (10 mL) and 30% H₂O₂ solution (10 mL) were added subsequently in to the reaction mixture at 0° C. The resulting reaction mixture was heated at 100° C. for 2 h and then cooled to ambient temperature. Water (150 mL) was added to the reaction mixture and extracted with diethyl ether (3×150 mL). The combined organic layer was dried over anhydrous Na₂SO₄, filtered and evaporated under reduced pressure. The resulting crude material was purified by column chromatography (15% EtOAc in hexane) yielding 2-(4-phenoxyphenyl)ethan-1-ol (5.7 g, 26.62 mmol). ¹H NMR (400 MHz, DMSO-d₆) δ ppm: 7.35-7.39 (m, 2H), 7.23 (d, J=8.4 Hz, 2H), 7.11 (d, J=7.6 Hz, 1H), 6.97 (dd, J=0.8, 8.4 Hz, 2H), 6.92 (dd, J=2.0, 6.8 Hz, 2H), 4.65 (t, J=5.2 Hz, 1H), 3.57-3.62 (m, 2H), 2.70 (t, J=6.8 Hz, 2H).

To stirred solution of 2-(4-phenoxyphenyl)ethan-1-ol (5.7 g, 26.62 mmol) in dichloromethane (50 mL) were added triphenylphosphene (CAS Number 603-35-0, 13.97 g, 53.25 mmol) and imidazole (CAS Number 288-32-4, 3.62 g, 53.25 mmol) at ambient temperature. The resulting mixture was cooled to 0° C. and carbon tetrabromide (13.24 g, 39.94 mmol) was added to the reaction mixture in small portions over a period of 20 minutes. The reaction mixture was stirred at ambient temperature for 1 h. The resulting reaction mixture was evaporated to dryness and the obtained crude material was purified by column chromatography (100% hexane) yielding 1-(2-bromoethyl)-4-phenoxybenzene (intermediate E; 3.2 g, 11.59 mmol).

The title compound 17 was synthesized via steps a, b, and c of Scheme 5, following the procedure described in Example 16 using 1-(2-bromoethyl)-4-phenoxybenzene (intermediate E) in step a instead of 4-fluoro-(2-bromoethyl) benzene. LCMS: Method B, 4.971 min, MS: ES+ 341.4 (M+1); ¹H NMR (400 MHz, DMSO-d₆) δ ppm: 7.35-7.42 (m, 4H), 7.13-7.17 (m, 1H), 6.97-7.02 (m, 4H), 4.65 (s, 2H), 4.44 (q, J=7.2, 14.4 Hz, 2H), 1.36 (t, J=7.2 Hz, 3H).

### Example 18. 5-(4-Phenoxybenzyl)-1,2,3-thiadiazole-4-carboxylic Acid (18)

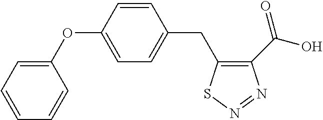 [SMILES: O=C(O)c1nnsc1Cc1ccc(Oc2ccccc2)cc1]

The title compound was synthesized by hydrolysis of compound 17 ethyl 5-(4-phenoxybenzyl)-1,2,3-thiadiazole-4-carboxylate, following similar synthetic described in Example 16. LCMS: Method B, 4.298 min, MS: ES+ 313.3 (M+1); ¹H NMR (400 MHz, DMSO-d₆) δ ppm: 7.33-7.40 (m, 4H), 7.12 (t, J=7.2 Hz, 1H), 6.98-7.02 (m, 2H), 6.94 (d, J=8.4 Hz, 2H), 4.68 (s, 2H).

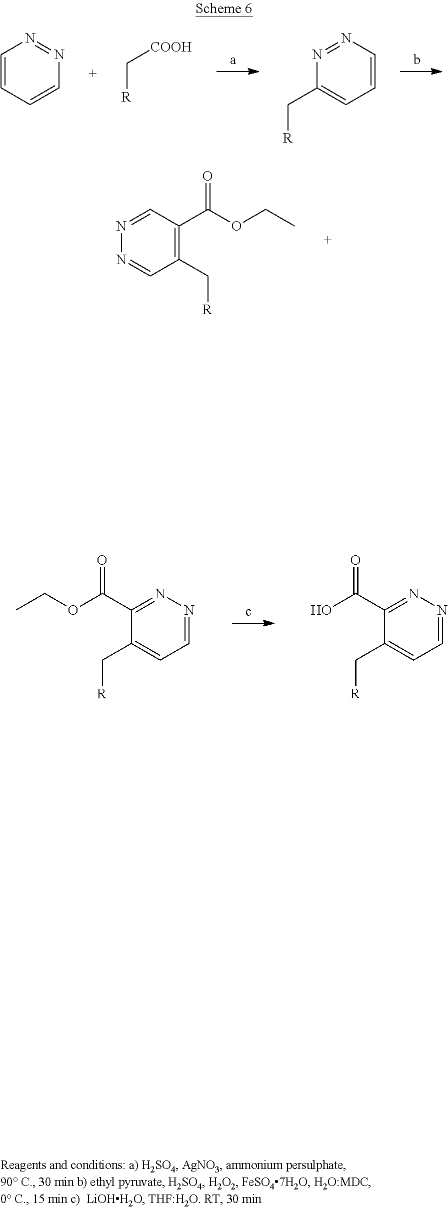

### Example 19. 4-(4-Fluorobenzyl) pyridazine-3-carboxylic Acid (19)

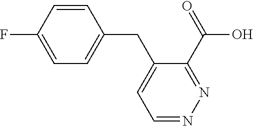 [SMILES: O=C(O)c1nnccc1Cc1ccc(F)cc1]

The title compound was prepared according to Scheme 6. To a stirred solution of pyridazine (CAS Number 289-80-5, 2.0 g, 24.971 mmol) in 2N H₂SO₄ (25 mL) were subsequently added AgNO₃ (1.3 g, 7.491 mmol) and 4-fluoro phenyl acetic acid (CAS Number 405-50-5, 3.8 g, 24.971 mmol) at 70° C. The reaction mixture was heated at 70° C. for 20 minutes followed by nitrogen purging for 5 minutes before addition of ammonium persulphate (17.0 g, 74.917 mmol) in small portions directly at 70° C. The resulting reaction mixture was further heated at 90° C. for 30 minutes and then cooled to ambient temperature. The obtained reaction mixture was poured into 50% NaOH (200 mL) and diluted with EtOAc (50 mL). The resulting mixture was filtered through celite and layers were separated. The resulting aqueous was further extracted with EtOAc (2×100 mL). The combined organic phase was dried over Na₂SO₄, filtered and concentrated under reduced pressure. The resulting crude material was purified by flash chromatography (1% Methanol in dichloromethane) yielding 4-(4-fluorobenzyl)pyridazine (compound 19.3; 1.2 g, 6.380 mmol) and. LCMS: Method B, 3.856 min, MS: ES+ 189.4 (M+1)

A mixture of H₂SO₄ (0.5 mL) was prepared in MDC:water (7:1, 8 mL) at −5° C. and added to a mixture of 4-(4-fluorobenzyl)pyridazine (compound 19.3; 0.58 g, 3.083 mmol) and FeSO₄.7H₂O at −5° C. (Reaction mixture 1). Separately, H₂O₂ (1.0 mL, 9.251 mmol) was added to ethyl pyruvate (1.5 mL, 13.877 mmol) at −5° C. and stirred for 15 minutes (Reaction mixture 2). Reaction mixture 2 was added drop wise in to the reaction mixture 1 at 0° C. and stirred for 15 minutes. The resulting reaction mixture was poured into saturated Na₂SO₃ solution (50 mL) and extracted with EtOAc (2×25 mL). The combined organic phase was dried over Na₂SO₄, filtered and concentrated under reduced pressure. The resulting crude material was purified by prep HPLC (using water/acetonitrile/NH₄HCO₃ as buffer) yielding ethyl 5-(4-fluorobenzyl)pyridazine-4-carboxylate (compound 19.2; 0.04 g, 0.153 mmol) LCMS: Method B, 4.390 min, MS: ES+ 261.4 (M+1); ¹H NMR (400 MHz, DMSO-d₆) δ ppm: 9.39 (d, J=0.8 Hz, 1H), 9.34 (s, 1H), 7.25-7.28 (m, 2H), 7.11-7.17 (m, 2H), 4.31-4.36 (m, 4H), 1.28 (t, J=7.2 Hz, 3H) and ethyl 4-(4-fluorobenzyl)pyridazine-3-carboxylate (compound 19.1; 0.025 g, 0.096 mmol). LCMS: Method B, 4.452 min, MS: ES+ 261.4 (M+1).

To a stirred solution of ethyl 4-(4-fluorobenzyl) pyridazine-3-carboxylate (compound 19.1; 0.02 g, 0.076 mmol) in THF:water (2:1, 1.2 mL) was added LiOH.H₂O (0.004 g, 0.084 mmol) at ambient temperature and the reaction mixture was stirred for 30 minutes. The resulting reaction mixture was poured into saturated NaHCO₃ solution (10 mL) and washed with EtOAc (2×5 mL). The obtained aqueous layer was acidified (pH=4) with dilute HCl and extracted with EtOAc (2×5 mL). The combined organic phase was dried over Na₂SO₄, filtered and concentrated under reduced pressure. The resulting crude was purified by prep HPLC (using acetonitrile/water/NH₄HCO₃) yielding 4-(4-fluorobenzyl)pyridazine-3-carboxylic acid (compound 19; 0.003 g, 0.012 mmol). LCMS: Method B, 3.124 min, MS: ES+ 233.5 (M+1); ¹H NMR (400 MHz, DMSO-d₆) δ ppm: 8.81 (d, J=5.2 Hz, 1H), 7.35-7.39 (m, 2H), 7.08-7.16 (m, 3H), 3.96 (s, 2H).

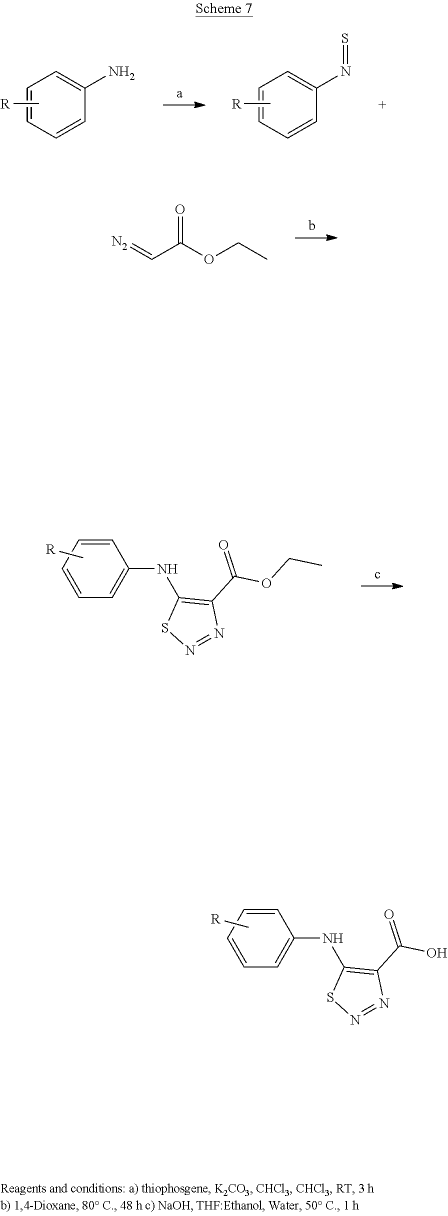

### Example 20. 5-((4-(1H-Pyrazol-1-yl)phenyl)amino)-1,2,3-thiadiazole-4-carboxylic Acid (20)

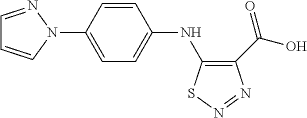 [SMILES: O=C(O)c1nnsc1Nc1ccc(-n2cccn2)cc1]

The title compound was prepared according to Scheme 7. To a stirred solution of 4-(1H-pyrazol-1-yl)aniline (CAS Number 17635-45-9, 0.5 g, 3.140 mmol) in chloroform (25 mL) was added K₂CO₃ (1.3 g, 9.422 mmol) at ambient temperature and the reaction mixture was cooled to 0-5° C. Thiophosgene (0.73 g, 6.281 mmol) was added the reaction mixture at 0-5° C. The resulting reaction mixture was stirred at ambient temperature for 3 h and then poured into water (15 mL). The obtained mixture was neutralized by NaHCO₃ and extracted with EtOAc (2×100 mL). The combined organic phase was dried over Na₂SO₄, filtered and concentrated under reduced pressure yielding 1-(4-isothiocyanatophenyl)-1H-pyrazole (compound 20.2; 0.56 g, 2.785 mmol). LCMS: Method A, 3.379 min, MS: ES+ 202.1 (M+1).

To a stirred solution of 1-(4-isothiocyanatophenyl)-1H-pyrazole (compound 20.2; 0.46 g, 2.288 mmol) in 1,4-dioxane (10 mL) was added ethyl diazoacetate solution (15% in toluene) (2.9 g, 3.432 mmol) at ambient temperature and the reaction mixture was heated at 80° C. for 48 h. The resulting reaction mixture was poured into water (100 mL) and extracted with EtOAc (2×50 mL). The combined organic phase was washed with brine (25 mL), dried over Na₂SO₄, filtered and concentrated under reduced pressure. The resulting crude material was purified by flash chromatography (10% EtOAc in hexane) yielding ethyl 5-((4-(1H-pyrazol-1-yl)phenyl)amino)-1,2,3-thiadiazole-4-carboxylate (compound 20.1; 0.01 g, 0.032 mmol). LCMS: Method A, 2.202 min, MS: ES+ 316.17 (M+1); ¹H NMR (400 MHz, DMSO-d₆) δ ppm: 10.19 (s, 1H), 8.53 (d, J=2.4 Hz, 1H), 7.93 (d, J=8.8 Hz, 2H), 7.76 (d, J=1.6 Hz, 1H), 7.57 (d, J=9.2 Hz, 2H), 6.57 (t, J=2.0 Hz, 1H), 4.45 (q, J=7.2, 14.0 Hz, 2H), 1.38 (t, J=7.2 Hz, 3H).

To a stirred solution of ethyl 5-((4-(1H-pyrazol-1-yl)phenyl)amino)-1,2,3-thiadiazole-4-carboxylate (compound 20.1; 0.011 g, 0.035 mmol) in THF:ethanol:water (1:1:1, 0.9 mL) was added LiOH.H₂O (0.003 g, 0.070 mmol) at ambient temperature and the reaction mixture was heated at 50° C. for 1 h. The resulting reaction mixture was poured into saturated NaHCO₃ solution (5 mL) and washed with EtOAc (2×10 mL). The obtained aqueous layer was acidified (pH=3) with 1N HCl and extracted with EtOAc (3×10 mL). The combined organic phase was dried over Na₂SO₄, filtered and concentrated under reduced pressure yielding 5-((4-(1H-pyrazol-1-yl)phenyl)amino)-1,2,3-thiadiazole-4-carboxylic acid (compound 20; 0.009 g, 0.031 mmol). LCMS: Method B, 2.737 min (88.68%) 3.738 min (2.41%), MS: ES+ 288.4 (M+1); ¹H NMR (400 MHz, DMSO-d₆) δ ppm: 13.48 (s, 1H), 8.55-8.62 (m, 1H), 7.95-8.07 (m, 4H), 7.78-7.83 (m, 1H), 6.58-6.62 (m, 1H).

### Example 21. Ethyl 5-((4-fluorophenyl)amino)-1,2,3-thiadiazole-4-carboxylate (21)

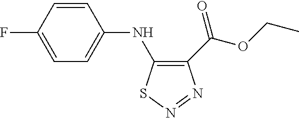 [SMILES: CCOC(=O)c1nnsc1Nc1ccc(F)cc1]

The title compound was synthesized via step b of Scheme 7, following the procedure described in Example 20, using 4-fluorophenyl isothiocyanate (CAS Number 1544-68-9) in step b instead of 1-(4-isothiocyanatophenyl)-1H-pyrazole. LCMS: Method A, 2.283 min, MS: ES+ 268.15 (M+1); ¹H NMR (400 MHz, DMSO-d₆) δ ppm: 10.07 (s, 1H), 7.49-7.52 (m, 2H), 7.29-7.33 (m, 2H), 4.43 (q, J=6.8, 14.0 Hz, 2H), 1.37 (t, J=7.2 Hz, 3H).

### Example 22. 5-((4-Fluorophenyl)amino)-1,2,3-thiadiazole-4-carboxylic Acid (22)

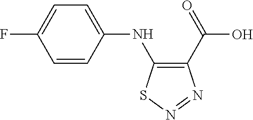 [SMILES: O=C(O)c1nnsc1Nc1ccc(F)cc1]

The title compound was synthesized by hydrolysis of compound 21, ethyl 5-((4-fluorophenyl)amino)-1,2,3-thiadiazole-4-carboxylate, following the procedure described in Example 20. LCMS: Method C, 3.830 min (4.06%), 4.109 min (92.59%), MS: ES+ 240.3 (M+1); ¹H NMR (400 MHz, DMSO-d₆) δ ppm: 13.44 (br s, 1H), 10.15 (s, 1H), 7.28-7.90 (m, 4H).

### Example 23. 5-((3-Bromophenyl)amino)-1,2,3-thiadiazole-4-carboxylic Acid (23)

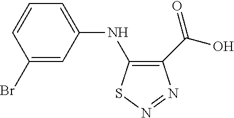 [SMILES: O=C(O)c1nnsc1Nc1cccc(Br)c1]

The title compound was synthesized via steps b and c of Scheme 7, following the procedure described in Example 20 using 3-bromophenyl isothiocyanate (CAS Number 2131-59-1) in step b instead of 1-(4-isothiocyanatophenyl)-1H-pyrazole. LCMS: Method B, 2.219 min (16.41%), 4.063 min (75.29%), MS: ES+ 300.3 (M+1); ¹H NMR (400 MHz, DMSO-d₆) δ ppm: 13.66 (br s, 1H), 10.28 (s, 1H), 7.40-8.26 (m, 4H).

### Example 24. 5-((4-Bromophenyl)amino)-1,2,3-thiadiazole-4-carboxylic Acid (24)

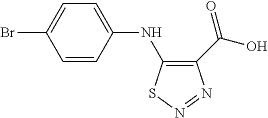 [SMILES: O=C(O)c1nnsc1Nc1ccc(Br)cc1]

The title compound was synthesized via steps b and c of Scheme 7, following the procedure described in Example 20 using 4-bromophenyl isothiocyanate (CAS Number 1985-12-2) in step b instead of 1-(4-isothiocyanatophenyl)-1H-pyrazole. LCMS: Method B, 4.081 min, MS: ES+ 300.4 (M+1); ¹H NMR (400 MHz, DMSO-d₆) δ ppm: 13.67 (br s, 1H), 10.29 (s, 1H), 7.63-7.66 (m, 2H), 7.38-7.42 (m, 2H).

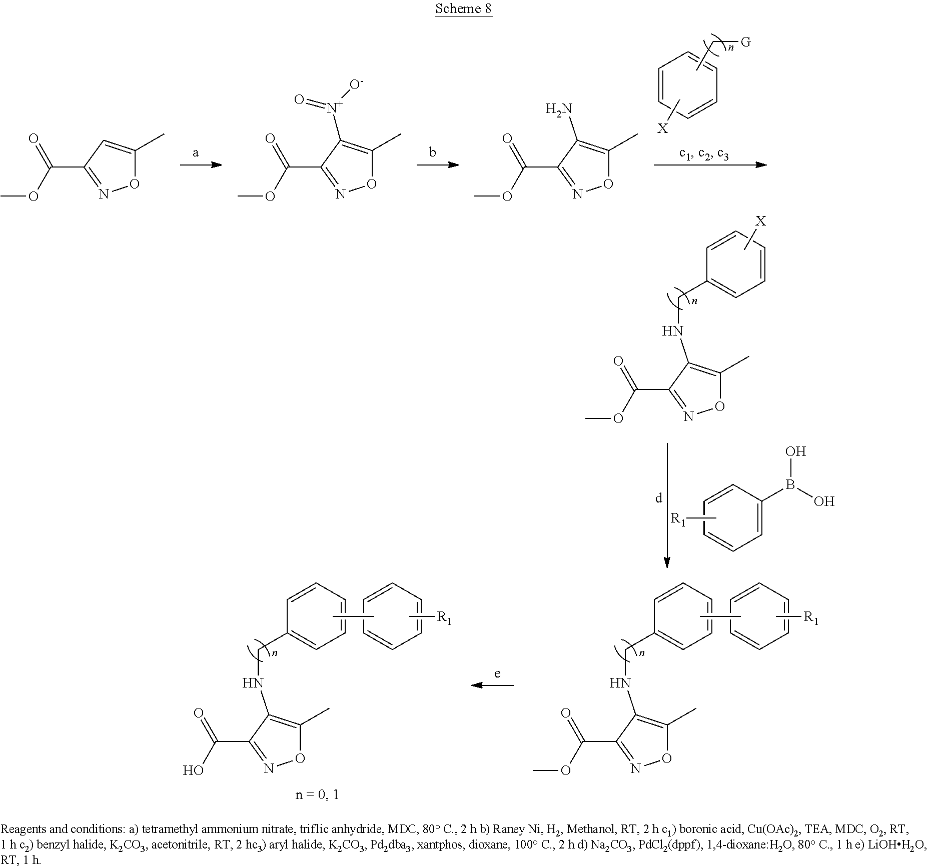

### Example 25. 4-((4′-Cyano-[1,1′-biphenyl]-4-yl)amino)-5-methylisoxazole-3-carboxylic acid

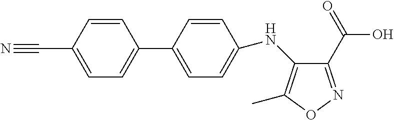 [SMILES: Cc1onc(C(=O)O)c1Nc1ccc(-c2ccc(C#N)cc2)cc1]

The title compound was prepared according to Scheme 8. To a suspension of tetra methyl ammonium nitrate (7.3 g, 53.153 mmol) in dichloromethane (20 mL) was added triflic anhydride (15.0 g, 53.153 mmol) drop wise at ambient temperature under nitrogen atmosphere. The resulting suspension was stirred at ambient temperature for 90 minutes followed by addition of methyl 5-methylisoxazole-3-carboxylate (CAS Number 19788-35-3, 5.0 g, 35.435 mmol). The reaction mixture was heated at 80° C. for 2 h under N₂ gas atmosphere. The resulting reaction mixture was cooled to ambient temperature, poured into saturated NaHCO₃ (200 mL) and extracted with EtOAc (2×100 mL). The combined organic phase was dried over Na₂SO₄, filtered and concentrated under reduced pressure. The resulting residue was purified by column chromatography (10% EtOAc in hexane) yielding methyl 5-methyl-4-nitroisoxazole-3-carboxylate (compound 25.4; 6.0 g, 32.258 mmol). LCMS: Method B, 3.422 min, MS: ES+ 204.4 (M+18).

To a stirred solution of methyl 5-methyl-4-nitroisoxazole-3-carboxylate (compound 25.4; 1.0, 5.374 mmol) in methanol (10 mL) was added Raney nickel (1.0 mL) at ambient temperature and the reaction mixture was stirred under H₂ atmosphere for 2 h. The resulting reaction mixture was filtered through celite and the filtrate was concentrated under reduced pressure yielding methyl 4-amino-5-methylisoxazole-3-carboxylate (compound 25.3; 0.8 g, 5.128 mmol). LCMS: Method B, 1.859 min, MS: ES+ 157.3 (M+1)

To a stirred solution of 4-bromo phenyl boronic acid (CAS Number 19788-35-3, 1.29 g, 6.408 mmol) in dichloromethane (15 mL) were added Cu(OAc)₂ (0.87 g, 4.806 mmol) and TEA (0.7 mL, 4.806 mmol) at ambient temperature and the reaction mixture was stirred for 5 minutes. Methyl 4-amino-5-methylisoxazole-3-carboxylate (compound 25.3; 0.5 g, 3.204 mmol) was added to the reaction mixture at ambient temperature and stirred for 1 hour along with O₂ gas purging. The resulting reaction mixture was poured into water (100 mL) and extracted with dichloromethane (2×50 mL). The combined organic phase was dried over Na₂SO₄, filtered and concentrated under reduced pressure. The resulting crude material was purified by flash chromatography (8.5% EtOAc in hexane) yielding methyl 4-((4-bromophenyl)amino)-5-methylisoxazole-3-carboxylate (compound 25.2; 0.45 g, 1.452 mmol). LCMS: Method B, 4.614 min, MS: ES+ 328.3 (M+18).

To a stirred solution of methyl 4-((4-bromophenyl)amino)-5-methylisoxazole-3-carboxylate (compound 25.2; 0.2 g, 0.645 mmol) in 1, 4-dioxane:water (2:1, 12 mL) were added Na₂CO₃ (0.2 g, 1.935 mmol) and 4-cyano phenyl boronic acid (CAS No. 126747-14-6; 0.3 g, 1.935 mmol) at ambient temperature and the reaction mixture was purged with N₂ gas for 15 minutes. PdCl₂(dppf) (0.05 g, 0.064 mmol) was added to the reaction mixture at ambient temperature and heating was applied. The reaction mixture was stirred at 80° C. for 1 hour. The resulting reaction mixture was cooled to ambient temperature and poured into water (50 mL). The resulting reaction mixture was extracted with EtOAc (2×20 mL). The combined organic phase was dried over Na₂SO₄, filtered and concentrated under reduced pressure. The resulting crude material was purified by flash chromatography (10.5% EtOAc in hexane) yielding methyl 4-((4′-cyano-[1,1′-biphenyl]-4-yl)amino)-5-methylisoxazole-3-carboxylate (compound 25.1; 0.09 g, 0.270 mmol). LCMS: Method B, 4.550 min, MS: ES+334.4 (M+1).

To a stirred solution of methyl 4-((4′-cyano-[1,1′-biphenyl]-4-yl)amino)-5-methylisoxazole-3-carboxylate (compound 25.1; 0.08 g, 0.240 mmol) in THF:water (2:1, 6 mL) was added LiOH.H₂O (0.02 g, 0.360 mmol) at ambient temperature and the reaction mixture was stirred for 1 hour. The resulting reaction mixture was poured into saturated NaHCO₃ solution (20 mL) and washed with EtOAc (2×10 mL). The obtained aqueous layer was acidified (pH=4) with 1N HCl and extracted with EtOAc (2×10 mL). The combined organic phase was dried over Na₂SO₄, filtered and concentrated under reduced pressure. The resulting solid was triturated with n-pentane (2×3 mL) and dried yielding 4-((4′-cyano-[1,1′-biphenyl]-4-yl)amino)-5-methylisoxazole-3-carboxylic acid (compound 25; 0.03 g, 0.094 mmol). LCMS: Method B, 4.001 min, MS: ES+ 320.3 (M+1); ¹H NMR (400 MHz, MeOD) δ ppm: 7.71-7.76 (m, 4H), 7.54 (m, 2H), 6.75 (dd, J=2.0, 6.8 Hz, 2H), 2.39 (s, 3H).

### Example 26. 4-((4-Bromophenyl)amino)-5-methylisoxazole-3-carboxylic Acid (26)

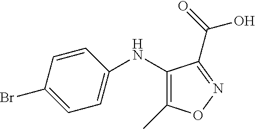 [SMILES: Cc1onc(C(=O)O)c1Nc1ccc(Br)cc1]

The title compound was synthesized by hydrolysis of compound 25.2 according to Scheme 8, step e. LCMS: Method B, 3.742 min, MS: ES+ 314.3 (M+18); ¹H NMR (400 MHz, MeOD) δ ppm: 7.26 (d, J=8.0 Hz, 2H), 6.56 (d, J=8.0 Hz, 2H), 2.36 (s, 3H).

### Example 27. 4-(3-Bromophenylamino)-5-methylisoxazole-3-carboxylic Acid (27)

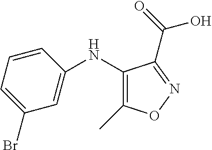 [SMILES: Cc1onc(C(=O)O)c1Nc1cccc(Br)c1]

The title compound was synthesized via steps a, b, c₁, and e of Scheme 8, following the procedures described in Example 25 using 3-bromophenylboronic acid (CAS Number 89598-96-9) in step c₁ instead of 4-bromophenyl boronic acid. LCMS: Method B, 3.985 min, MS: ES+ 297.2 (M+1); ¹H NMR (400 MHz, MeOD) δ ppm: 7.05 (t, J=8.0 Hz, 1H), 6.86 (dd, J=1.2, 8.0 Hz, 1H), 6.77 (t, J=2.0 Hz, 1H), 6.57 (dd, J=1.6, 8.0 Hz, 1H), 2.36 (s, 3H).

### Example 28. 4-((4′-Cyano-[1,1′-biphenyl]-3-yl)amino)-5-methylisoxazole-3-carboxylic Acid (28)

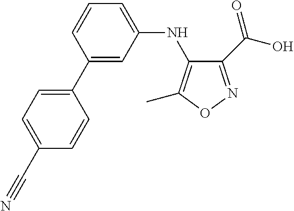 [SMILES: Cc1onc(C(=O)O)c1Nc1cccc(-c2ccc(C#N)cc2)c1]

The title compound was synthesized via steps a, b, c₁, d, and e of Scheme 8, following the procedures described in Example 25 using 3-bromophenylboronic acid (CAS Number 89598-96-9) in step C₁ instead of 4-bromophenyl boronic acid. LCMS: Method B, 4.323 min, MS: ES+ 320.4 (M+1); ¹H NMR (400 MHz, DMSO-d₆) δ ppm: 14.00 (s, 1H), 11.03 (s, 1H), 7.91 (d, J=8.8 Hz, 2H), 7.75 (d, J=8.4 Hz, 2H), 7.25 (d, J=8.0 Hz, 1H), 6.57 (d, J=8.4 Hz, 1H), 6.86 (t, J=1.6 Hz, 1H), 6.66 (dd, J=1.6, 8.0 Hz, 1H), 2.33 (s, 3H).

### Example 29. 4-((4-Fluorobenzyl)amino)-5-methylisoxazole-3-carboxylic Acid (29)

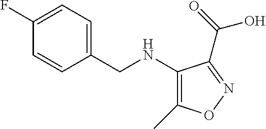 [SMILES: Cc1onc(C(=O)O)c1NCc1ccc(F)cc1]

To a stirred solution of methyl 4-amino-5-methylisoxazole-3-carboxylate (compound 25.3; 0.25 g, 1.602 mmol) in acetonitrile (5.0 mL) were added K₂CO₃ (0.44 g, 3.205 mmol) and 4-fluoro benzyl bromide (CAS No. 459-46-1) (0.3 g, 1.602 mmol) at ambient temperature and the reaction mixture was stirred for 2 hours. The resulting reaction mixture was poured into water (50 mL) and extracted with EtOAc (2×25 mL). The combined organic phase was dried over Na₂SO₄, filtered and concentrated under reduced pressure. The resulting crude material was purified by flash chromatography (12.8% EtOAc in hexane) yielding methyl 4-((4-fluorobenzyl)amino)-5-methylisoxazole-3-carboxylate, compound 29.1. LCMS: Method B, 4.315 min, MS: ES+ 265.3 (M+1)

The title compound 29 was synthesized by hydrolysis of methyl 4-((4-fluorobenzyl)amino)-5-methylisoxazole-3-carboxylate, compound 29.1, following the procedure described in Example 25. LCMS: Method B, 3.447 min, MS: ES+ 251.4 (M+1); ¹H NMR (400 MHz, DMSO-d₆) δ ppm: 9.27 (br s, 1H), 7.31-7.35 (q, J=5.6, 8.4 Hz, 2H), 7.14 (t, J=9.2 Hz, 2H), 4.22 (s, 2H), 2.30 (s, 3H).

### Example 30. 4-((4-Cyanophenyl)amino)-5-methylisoxazole-3-carboxylic Acid (30)

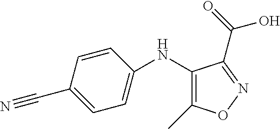 [SMILES: Cc1onc(C(=O)O)c1Nc1ccc(C#N)cc1]

To a stirred solution of methyl 4-amino-5-methylisoxazole-3-carboxylate (compound 25.3; 0.2 g, 1.281 mmol) in 1, 4-dioxane (10 mL) were added K₂CO₃ (0.35 g, 2.563 mmol) and 4-bromobenzonitrile (CAS No. 623-00-7) (0.23 g, 1.281 mmol) at ambient temperature. The reaction mixture was purged with N₂ gas at ambient temperature for 15 minutes. Pd₂dba₃ (0.06 g, 0.064 mmol) and Xantphos (0.07 g, 0.128 mmol) were added to the reaction mixture at ambient temperature and heating was applied. The reaction mixture was stirred at 100° C. for 2 hours. The resulting reaction mixture was cooled to ambient temperature and poured into water (50 mL). The resulting reaction mixture was extracted with EtOAc (2×25 mL). The combined organic phase was dried over Na₂SO₄, filtered and concentrated under reduced pressure. The resulting crude material was purified by flash chromatography (25% EtOAc in hexane) yielding methyl 4-((4-cyanophenyl)amino)-5-methylisoxazole-3-carboxylate (compound 30.1; 0.08 g, 0.311 mmol). LCMS: Method B, 3.876 min, MS: ES+ 258.4 (M+1).

The title compound 30 was synthesized by hydrolysis of methyl 4-((4-cyanophenyl)amino)-5-methylisoxazole-3-carboxylate, compound 30.1, following the procedure described in Example 25. LCMS: Method B, 3.242 min, MS: ES+ 244.3 (M+1); ¹H NMR (400 MHz, DMSO-d₆) δ ppm: 13.93 (br s, 1H), 8.17 (s, 1H), 7.52 (d, J=8.8 Hz, 2H), 6.65 (d, J=8.4 Hz, 2H), 2.32 (s, 3H).

### Example 31. 4-((4-(4-Cyanophenoxy)benzyl)amino)-5-methylisoxazole-3-carboxylic Acid (31)

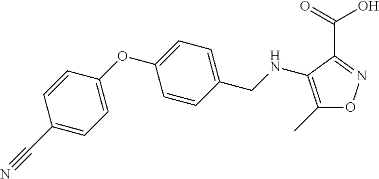 [SMILES: Cc1onc(C(=O)O)c1NCc1ccc(Oc2ccc(C#N)cc2)cc1]

The title compound 31 was synthesized via steps a, b, c₂, and e of Scheme 8, following the procedure described in Example 29 using 4-(4-(bromomethyl)phenoxy)benzonitrile (CAS Number 321337-61-5) in step C₂ instead of 4-fluoro benzyl bromide. Compound 32 was also formed, which was isolated and characterized as described below. LCMS: Method B, 4.051 min, MS: ES+ 350.5 (M+1); ¹H NMR (400 MHz, DMSO-d₆) δ ppm: 7.82-7.86 (m, 2H), 7.39 (d, J=8.4 Hz, 2H), 7.11 (d, J=8.4 Hz, 2H), 7.05-7.09 (m, 2H), 4.29 (s, 2H), 2.34 (s, 3H).

### Example 32. 4-(bis(4-(4-Cyanophenoxy)benzyl)amino)-5-methylisoxazole-3-carboxylic Acid (32)

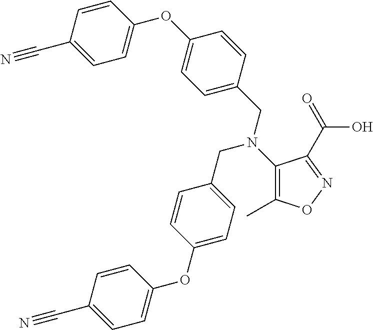 [SMILES: Cc1onc(C(=O)O)c1N(Cc1ccc(Oc2ccc(C#N)cc2)cc1)Cc1ccc(Oc2ccc(C#N)cc2)cc1]

LCMS: Method B, 4.655 min, MS: ES+ 557.5 (M+1); ¹H NMR (400 MHz, DMSO-d₆) δ ppm: 14.10 (br s, 1H), 7.81-7.84 (m, 4H), 7.35 (d, J=8.4 Hz, 4H), 7.08 (d, J=8.4 Hz, 4H), 7.03-7.06 (m, 4H), 4.17 (s, 4H), 1.97 (s, 3H).

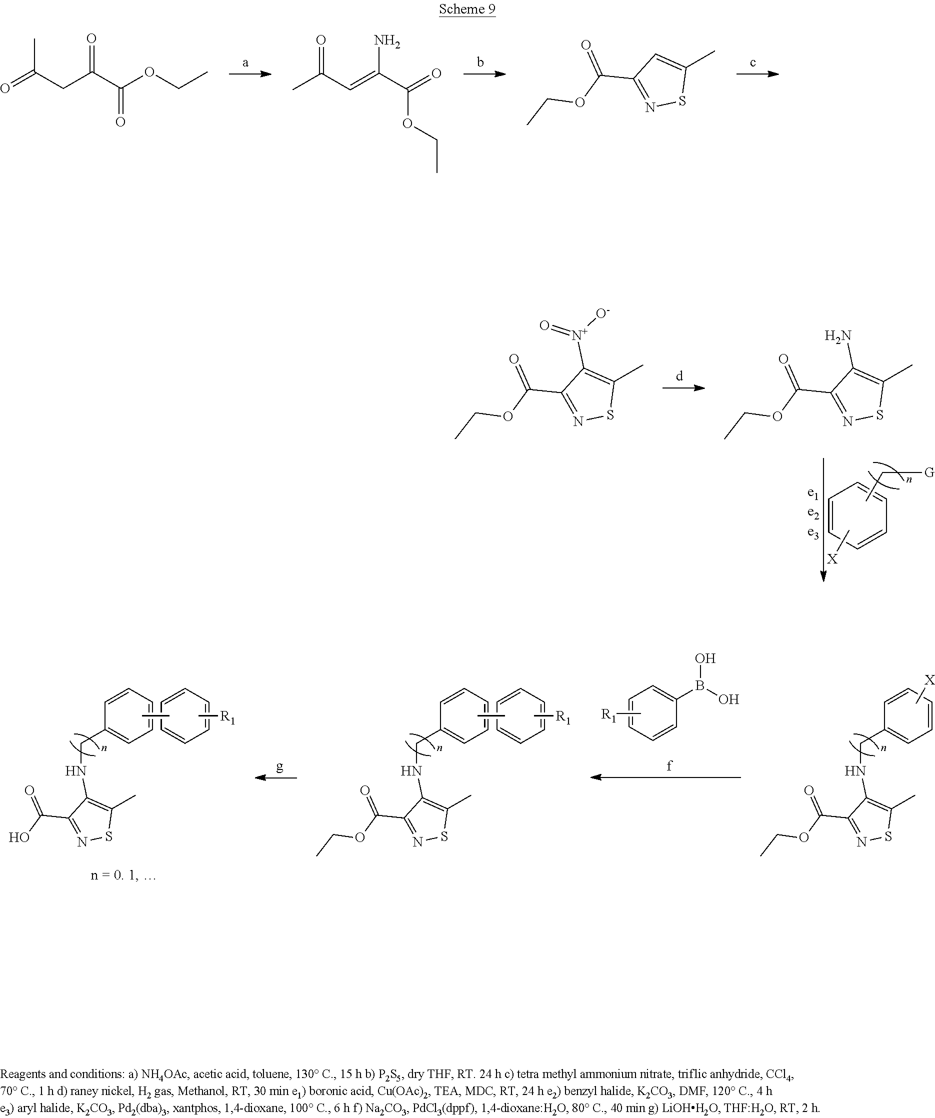

### Example 33. 4-((4′-Cyano-[1,1′-biphenyl]-4-yl)amino)-5-methylisothiazole-3-carboxylic Acid (33)

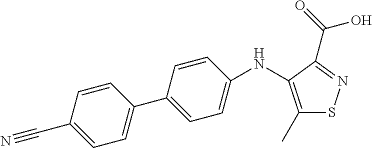 [SMILES: Cc1snc(C(=O)O)c1Nc1ccc(-c2ccc(C#N)cc2)cc1]

The title compound was prepared according to Scheme 9. To a suspension of tetra methyl ammonium nitrate (2.4 g, 17.536 mmol) in CCl₄ (10 mL) was added triflic anhydride (1.7 g, 17.536 mmol) drop wise at 0° C. under nitrogen atmosphere. The resulting suspension was stirred at ambient temperature for 90 minutes. Ethyl 5-methylisothiazole-3-carboxylate (Intermediate C, Example 13; 1.0 g, 5.845 mmol) was added to the reaction mixture at ambient temperature. The reaction mixture was heated at 70° C. for 1 h. The resulting reaction mixture was cooled to ambient temperature and poured into saturated NaHCO₃ solution (50 mL). The resulting reaction mixture was extracted with dichloromethane (2×50 mL). The combined organic phase was dried over Na₂SO₄, filtered and concentrated under reduced pressure yielding ethyl 5-methyl-4-nitroisothiazole-3-carboxylate (compound 33.4; 0.8 g, 3.703 mmol). LCMS: Method B, 3.927 min, MS: ES+ 217.4 (M+1).

To a solution of ethyl 5-methyl-4-nitroisothiazole-3-carboxylate (compound 33.4; 0.75 g, 3.471 mmol) in methanol (20 mL) was added Raney nickel (1 mL) at ambient temperature and the reaction mixture was stirred under H₂ atmosphere for 30 minutes. The reaction mixture was filtered through celite and the resulting filtrate was concentrated under reduced pressure yielding ethyl 4-amino-5-methylisothiazole-3-carboxylate (compound 33.3; 0.41 g, 2.204 mmol). LCMS: Method A, 1.582 min, MS: ES+ 187.16 (M+1).

To a stirred solution of 4-bromophenylboronic acid (CAS Number 19788-35-3, 2.1 g, 10.481 mmol) in dichloromethane (30 mL) were added Cu(OAc)₂ (2.22 g, 12.227 mmol) and TEA (1.7 mL, 12.227 mmol) at ambient temperature and the reaction mixture was stirred for 5 minutes. Ethyl 4-amino-5-methylisothiazole-3-carboxylate (compound 33.3; 0.65 g, 3.493 mmol) was added to the reaction mixture at ambient temperature and stirred for 24 hours along with O₂ gas purging. The resulting reaction mixture was poured into water (100 mL) and extracted with MDC (2×50 mL). The combined organic phase was dried over Na₂SO₄, filtered and concentrated under reduced pressure. The resulting crude material was purified by flash chromatography (10% EtOAc in hexane) yielding ethyl 4-((4-bromophenyl)amino)-5-methylisothiazole-3-carboxylate (compound 33.2; 0.62 g, 1.823 mmol). LCMS: Method A, 2.622 min, MS: ES+ 340.68 (M+1).

To a stirred solution of ethyl 4-((4-bromophenyl)amino)-5-methylisothiazole-3-carboxylate (compound 33.2; 0.25 g, 0.735 mmol) in 1, 4-dioxane:water (4:1, 10 mL) were added Na₂CO₃ (0.23 g, 2.205 mmol) and 4-cyano phenyl boronic acid (CAS No. 126747-14-6) (0.32 g, 0.2.205 mmol) at ambient temperature. The reaction mixture was purged with N₂ gas at ambient temperature for 15 minutes. PdCl₂(dppf) (0.05 g, 0.073 mmol) was added to the reaction mixture at ambient temperature and heating was applied. The reaction mixture was stirred at 80° C. for 40 minutes. The resulting reaction mixture was cooled to ambient temperature and poured into water (50 mL). The resulting reaction mixture was extracted with EtOAc (2×30 mL). The combined organic phase was dried over Na₂SO₄, filtered and concentrated under reduced pressure. The resulting crude material was purified by flash chromatography (7% EtOAc in hexane) yielding ethyl 4-((4′-cyano-[1,1′-biphenyl]-4-yl)amino)-5-methylisothiazole-3-carboxylate (compound 33.1; 0.09 g, 0.247 mmol). LCMS: Method A, 2.677 min, MS: ES+ 363.94 (M+1).

To a stirred solution of ethyl 4-((4′-cyano-[1,1′-biphenyl]-4-yl)amino)-5-methylisothiazole-3-carboxylate (compound 33.1; 0.045 g, 0.124 mmol) in THF:water (8:2, 5 mL) was added LiOH.H₂O (0.005 g, 0.123 mmol) at ambient temperature and the reaction mixture was stirred for 2 hours. The reaction mixture was poured into saturated NaHCO₃ solution (10 mL) and washed with EtOAc (2×15 mL). The aqueous layer was then acidified (pH=4) with 1N HCl and extracted with EtOAc (2×20 mL). The combined organic phase was dried over Na₂SO₄, filtered and concentrated under reduced pressure. The resulting solid was triturated with n-pentane:diethyl ether (1:1, 6 mL) and dried yielding 4-((4′-cyano-[1,1′-biphenyl]-4-yl)amino)-5-methylisothiazole-3-carboxylic acid (compound 33; 0.012 g, 0.035 mmol). LCMS: Method A, 2.304 min, MS: ES− 233.9 (M−1); ¹H NMR (400 MHz, DMSO-d6) δ ppm: 7.75-7.89 (m, 4H), 7.51-7.61 (m, 2H), 6.64-6.78 (m, 2H), 2.32 (s, 3H).

### Example 34. 4-((4-Bromophenyl)amino)-5-methylisothiazole-3-carboxylic Acid (34)

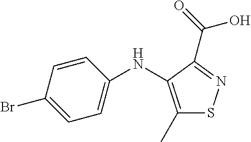 [SMILES: Cc1snc(C(=O)O)c1Nc1ccc(Br)cc1]

The title compound was synthesized by hydrolysis of compound 33.2 using the procedure described in Example 33. LCMS: Method A, 2.192 min, MS: ES− 310.7 (M−1); ¹H NMR (400 MHz, MeOD) δ ppm: 7.31 (d, J=8.8 Hz, 2H), 6.63 (d, J=8.8 Hz, 2H), 2.32 (s, 3H).

### Example 35. 4-((3-Bromophenyl)amino)-5-methylisothiazole-3-carboxylic Acid (35)

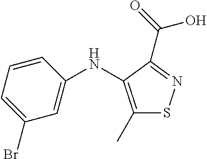 [SMILES: Cc1snc(C(=O)O)c1Nc1cccc(Br)c1]

The title compound was synthesized via steps a, b, c, d, and e₁ of Scheme 9, following the procedure described in Example 33 using 3-bromophenylboronic acid (CAS Number 89598-96-9) in step e₁ instead of 4-bromophenylboronic acid. LCMS: Method D, 9.936 min, MS: ES+ 313.4 (M+1); ¹H NMR (400 MHz, MeOD) δ ppm: 7.09 (t, J=8.0 Hz, 1H), 6.93 (d, J=8.0 Hz, 1H), 6.81 (s, 1H), 6.62 (d, J=8.0 Hz, 1H), 2.35 (s, 3H).

### Example 36. 4-((4′-Cyano-[1,1′-biphenyl]-3-yl)amino)-5-methylisothiazole-3-carboxylic Acid (36)

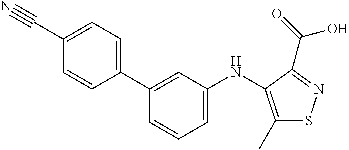 [SMILES: Cc1snc(C(=O)O)c1Nc1cccc(-c2ccc(CN)cc2)c1]

The title compound was synthesized via steps a, b, c, d, e₁, and f of Scheme 9, following the procedure described in Example 33 using 3-bromophenylboronic acid (CAS Number 89598-96-9) in step e₁ instead of 4-bromophenylboronic acid. LCMS: Method A, 2.300 min, MS: ES− 333.72 (M−1); ¹H NMR (400 MHz, MeOD) δ ppm: 7.75-7.80 (m, 4H), 7.34 (t, J=8.0 Hz, 1H), 7.14 (d, J=7.6 Hz, 1H), 7.00 (s, 1H), 6.75 (d, J=8.0 Hz, 1H), 2.36 (s, 3H).

### Example 37. 4-((4-Fluorobenzyl)amino)-5-methylisothiazole-3-carboxylic Acid (37)

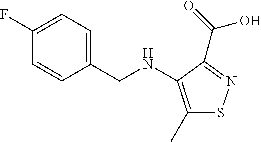 [SMILES: Cc1snc(C(=O)O)c1NCc1ccc(F)cc1]

To a stirred solution of ethyl 4-amino-5-methylisothiazole-3-carboxylate (compound 33.3; 0.11 g, 0.591 mmol) in DMF (4 mL) were added K₂CO₃ (0.24 g, 1.773 mmol) and 4-fluoro benzyl bromide (CAS No. 459-46-1, 0.18 g, 0.945 mmol) at ambient temperature. The reaction mixture was stirred at 120° C. for 4 h. The resulting reaction mixture was cooled to ambient temperature, poured into water (30 mL) and extracted with EtOAc (2×20 mL). The combined organic phase was dried over Na₂SO₄, filtered and concentrated under reduced pressure. The resulting crude material was purified by flash chromatography (13% EtOAc in hexane) yielding ethyl 4-((4-fluorobenzyl)amino)-5-methylisothiazole-3-carboxylate (compound 37.1; 0.095 g, 0.323 mmol). LCMS: Method A, 2.470 min, MS: ES+ 294.86 (M+1).

The title compound 37 was synthesized by hydrolysis of ethyl 4-((4-fluorobenzyl)amino)-5-methylisothiazole-3-carboxylate, compound 37.1, following the procedure descried in Example 33. LCMS: Method A, 1.706 min, MS: ES+ 266.8 (M+1); ¹H NMR (400 MHz, DMSO-d₆) δ ppm: 7.32-7.35 (t, J=8.4 Hz, 2H), 7.14 (t, J=8.8 Hz, 2H), 4.38 (s, 2H), 2.40 (s, 3H).

### Example 38. 4-((4-Cyanophenyl)amino)-5-methylisothiazole-3-carboxylic Acid (38)

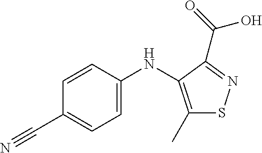 [SMILES: Cc1snc(C(=O)O)c1Nc1ccc(C#N)cc1]

To a stirred solution of ethyl 4-amino-5-methylisothiazole-3-carboxylate (compound 33.3; 0.35 g, g, 1.880 mmol) in 1, 4-dioxane (6 mL) were added K₂CO₃ (0.52 g, 3.760 mmol) and 4-bromobenzonitrile (CAS No. 623-00-7, 0.34 g, 1.880 mmol) at ambient temperature. The reaction mixture was purged with N₂ gas at ambient temperature for 15 minutes. Pd₂(dba)₃ (0.09 g, 0.094 mmol) and xantphos (0.11 g, 0.188 mmol) were added to the reaction mixture at ambient temperature and heating was applied. The reaction mixture was stirred at 100° C. for 6 hours. The resulting reaction mixture was poured into water (75 mL) and extracted with EtOAc (2×50 mL). The combined organic phase was dried over Na₂SO₄, filtered and concentrated under reduced pressure. The resulting residue was purified by flash chromatography (17% EtOAc in hexane) yielding ethyl 4-((4-cyanophenyl)amino)-5-methylisothiazole-3-carboxylate (compound 38.1; 0.39 g, 1.376 mmol). LCMS: Method B, 4.044 min, MS: ES+ 288.3 (M+1).

The title compound 38 was synthesized by hydrolysis of ethyl 4-((4-cyanophenyl)amino)-5-methylisothiazole-3-carboxylate, compound 38.1, following the procedure described in Example 33. LCMS: Method A, 1.869 min, MS: ES− 257.6 (M−1); ¹H NMR (400 MHz, DMSO-d₆) δ ppm: 13.40 (br s, 1H), 8.47 (br s, 1H), 7.52 (d, J=8.8 Hz, 2H), 6.61 (d, J=8.8 Hz, 2H), 2.31 (s, 3H).

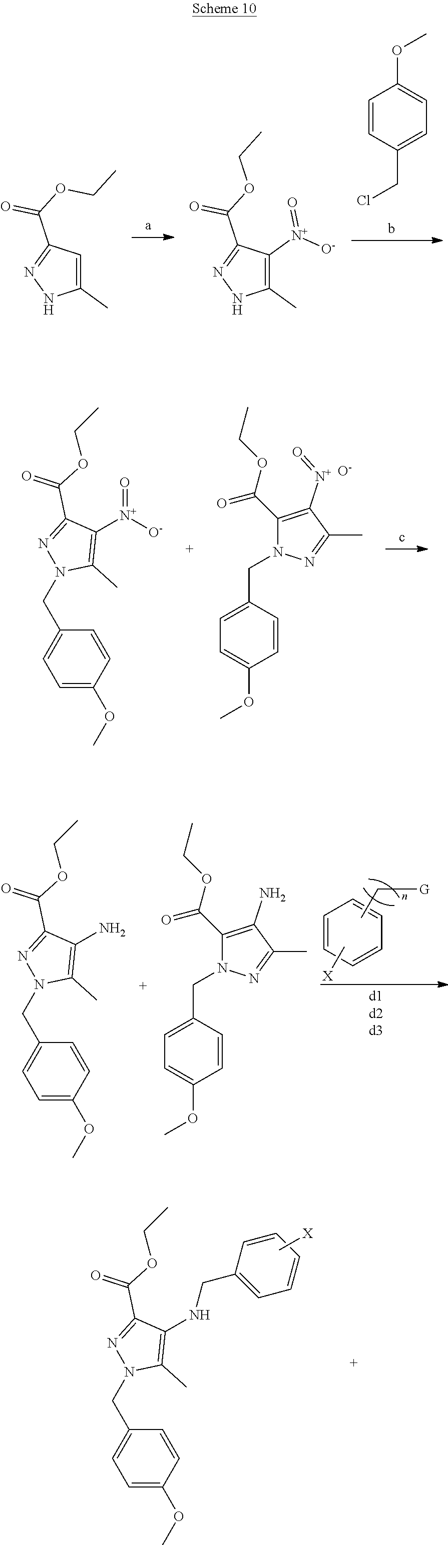

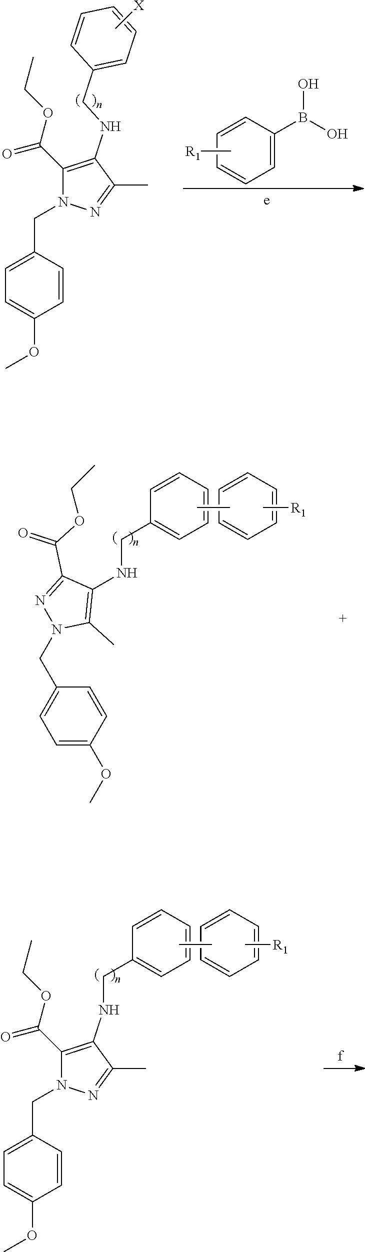

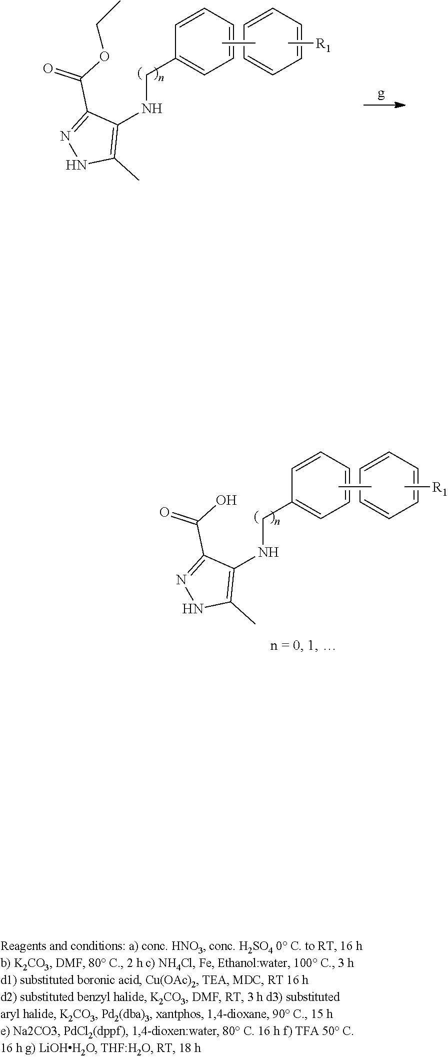

### Example 39. 4-((4′-Cyano-[1,1′-biphenyl]-4-yl)amino)-5-methyl-H-pyrazole-3-carboxylic Acid (39)

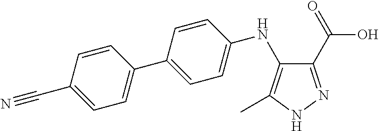 [SMILES: Cc1[nH]nc(C(=O)O)c1Nc1ccc(-c2ccc(C#N)cc2)cc1]

The title compound was prepared according to Scheme 10. A mixture of conc. HNO₃ (4.5 mL) and conc. H₂SO₄ (17.1 mL) was prepared at 0° to 5° C. and stirred for 15 minutes. Ethyl 3-methylpyrazole-5-carboxylate (CAS No: 4027-57-0) (3 g, 19.455 mmol) was added to the nitration mixture at 0° to 5° C. and the obtained reaction mixture was allowed to reach ambient temperature. The reaction mixture was stirred at ambient temperature for 16 h and poured into cold water (300 mL). The obtained mixture was basified by slow addition of saturated NaHCO₃ and extracted with EtOAc (2×500 mL). The combined organic phase was dried over Na₂SO₄, filtered and concentrated under reduced pressure yielding ethyl 5-methyl-4-nitro-1H-pyrazole-3-carboxylate (compound 39.6; 1.9 g, 9.539 mmol). LCMS: Method A, 1.780 min, MS: ES− 198.1 (M−1).

To a solution of ethyl 5-methyl-4-nitro-1H-pyrazole-3-carboxylate (compound 39.6; 1.0 g, 5.020 mmol) in DMF (15 mL) were added K₂CO₃ (2.635 g, 19.066 mmol) and 4-methoxy benzylchloride (0.522 g, 3.333 mmol) at ambient temperature. The reaction mixture was heated at 80° C. for 2 h and then allowed to cool to ambient temperature. In parallel another reaction at 0.9 g scale was carried out by following same procedure. Upon completion both the resulting reaction mixtures were mixed together and taken up for further workup and isolation procedures.

The resulting mixture was poured into water (100 mL) and extracted with EtOAc (2×70 mL). The combined organic phase was dried over Na₂SO₄, filtered and concentrated under reduced pressure. The resulting residue was purified by column chromatography (18% EtOAc in hexane) yielding ethyl 1-(4-methoxybenzyl)-3-methyl-4-nitro-1H-pyrazole-5-carboxylate (compound 39.5a; 0.5 g, 1.566 mmol) LCMS: Method B, 4.777 min, MS: ES+320.4 (M+1); ¹H NMR (400 MHz, MeOD) δ ppm: 7.20-7.24 (m, 2H), 6.88-6.92 (m 2H), 5.33 (s, 2H), 4.38 (q, J=7.2, 14.4 Hz, 2H), 3.80 (s, 3H), 2.47 (s, 3H), 1.30 (t, J=7.2 Hz, 3H). and (25% EtOAc in hexane) ethyl 1-(4-methoxybenzyl)-5-methyl-4-nitro-1H-pyrazole-3-carboxylate (compound 39.5b; 1.1 g, 3.446 mmol) LCMS: Method B, 4.492 min, MS: ES+337.4 (M+18); ¹H NMR (400 MHz, MeOD) δ ppm: 7.19-7.22 (m, 2H), 6.91-6.94 (m, 2H), 5.36 (s, 2H), 4.41 (q, J=6.8, 14 Hz, 2H), 3.79 (s, 3H), 2.56 (s, 3H), 1.38 (t, J=7.2 Hz, 3H).

To a stirred solution of ethyl 1-(4-methoxybenzyl)-5-methyl-4-nitro-1H-pyrazole-3-carboxylate (compound 39.5b; 1.1 g, 3.446 mmol) in ethanol:water (3:1, 20 mL) were added Fe powder (0.962 g, 17.232 mmol) and NH₄Cl (3.68 g, 68.928 mmol) at ambient temperature. The reaction mixture was heated at 100° C. for 3 h. The resulting reaction mixture was allowed to cool to ambient temperature and filtered through celite bed. The filtrate was poured in water (50 mL) and extracted with EtOAc (2×30 mL). The combined organic phase was dried over Na₂SO₄, filtered and concentrated under reduced pressure yielding ethyl 4-amino-1-(4-methoxybenzyl)-5-methyl-1H-pyrazole-3-carboxylate (compound 39.4; 0.9 g, 3.112 mmol). LCMS: Method A, 1.850 min, MS: ES+ 290.0 (M+1); ¹H NMR (400 MHz, DMSO-d₆) δ ppm: 7.06 (d, J=8.4 Hz, 2H), 6.90 (d, J=8.8 Hz, 2H), 5.19 (s, 2H), 4.48 (s, 2H), 4.24 (q, J=7.2, 14.0 Hz, 2H), 3.72 (s, 3H), 2.05 (s, 3H), 1.28 (t, J=7.2 Hz, 3H).

To a stirred solution of 4-bromophenylboronic acid (CAS Number 19788-35-3) (1.88 g, 9.402 mmol) in dichloromethane (15 mL) were added Cu(OAc)₂ (0.850 g, 4.701 mmol) and TEA (0.6 mL, 4.701 mmol) at ambient temperature and the reaction mixture was stirred for 5 minutes. Ethyl 4-amino-1-(4-methoxybenzyl)-5-methyl-1H-pyrazole-3-carboxylate (compound 39.4; 0.9 g, 3.112 mmol) was added in to the reaction mixture at ambient temperature and stirred for 24 h along with O₂ gas purging. The resulting reaction mixture was poured into water (80 mL) and extracted with dichloromethane (3×25 mL). The combined organic phase was dried over Na₂SO₄, filtered and concentrated under reduced pressure. The resulting crude material was purified by flash chromatography (20% EtOAc in hexane) yielding ethyl 4-((4-bromophenyl)amino)-1-(4-methoxybenzyl)-5-methyl-1H-pyrazole-3-carboxylate (compound 39.3; 1.28 g, 2.888 mmol). LCMS: Method A, 2.776 min, MS: ES+ 445.8 (M+2).

To a stirred solution of ethyl 4-((4-bromophenyl)amino)-1-(4-methoxybenzyl)-5-methyl-1H-pyrazole-3-carboxylate (compound 39.3; 0.5 g, 1.128 mmol) in 1, 4-dioxane:water (2:1, 10 mL) were added Na₂CO₃ (0.358 g, 3.337 mmol) and 4-cyano phenyl boronic acid (CAS No. 126747-14-6; 0.3497 g, 3.383 mmol) at ambient temperature and the reaction mixture was purged with N₂ gas for 15 minutes. PdCl₂(dppf) (0.082 g, 0.112 mmol) was added to the reaction mixture at ambient temperature and heating was applied. The reaction mixture was stirred at 80° C. for 16 hour. The resulting reaction mixture was cooled to ambient temperature and poured into water (60 mL). The resulting reaction mixture was extracted with EtOAc (3×25 mL). The combined organic phase was dried over Na₂SO₄, filtered and concentrated under reduced pressure. The resulting crude material was purified by column chromatography (25% EtOAc in hexane) yielding ethyl 4-((4′-cyano-[1,1′-biphenyl]-4-yl)amino)-1-(4-methoxybenzyl)-5-methyl-1H-pyrazole-3-carboxylate (compound 39.2; 0.360 g, 0.772 mmol). LCMS: Method A, 2.804 min, MS: ES+ 467 (M+1).

A solution of ethyl 4-((4′-cyano-[1,1′-biphenyl]-4-yl)amino)-1-(4-methoxybenzyl)-5-methyl-1H-pyrazole-3-carboxylate (compound 39.2; 0.3 g, 0.643 mmol) was prepared in TFA (4 mL) at ambient temperature. The reaction mixture was heated at 50° C. for 16 hours. The resulting reaction mixture was poured in to cold water (80 mL) and neutralized by saturated NaHCO₃. The resulting reaction mixture was extracted with EtOAc (2×30 mL). The combined organic phase was dried over Na₂SO₄, filtered and concentrated under reduced pressure. The resulting crude material was purified by column chromatography (35% EtOAc in hexane) yielding ethyl 4-((4′-cyano-[1,1′-biphenyl]-4-yl)amino)-5-methyl-1H-pyrazole-3-carboxylate (compound 39.1; 0.15 g, 0.433 mmol). LCMS: Method A, 2.392 min, MS: ES+347 (M+1).

To a stirred solution of ethyl 4-((4′-cyano-[1,1′-biphenyl]-4-yl)amino)-5-methyl-1H-pyrazole-3-carboxylate (compound 39.1; 0.15 g, 0.433 mmol) in THF:water (1:1, 6 mL) was added LiOH.H₂O (0.090 g, 2.144 mmol) at ambient temperature. The resulting reaction mixture was stirred at 60° for 16 h. The reaction mixture was poured in water (80 mL) and washed with EtOAc (3×30 mL). The obtained aqueous layer was acidified (pH=4) with 1N HCl and extracted with EtOAc (3×25 mL). The combined organic phase was dried over Na₂SO₄, filtered and concentrated under reduced pressure. The resulting solid was triturated with n-pentane (10 mL) and dried. The resulting crude was purified by prep HPLC (using acetonitrile/water/0.1% HCOOH) yielding 4-((4′-cyano-[1,1′-biphenyl]-4-yl)amino)-5-methyl-1H-pyrazole-3-carboxylic acid (compound 39; 0.007 g, 0.022 mmol). LCMS: Method B, 3.984 min, MS: ES+ 319.4; ¹H NMR (400 MHz, DMSO-d₆) δ ppm: 12.50 (br s, 1H), 7.76-7.82 (m, 4H), 7.55 (d, J=8.4 Hz, 2H), 6.70 (d, J=8.4 Hz, 2H), 2.02 (s, 3H).

### Example 40. 4-((4-Bromophenyl)amino)-5-methyl-1H-pyrazole-3-carboxylic Acid (40)

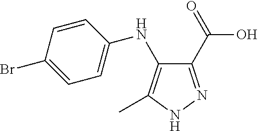 [SMILES: Cc1[nH]nc(C(=O)O)c1Nc1ccc(Br)cc1]

The title compound was synthesized by hydrolysis of compound 39.3, following the procedure described in Example 39 using LiOH.H₂O instead of NaOH. LCMS: Method A, 2.010 min, MS: ES+ 295.9 (M+1); ¹H NMR (400 MHz, DMSO-d₆) δ ppm: 13.16 (br s, 1H), 7.28 (s, 1H), 7.21 (d, J=8.4 Hz, 2H), 6.48 (d, J=8.8 Hz, 2H), 2.01 (s, 3H).

### Example 41. 4-((3-Bromophenyl)amino)-5-methyl-1H-pyrazole-3-carboxylic Acid (41)

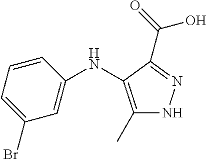 [SMILES: Cc1[nH]nc(C(=O)O)c1Nc1cccc(Br)c1]

The title compound was synthesized via steps a, b, c, d₁, and g of Scheme 10, following the procedure described in Example 39 using 3-bromophenylboronic acid (CAS Number 89598-96-9) in step d₁ instead of 4-bromophenyl boronic acid. LCMS: Method A, 1.917 min, MS: ES+ 295.82 (M+1); ¹H NMR (400 MHz, MeOD) δ ppm: 7.03 (t, J=8.0 Hz 1H), 6.81-6.84 (m, 1H), 6.74 (t, J=2.0 Hz, 1H), 6.57 (dd, J=1.6, 8.4 Hz, 1H), 2.14 (s, 3H).

### Example 42. 4-((4′-Cyano-[1,1′-biphenyl]-3-yl)amino)-5-methyl-1H-pyrazole-3-carboxylic Acid (42)

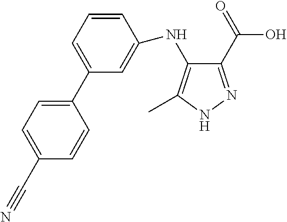 [SMILES: Cc1[nH]nc(C(=O)O)c1Nc1cccc(-c2ccc(C#N)cc2)c1]

The title compound was synthesized via steps a, b, c, d₁, e, f, and g of Scheme 10, following the procedure described in Example 39 using 3-bromophenylboronic acid (CAS Number 89598-96-9) in step d₁ instead of 4-bromophenyl boronic acid. LCMS: Method B, 4.046 min, MS: ES+ 319.4 (M+1); ¹H NMR (400 MHz, DMSO-d₆) δ ppm: 13.14 (br s, 1H), 7.89 (d, J=8.8 Hz, 2H), 7.73 (d, J=8.4 Hz, 2H), 7.23 (t, 8.0 Hz, 1H), 6.98 (d, J=7.4 Hz, 1H), 6.85 (s, 1H), 6.60-6.64 (m, 1H), 2.06 (s, 3H)

### Example 43. 4-((4-Fluorobenzyl)amino)-3-methyl-1H-pyrazole-5-carboxylic Acid (43)

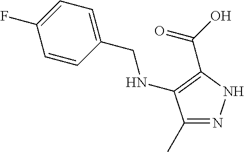 [SMILES: Cc1n[nH]c(C(=O)O)c1NCc1ccc(F)cc1]

To a stirred solution of ethyl 1-(4-methoxybenzyl)-3-methyl-4-nitro-1H-pyrazole-5-carboxylate (compound 39.5a; 2.2 g, 6.892 mmol) in ethanol:water (3:1, 25 mL) were added Fe powder (1.9 g, 34.025 mmol) and NH₄Cl (7.4 g, 138.322 mmol) at ambient temperature. The reaction mixture was heated at 100° C. for 16 hours. The resulting reaction mixture was allowed to cool to ambient temperature and filtered through celite bed. The filtrate was poured in water (100 mL) and extracted with EtOAc (3×50 mL). The combined organic phase was dried over Na₂SO₄, filtered and concentrated under reduced pressure yielding ethyl 4-amino-1-(4-methoxybenzyl)-3-methyl-1H-pyrazole-5-carboxylate (compound 43.3; 1.9 g, 6.571 mmol). LCMS: Method A, 2.115 min, MS: ES+ 289.96 (M+1); ¹H NMR (400 MHz, DMSO-d₆) δ ppm: 7.01-7.04 (m, 2H), 6.83-6.86 (m, 2H), 5.36 (s, 2H), 4.84 (s, 2H), 4.24 (q, J=6.8, 14.0 Hz, 2H), 3.70 (s, 3H), 2.07 (s, 3H), 1.25 (t, J=6.8 Hz, 3H).

To a stirred solution of ethyl 4-amino-1-(4-methoxybenzyl)-3-methyl-1H-pyrazole-5-carboxylate (compound 43.3; 0.3 g, 1.037 mmol) in acetonitrile (4.0 mL) were added K₂CO₃ (0.29 g, 2.170 mmol) and 4-fluoro benzyl bromide (CAS No. 459-46-1) (0.19 g, 1.037 mmol) at ambient temperature and the reaction mixture was stirred for 3 hours. In parallel another reaction at 0.1 g scale was carried out by following same procedure. Upon completion both the resulting reaction mixtures were mixed together and taken up for further workup and isolation procedures.

The resulting mixture was poured into water (50 mL) and extracted with EtOAc (2×25 mL). The combined organic phase was dried over Na₂SO₄, filtered and concentrated under reduced pressure. yielding a mixture of ethyl 4-((4-fluorobenzyl)amino)-1-(4-methoxybenzyl)-3-methyl-TH-pyrazole-5-carboxylate and ethyl 4-(bis(4-fluorobenzyl)amino)-1-(4-methoxybenzyl)-3-methyl-TH-pyrazole-5-carboxylate (compounds 43.2a and 43.2b, respectively; 0.171 g). LCMS: Method B, 4.879 min, MS: ES+ 398.46 (M+1), 5.205 min, MS: ES+ 506.51 (M+1). The obtained mixture was directly used in the next step.

A solution of ethyl 4-((4-fluorobenzyl)amino)-1-(4-methoxybenzyl)-3-methyl-H-pyrazole-5-carboxylate and ethyl 4-(bis(4-fluorobenzyl)amino)-1-(4-methoxybenzyl)-3-methyl-1H-pyrazole-5-carboxylate (compounds 43.2a and 43.2b; 0.150 g) was prepared in TFA (4 mL) at ambient temperature and then heated at 50° C. for 2 hours. The resulting reaction mixture was allowed to cool to ambient temperature poured in to cold water (80 mL). The resulting mixture was basified by slow addition of saturated NaHCO₃ solution and extracted with EtOAc (2×30 mL). The combined organic phase was dried over Na₂SO₄, filtered and concentrated under reduced pressure. The resulting crude material was purified by flash chromatography yielding (15% EtOAc in hexane) ethyl 4-(bis(4-fluorobenzyl)amino)-3-methyl-1H-pyrazole-5-carboxylate (compound 43.1b; 0.069 g, 0.248 mmol) and (23% EtOAc in hexane) ethyl 4-((4-fluorobenzyl)amino)-3-methyl-H-pyrazole-5-carboxylate (compound 43.1a; 0.138 g, 0.358 mmol) LCMS: Method B, 4.353 min, MS: ES+ 278.44 (M+1)

The title compound 43 was synthesized by hydrolysis of ethyl 4-((4-fluorobenzyl)amino)-3-methyl-1H-pyrazole-5-carboxylate, compound 43.1a, following the procedure described in Example 39. LCMS: Method B, 3.513 min, MS: ES+ 250.39 (M+1); ¹H NMR (400 MHz, DMSO-d₆) δ ppm: 12.48 (br s, 1H), 7.29-7.33 (m, 2H), 7.12 (t, J=8.8 Hz, 2H), 4.27 (s, 1H), 2.14 (s, 3H).

### Example 44. 4-(bis(4-Fluorobenzyl)amino)-3-methyl-1H-pyrazole-5-carboxylic Acid (44)

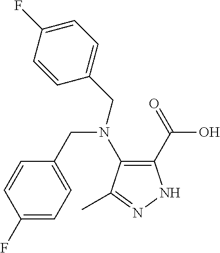 [SMILES: Cc1n[nH]c(C(=O)O)c1N(Cc1ccc(F)cc1)Cc1ccc(F)cc1]

Title compound 44 was synthesized by hydrolysis of ethyl 4-(bis(4-fluorobenzyl)amino)-3-methyl-1H-pyrazole-5-carboxylate, compound 43.1b, following the procedure described in Example 39. LCMS: Method B, 4.811 min, MS: ES+ 358.40 (M+1); ¹H NMR (400 MHz, DMSO-d₆) δ ppm: 12.80 (br s, 1H), 7.23-7.26 (m, 4H), 7.08 (t, J=8.8 Hz, 4H), 4.11 (s, 4H), 1.76 (s, 3H).

### Example 45. 4-((4-Cyanophenyl)amino)-5-methyl-1H-pyrazole-3-carboxylic Acid (45)

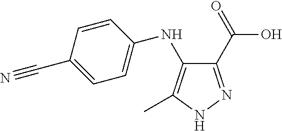 [SMILES: Cc1[nH]nc(C(=O)O)c1Nc1ccc(C#N)cc1]

To a stirred solution of ethyl 4-amino-1-(4-methoxybenzyl)-3-methyl-1H-pyrazole-5-carboxylate (compound 43.3; 0.25 g, 0.864 mmol) in 1, 4-dioxane (10 mL) were added K₂CO₃ (0.238 g, 1.722 mmol) and 4-bromobenzonitrile (CAS No. 623-00-7) (0.157 g, 0.862 mmol) at ambient temperature. The reaction mixture was purged with N₂ gas at ambient temperature for 15 minutes. Pd₂dba₃ (0.039 g, 0.042 mmol) and Xantphos (0.049 g, 0.084 mmol) were added to the reaction mixture at ambient temperature and heating was applied. The reaction mixture was stirred at 90° C. for 15 hours. The resulting reaction mixture was cooled to ambient temperature and poured into water (80 mL). The resulting reaction mixture was extracted with EtOAc (2×40 mL). The combined organic phase was dried over Na₂SO₄, filtered and concentrated under reduced pressure. The resulting crude material was purified by flash chromatography (15% EtOAc in hexane) yielding ethyl 4-((4-cyanophenyl)amino)-1-(4-methoxybenzyl)-3-methyl-1H-pyrazole-5-carboxylate (compound 45.1; 0.1 g, 0.256 mmol). LCMS: Method A, 2.727 min, MS: ES+ 390.9 (M+1).

The title compound 45 was synthesized via steps f and g of Scheme 10, following the procedure described in Example 39 using ethyl 4-((4-cyanophenyl)amino)-1-(4-methoxybenzyl)-3-methyl-H-pyrazole-5-carboxylate, compound 45.1, in step f instead of ethyl 4-((4′-cyano-[1,1′-biphenyl]-4-yl)amino)-1-(4-methoxybenzyl)-5-methyl-H-pyrazole-3-carboxylate. LCMS: Method A, 1.588 min, MS: ES− 240.8 (M−1); ¹H NMR (400 MHz, DMSO-d₆) δ ppm: 13.12 (br s, 1H), 8.091 (s, 1H) 7.46 (d, J=8.8 Hz, 2H), 6.58 (d, J=8.4 Hz, 2H), 2.03 (s, 3H).

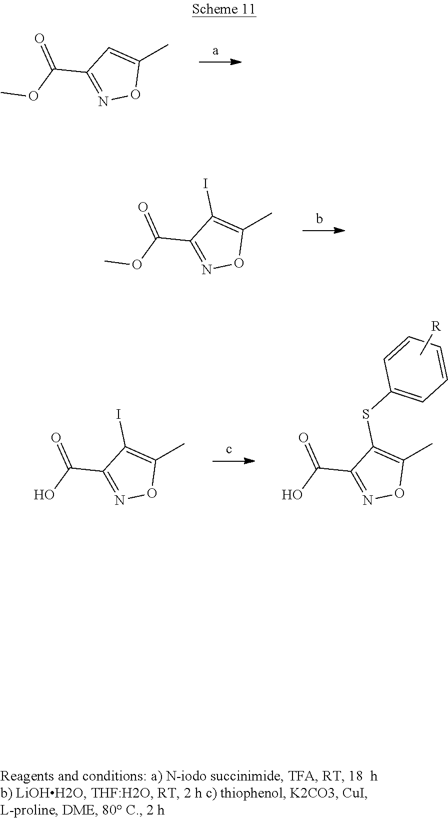

### Example 46. 4-((4-Fluorophenyl)thio)-5-methylisoxazole-3-carboxylic Acid (46)

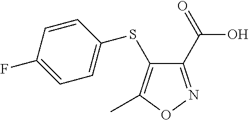 [SMILES: Cc1onc(C(=O)O)c1Sc1ccc(F)cc1]

The title compound was prepared according to Scheme 11. To a stirred solution of methyl 5-methylisoxazole-3-carboxylate (CAS Number 19788-35-3) (1.0 g, 7.087 mmol) in TFA (10 mL) was added N-iodo succinimide (2.4 g, 10.630 mmol) at ambient temperature and the reaction mixture was stirred for 18 hours. The resulting reaction mixture was poured into saturated NaHCO₃ (100 mL) and extracted with EtOAc (2×50 mL). The combined organic layer was washed with saturated sodium thiosulphate solution (100 mL), dried over Na₂SO₄, filtered and concentrated under reduced pressure yielding methyl 4-iodo-5-methylisoxazole-3-carboxylate (compound 46.2; 1.89 g, 7.078 mmol). LCMS: Method A, 2.063 min, MS: ES+ 267.7 (M+1).

To a stirred solution of methyl 4-iodo-5-methylisoxazole-3-carboxylate (compound 46.2; 0.5 g, 1.872 mmol) in THF:water (2:1, 10 mL) was added LiOH.H₂O (0.12 g, 2.808 mmol) at ambient temperature and the reaction mixture was stirred at ambient temperature for 2 hours. The resulting reaction mixture was poured into saturated NaHCO₃ solution (50 mL) and washed with EtOAc (2×20 mL). The obtained aqueous layer was acidified (pH=4) with 1N HCl and extracted with EtOAc (2×20 mL). The combined organic phase was dried over Na₂SO₄, filtered and concentrated under reduced pressure yielding 4-iodo-5-methylisoxazole-3-carboxylic acid (compound 46.1; 0.4 g, 1.581 mmol). LCMS: Method B, 1.001 min, MS: ES+ 254.2 (M+1).

To a stirred solution of 4-iodo-5-methylisoxazole-3-carboxylic acid (compound 46.1; 0.25 g, 0.988 mmol) in 1,2-dimethoxyethane (10 mL) were added K₂CO₃ (0.3 g, 1.976 mmol) and 4-fluorothiophenol (CAS No. 371-42-6, 0.1 g, 0.988 mmol) at ambient temperature. The reaction mixture was purged with N₂ gas at ambient temperature for 15 minutes. CuI (0.02 g, 0.0988 mmol) and L-proline (0.02 g, 0.1976 mmol) were added to the reaction mixture at ambient temperature and heating was applied. The reaction mixture was stirred at 80° C. for 2 hours. The resulting reaction mixture was cooled to ambient temperature and poured into saturated NaHCO₃ solution (20 mL) and washed with EtOAc (2×30 mL). The obtained aqueous layer was acidified (pH=4) with 1N HCl and extracted with EtOAc (2×20 mL). The combined organic phase was dried over Na₂SO₄, filtered and concentrated under reduced pressure. The resulting crude material was purified by flash chromatography (100% Chloroform) yielding 4-((4-fluorophenyl)thio)-5-methylisoxazole-3-carboxylic acid (compound 46; 0.05 g, 0.197 mmol). LCMS: Method A, 2.185 min, MS: ES− 208.1 (M-COOH); ¹H NMR (400 MHz, DMSO-d₆, 80° C.) δ ppm: 11.81 (br s, 1H), 7.30-7.35 (m, 2H), 7.17-7.22 (m, 2H), 2.33 (s, 1H).

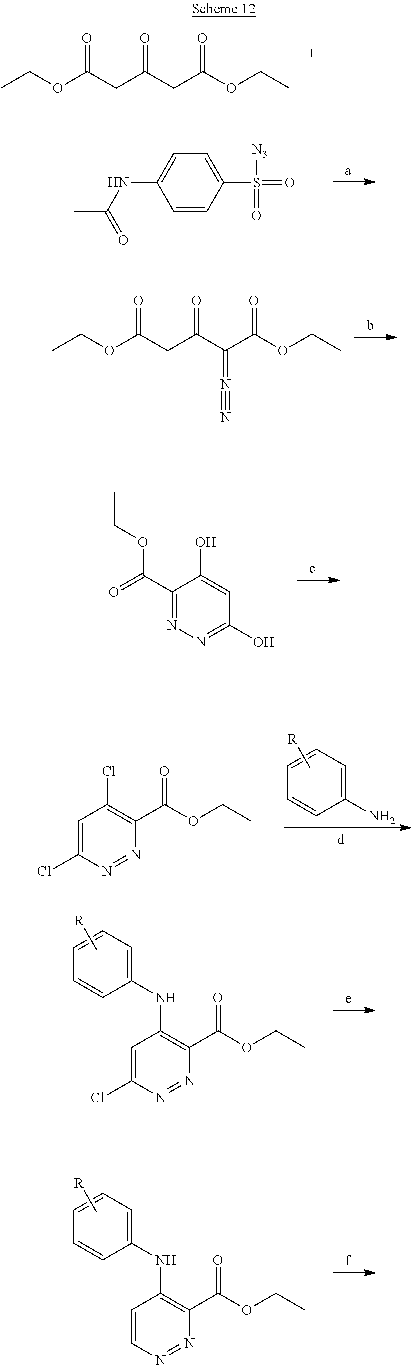

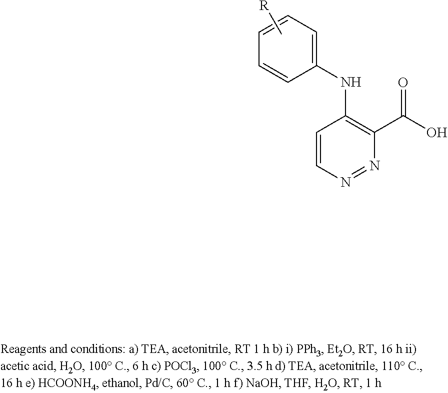

### Example 47. 4-((4-Fluorophenyl)amino)pyridazine-3-carboxylic Acid (47)

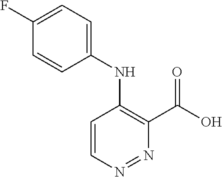 [SMILES: O=C(O)c1nnccc1Nc1ccc(F)cc1]

The title compound was prepared according to Scheme 12. To a stirred solution of diethyl 1,3-acetonedicarboxylate (CAS Number 1830-54-2, 8.2 mL, 49.455 mmol) and triethylamine (7.5 mL, 53.906 mmol) in acetonitrile (220 mL) was added 4-acetamidobenzenesulfonylazide (CAS Number 2158-14-7, 12.11 g, 50.439 mmol) at 0° C. in small portions. The reaction was stirred at ambient temperature for 1 h. The resulting solid was filtered under vacuum and washed with hexane:diethyl ether (1:1, 250 ml). The resulting filtrate was concentrated and suspended in hexane:diethyl ether (1:1, 100 mL). The resulting suspension was shirred for 30 minutes at ambient temperature and filtered off under vacuum. The filtrate was concentrated under vacuum yielding diethyl 2-diazo-3-oxopentanedioate (compound 47.5; 11.1 g, 48.669 mmol). LCMS: Method B, 3.888 min, MS: ES+ 228.07 (M+1).

To a stirred solution of diethyl 2-diazo-3-oxopentanedioate (compound 47.5; 11.0 g, 51.353 mmol) in diethyl ether (90 mL) was added triphenylphosphine (14.1 g, 53.921 mmol) at ambient temperature and the reaction mixture was stirred for 16 h. The resulting reaction mixture was concentrated under vacuum and the obtained residue was dissolved in acetic acid:water (9:1, 100 mL). The obtained reaction mixture was heated at 100° C. for 6 h. The resulting reaction mixture was cooled to ambient temperature and concentrated under vacuum. The obtained residue was taken up in 1N NaOH solution (50 mL) and washed with EtOAc (3×500 mL). The obtained aqueous layer was acidified (pH=3) with 50% citric acid solution and extracted with EtOAc (3×500 mL). The combined organic phase was dried over Na₂SO₄, filtered and concentrated under reduced pressure yielding ethyl 4, 6-dihydroxypyridazine-3-carboxylate (compound 47.4; 3.2 g, 17.387 mmol). Method B, 0.611 min, MS: ES+ 184.04 (M+1).

A solution of ethyl 4, 6-dihydroxypyridazine-3-carboxylate (compound 47.4; 3.2 g, 17.387 mmol) in phosphorus oxychloride (35 mL, 226.037 mmol) was heated at 100° C. for 3.5 h. The resulting reaction mixture was cooled to ambient temperature and the excess of phosphorus oxychloride was removed under vacuum. The traces phosphorus oxychloride was further removed by azeotropic distillation with chloroform (50 mL). The resulting residue was taken up in to ice water and extracted with EtOAc (3×500 mL). The combined organic layers were washed with water (100 mL), brine (100 mL), dried over Na₂SO₄, filtered and concentrated under reduced pressure. The resulting crude material was purified by flash chromatography (60-70% EtOAc in hexane) yielding ethyl 4, 6-dichloropyridazine-3-carboxylate (compound 47.3; 2.5 g, 11.310 mmol). LCMS: Method B, 3.750 min, MS: ES+219.98 (M+1).

To a stirred solution of ethyl 4, 6-dichloropyridazine-3-carboxylate (compound 47.3; 0.5 g, 2.273 mmol) in acetonitrile (5.0 mL) were added 4-fluoro aniline (CAS Number 371-40-4, 0.37 g, 3.410 mmol) and triethylamine (0.95 mL, 6.821 mmol) at ambient temperature and the reaction mixture was heated at 100° C. for 16 h. The resulting reaction mixture was cooled to ambient temperature and poured in to saturated NaHCO₃ solution (15 mL). The resulting reaction mixture was extracted with EtOAc (3×25 mL). The combined organic phase was dried over Na₂SO₄, filtered and concentrated under reduced pressure yielding ethyl 6-chloro-4-((4-fluorophenyl)amino)pyridazine-3-carboxylate (compound 47.2; 0.465 g, 1.576 mmol). LCMS: Method B, 4.446 min, MS: ES+ 295.05 (M+1).

To a stirred solution of ethyl 6-chloro-4-((4-fluorophenyl)amino)pyridazine-3-carboxylate (compound 47.2; 0.25 g, 0.847 mmol) in ethanol (2.0 mL) were added 10% dry Pd/C (0.02 g) and ammonium formate (0.06 g, 0.932 mmol) at ambient temperature and the reaction mixture was heated at 60° C. for 1 h. The resulting reaction mixture was carefully filtered through celite and the filtrate was concentrated under reduced pressure. The obtained mixture was poured in to water (10 mL) and neutralized by solid NaHCO₃. The resulting mixture was extracted with EtOAc (3×20 mL). The combined organic phase was dried over Na₂SO₄, filtered and concentrated under reduced pressure yielding ethyl 4-((4-fluorophenyl)amino)pyridazine-3-carboxylate (compound 47.1; 0.146 g, 0.559 mmol). LCMS: Method B, 4.220 min, MS: ES+ 261.09 (M+1).

To a stirred solution of ethyl 4-((4-fluorophenyl)amino)pyridazine-3-carboxylate (compound 47.1; 0.06 g, 0.229 mmol) in THF:water (1:1, 4.0 mL) was added NaOH (0.009 g, 0.229 mmol) at ambient temperature. The reaction mixture was stirred for 1 h. post which THF was removed under vacuum and the obtained aqueous layer was washed with EtOAc (3×25 mL) to remove undesired impurities. The obtained aqueous layer was acidified (pH=6.5) with 1N HCl and the resulting precipitates were collected by filtration under vacuum, washed with water (5.0 mL) followed by n-pentene (10 mL). The resulting solid was dried under vacuum yielding 4-((4-fluorophenyl)amino)pyridazine-3-carboxylic acid (compound 47; 0.017 g, 0.072 mmol). LCMS: Method B, 3.232 min, MS: ES+ 234.4 (M+1); ¹H NMR (400 MHz, DMSO-d₆+a drop of triethylamine) δ ppm: 12.108 (s, 1H), 8.57 (d, J=6.0 Hz, 1H), 7.27-7.31 (m, 2H), 7.20-7.25 (m, 2H), 7.02 (d, J=6.4 Hz, 1H).

### Example 48. 4-((4′-Cyano-[1,1′-biphenyl]-3-yl)amino)pyridazine-3-carboxylic Acid (48)

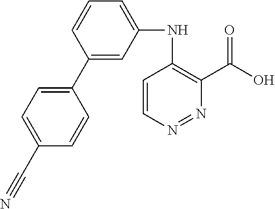 [SMILES: N#Cc1ccc(-c2cccc(Nc3ccnnc3C(=O)O)c2)cc1]

The title compound was synthesized via step a, b, c, d, e and f of Scheme 12, the procedure described in Example 47 using 3′-amino-[1,1′-biphenyl]-4-carbonitrile (CAS No: 149505-72-6) in step d instead of 4-fluoro aniline.

Compound 48, sodium salt: LCMS: Method B, 3.940 min, MS: ES+ 317.4 (M+1); ¹H NMR (400 MHz, MeOD) δ ppm: 8.60 (d, J=6.4 Hz, 1H), 7.82-7.88 (m, 4H), 7.54-7.62 (m, 3H), 7.39-7.42 (m, 1H), 7.32 (d, J=6.4 Hz, 1H).

Compound 48, free acid: LCMS: Method A, 1.618 min, MS: ES+ 316.9 (M+1); ¹H NMR (400 MHz, DMSO-d₆+a drop of triethylamine) δ ppm: 12.433 (s, 1H), 8.60 (d, J=6.0 Hz, 1H), 7.92-7.98 (m, 4H), 7.47-7.57 (m, 3H), 7.31-7.36 (m, 1H), 7.27 (d, J=6.4 Hz, 1H).

### Example 49. 4-((2,4-Dimethoxybenzyl)amino)pyridazine-3-carboxylic Acid Na Salt (49)

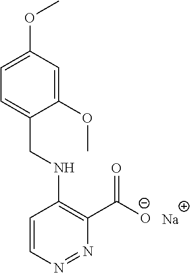 [SMILES: COc1ccc(CNc2ccnnc2C(=O)[O-])c(OC)c1 ; [Na+]]

The title compound was synthesized via steps a, b, c, d, e, and f of Scheme 12, following the procedure described in Example 47 using 2,4-dimethoxybenzylamine (CAS No: 20781-20-8) in step d instead of 4-fluoro aniline. LCMS: Method B, 3.613 min, MS: ES+ 290.38 (M+1); ¹H NMR (400 MHz, D₂O) δ ppm: 8.32 (d, J=6.8 Hz, 1H), 7.08 (d, J=8.4 Hz, 1H), 6.65 (d, J=6.4 Hz, 1H), 6.51 (d, J=2.4 Hz, 1H), 6.41 (dd, J=2.4, 8.4 Hz, 1H), 4.22 (s, 2H), 3.74 (s, 3H), 3.67 (s 3H).

### Example 50. 4-((4-Fluorobenzyl)amino)pyridazine-3-carboxylic Acid, sodium Salt (50)

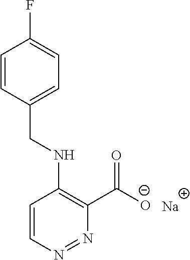 [SMILES: O=C([O-])c1nnccc1NCc1ccc(F)cc1 ; [Na+]]

The title compound was synthesized via step a, b, c, d, e, and f of Scheme 12, following the procedure described in Example 47 using 4-fluorobenzylamine (CAS No: 140-75-0) in step d instead of 4-fluoro aniline. LCMS: Method B, 3.356 min, MS: ES+ 248.5 (M+1); ¹H NMR (400 MHz, DMSO-d₆) δ ppm: 10.31 (t, J=6.0 Hz, 1H), 8.45 (d, J=6.0 Hz, 1H), 7.35-7.38 (m, 2H), 7.15-7.20 (m, 2H), 6.58 (d, J=6.0 Hz, 1H), 4.39 (d, J=6.0 Hz, 2H).

### Example 51. 4-(Benzylthio)-5-methylisoxazole-3-carboxylic Acid (51)

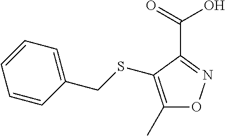 [SMILES: Cc1onc(C(=O)O)c1SCc1ccccc1]

The title compound was prepared according to Scheme 13. To a solution of ethyl 2,4-dioxopentanoate (CAS Number 615-79-2, 2.0 g, 12.650 mmol) in dichloromethane was drop wise added sulfuryl chloride (2.22 g, 16.445 mmol) at ambient temperature under nitrogen atmosphere. The resulting solution was stirred at ambient temperature for 3 h. The resulting reaction mixture was concentrated under reduced pressure yielding ethyl 3-chloro-2,4-dioxopentanoate (compound 51.3; 2.2 g, 11.457 mmol). LCMS: Method B, 1.185 min, MS: ES+ 193.4 (M+1).

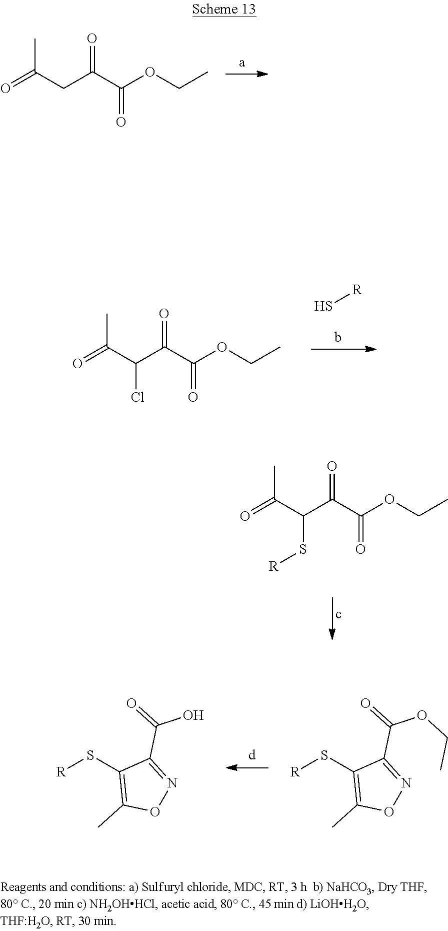

To a stirred solution of ethyl 3-chloro-2,4-dioxopentanoate (compound 51.3; 0.5 g, 2.603 mmol) in dry THF (10 mL) were added NaHCO₃ (0.656 g, 7.811 mmol) and phenylmethanethiol (0.32 g, 2.603 mmol) at ambient temperature. The reaction mixture was heated at 80° C. for 20 min. The resulting reaction mixture was cooled to ambient temperature, poured into water (100 mL) and extracted with EtOAc (2×50 mL). The combined organic phase was dried over Na₂SO₄, filtered and concentrated under reduced pressure. The resulting crude material was purified by flash chromatography (17% EtOAc in hexane) yielding ethyl 3-(benzylthio)-2,4-dioxopentanoate (compound 51.2; 0.61 g, 2.177 mmol). LCMS: Method B, 3.737 min, MS: ES+ 281.4 (M+1).

To a stirred solution of ethyl 3-(benzylthio)-2,4-dioxopentanoate (compound 51.2; 0.2 g, 0.714 mmol) in acetic acid (3 mL) was added NH₂OH.HCl (0.496 g, 7.140 mmol) at ambient temperature. The reaction mixture was heated at 80° C. for 45 min. The resulting reaction mixture was cooled to ambient temperature, poured into water (50 mL) and extracted with EtOAc (2×30 mL). The combined organic phase was dried over Na₂SO₄, filtered and concentrated under reduced pressure. The resulting crude material was purified by flash chromatography (20% EtOAc in hexane) yielding ethyl 4-(benzylthio)-5-methylisoxazole-3-carboxylate (compound 51.1; 0.11 g, 0.397 mmol). LCMS: Method B, 4.690 min, MS: ES+278.5 (M+1).

To a stirred solution of ethyl 4-(benzylthio)-5-methylisoxazole-3-carboxylate (compound 51.1; 0.1 g, 0.360 mmol) in THF:water (2:0.5, 2.5 mL) was added LiOH.H₂O (0.015 g, 0.360 mmol) at ambient temperature. The resulting reaction mixture was stirred at ambient temperature for 30 min. The reaction mixture was poured into saturated NaHCO₃ solution (10 mL) and washed with EtOAc (2×15 mL). The aqueous layer was then acidified (pH=4) with dilute HCl and extracted with EtOAc (2×20 mL). The combined organic phase was dried over Na₂SO₄, filtered and concentrated under reduced pressure. The resulting solid was triturated with n-pentane (10 mL) and dried yielding 4-(benzylthio)-5-methylisoxazole-3-carboxylic acid (compound 51; 0.065 g, 0.261 mmol). LCMS: Method B, 3.743 min, MS: ES+ 250.4 (M+1); ¹H NMR (400 MHz, DMSO-d6) δ ppm: 14.73 (br s, 1H), 7.20-7.31 (m, 3H), 7.11-7.18 (m, 2H), 4.07 (s, 2H), 1.93 (s, 3H).

### Example 52. 5-((3-(pyridin-2-yl)phenyl)amino)-1,2,3-thiadiazole-4-carboxylic Acid Na Salt (52)

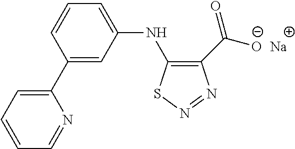 [SMILES: O=C([O-])c1nnsc1Nc1cccc(-c2ccccn2)c1 ; [Na+]]

The title compound was synthesized via steps a, b & c of Scheme 7, following similar synthetic procedures as mentioned for Example 7A, using 3-(pyridin-2-yl)aniline (CAS Number 15889-32-4) in step a instead of 4-(1H-pyrazol-1-yl)aniline. LCMS: Method B, 3.101 min, MS: ES+ 299.4 (M+1); ¹H NMR (400 MHz, DMSO-d₆) δ ppm: 13.52 (s, 1H), 8.58-8.73 (m, 2H), 7.85-8.20 (m, 4H), 7.55-7.71 (m, 1H), 7.36-7.44 (m, 1H).

### Example 53. 4-((4-(4-cyanophenoxy)phenyl)amino)-5-methylisoxazole-3-carboxylic Acid (53)

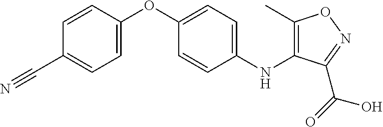 [SMILES: Cc1onc(C(=O)O)c1Nc1ccc(Oc2ccc(C#N)cc2)cc1]

The title compound was synthesized via steps a, b, c₁ & e of Scheme 8, following similar synthetic procedures as mentioned for Example 25, using (4-(4-cyanophenoxy)-phenyl)boronic acid (CAS Number 947162-05-2) in step c₁ instead of 4-bromo phenyl boronic acid. LCMS: Method B, 4.095 min, MS: ES+ 336.4 (M+1); ¹H NMR (400 MHz, DMSO-d₆) δ ppm: 13.86 (br s, 1H), 7.77-7.81 (m, 2H), 7.37 (br s, 1H), 6.98-7.02 (m, 2H), 6.92-6.95 (m, 2H), 6.63-6.67 (m, 2H), 2.33 (s, 3H).

### Example 54. 4-((4-(4-fluorobenzyl)phenyl)amino)-5-methylisoxazole-3-carboxylic Acid (54)

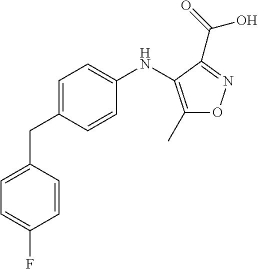 [SMILES: Cc1onc(C(=O)O)c1Nc1ccc(Cc2ccc(F)cc2)cc1]

The title compound was synthesized via steps a, b, c₁ & e of Scheme 8, following similar synthetic procedures as mentioned for Example 25, using (4-(4-fluorobenzyl)phenyl)-boronic acid (synthesis described below) in step c₁ instead of 4-bromo phenyl boronic acid. LCMS: Method B, 4.347 min, MS: ES+ 327.5 (M+1); ¹H NMR (400 MHz, DMSO-d₆) δ ppm: 13.78 (br s, 1H), 7.20-7.23 (m, 2H), 7.06-7.10 (m, 2H), 6.96 (d, J=8.4 Hz, 2H), 6.49 (d, J=8.4 Hz, 2H), 3.78 (s, 1H), 2.28 (s, 3H).

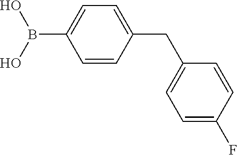 [SMILES: OB(O)c1ccc(Cc2ccc(F)cc2)cc1]

(4-(4-Fluorobenzyl)phenyl)boronic acid. Step a: To a suspension of 4-bromobenzaldehyde (CAS Number 1122-91-4) (2.5 g, 13.511 mmol) in 1,4-dioxane (60 mL) was added 4-methylbenzenesulfonohydrazide (2.52 g, 13.511 mmol) at ambient temperature. The resulting suspension was heated at 90° C. for 3 h. (4-fluorophenyl)boronic Acid (2.835 g, 20.266 mmol) and K₂CO₃ (2.797 g, 20.266 mmol) were added in the reaction mixture at ambient temperature. The reaction mixture was heated at 90° C. for 3 h. The resulting reaction mixture was poured into water (100 mL) and extracted with EtOAc (2×60 mL). The combined organic phase was dried over Na₂SO₄, filtered and concentrated under reduced pressure. The resulting crude material was purified by flash chromatography (10% EtOAc in hexane) yielding 1-bromo-4-(4-fluorobenzyl)benzene (1.85 g, 7.008 mmol). ¹H NMR (400 MHz, DMSO-d₆) δ ppm: 7.14-7.44 (m, 2H), 7.10-7.15 (m, 2H), 7.04-7.07 (m, 2H), 6.96-7.02 (m, 2H), 3.92 (s, 2H).

Step b: To a stirred solution of 1-bromo-4-(4-fluorobenzyl)benzene (1.0 g, 3.788 mmol) in THF (20 mL) was added n-BuLi (1.6 M in Hexane, 4.4 mL, 7.576 mmol) at −78° C. The resulting reaction mixture was stirred for 5 min, triisopropylborate (2.85 g, 15.152 mmol) was added at −78° C. The resulting reaction mixture was gradually warmed to the ambient temperature and stirred for 30 min. The resulting reaction mixture was poured into 1 N NH₄Cl solution (60 mL) and extracted with EtOAc (2×50 mL). The combined organic phase was dried over Na₂SO₄, filtered and concentrated under reduced pressure (4-(4-cyanophenoxy)phenyl)boronic acid (0.4 g, 1.673 mmol).

### Example 55. 4-((4-((4-fluorophenyl)ethynyl)phenyl)amino)-5-methylisoxazole-3-carboxylic Acid (55)

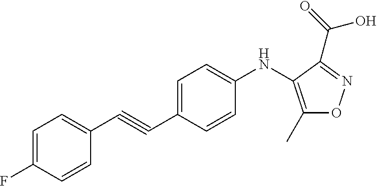 [SMILES: Cc1onc(C(=O)O)c1Nc1ccc(C#Cc2ccc(F)cc2)cc1]

The title compound was synthesized via steps a, b, c₃ & e of Scheme 8, following similar synthetic procedures as mentioned for Example 30, using 1-bromo-4-((4-fluorophenyl)ethynyl)benzene (CAS Number 64583-19-3) in step C₃ instead of 4-bromobenzonitrile. LCMS: Method B, 4.423 min, MS: ES+ 337.4 (M+1); ¹H NMR (400 MHz, DMSO-d₆) δ ppm: 7.48-7.52 (m, 2H), 7.13-7.28 (m, 4H), 6.52 (d, J=8.0 Hz, 2H), 1.82 (s, 3H).

### Example 56. 4-((4-(4-fluorophenethyl)phenyl)amino)-5-methylisoxazole-3-carboxylic Acid (56)

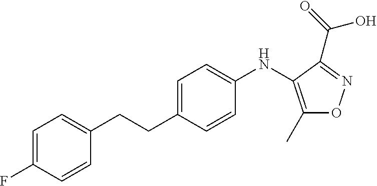 [SMILES: Cc1onc(C(=O)O)c1Nc1ccc(CCc2ccc(F)cc2)cc1]

Step a: To a solution of methyl 4-((4-((4-fluorophenyl)ethynyl)phenyl)amino)-5-methylisoxazole-3-carboxylate (Example 55, Step C₃ product) (0.160 g, 0.457 mmol) in THF (10 mL) was added 10% Pd/C (0.08 g). Hydrogen gas was purged in to the reaction mixture for 3 h at ambient temperature. The resulting reaction mixture was filtered through celite hyflow and washed with THF (50 mL). The resulting filtrate was concentrated under reduced pressure and dried to yield methyl 4-((4-(4-fluorophenethyl)phenyl)amino)-5-methylisoxazole-3-carboxylate (0.1 g, 0.282 mmol). LCMS: Method B, 4.832 min, MS: ES+355.5 (M+1).

Step b: The title compound was synthesized by hydrolysis of methyl 4-((4-(4-fluorophenethyl)phenyl)amino)-5-methylisoxazole-3-carboxylate following a similar synthetic procedure as mentioned for step e of Example 25. Compound 56: LCMS: Method B, 4.440 min, MS: ES+ 341.4 (M+1); ¹H NMR (400 MHz, DMSO-d₆) δ ppm: 13.85 (br s, 1H), 7.17-7.31 (m, 2H), 7.03-7.16 (m, 2H), 6.97 (d, J=7.2 Hz, 2H), 6.49 (d, J=6.8 Hz, 2H), 2.65-2.85 (m, 4H), 2.29 (s, 3H).

### Example 57. 4-((4′-cyano-[1,1′-biphenyl]-4-yl)(4-methoxybenzyl)amino)-5-methyl-1H-pyrazole-3-carboxylic Acid (57)

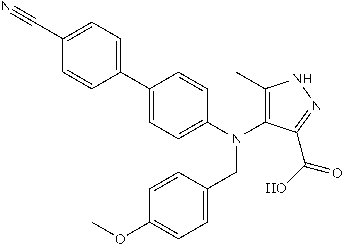 [SMILES: COc1ccc(CN(c2ccc(-c3ccc(C#N)cc3)cc2)c2c(C(=O)O)n[nH]c2C)cc1]

Compound 57 was isolated via the preparative HPLC purification as described in Example 39 and characterized as follows: LCMS: Method B, 4.440 min, MS: ES+ 439.4 (M+1).

### Example 58. 4-((4′-cyano-[1,1′-biphenyl]-3-yl)(4-methoxybenzyl)amino)-5-methyl-1H-pyrazole-3-carboxylic Acid (58)

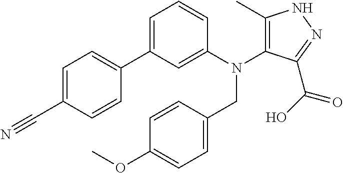 [SMILES: COc1ccc(CN(c2cccc(-c3ccc(C#N)cc3)c2)c2c(C(=O)O)n[nH]c2C)cc1]

Compound 58 was isolated via preparative HPLC purification as described in Example 42 and characterized as follows: LCMS: Method B, 4.451 min, MS: ES+ 439.4 (M+1).

### Example 59. 4-((4-(4-(2-((1-(tert-butoxycarbonyl)azetidin-3-yl)oxy)ethoxy)benzyl)phenyl) amino)-5-methyl-1H-pyrazole-3-carboxylic Acid (59)

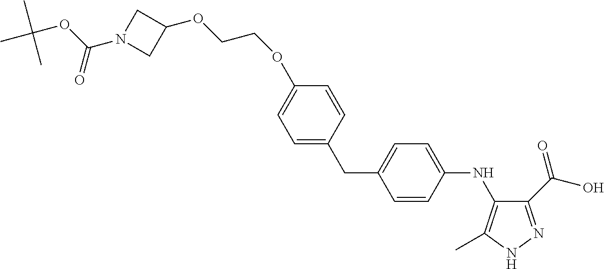 [SMILES: Cc1[nH]nc(C(=O)O)c1Nc1ccc(Cc2ccc(OCCOC3CN(C(=O)OC(C)(C)C)C3)cc2)cc1]

### Synthesis of tert-butyl 3-(2-(4-(4-bromobenzyl)phenoxy)ethoxy)azetidine-1-carboxylate (Intermediate F)

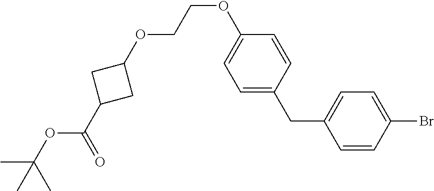 [SMILES: CC(C)(C)OC(=O)C1CC(OCCOc2ccc(Cc3ccc(Br)cc3)cc2)C1]

Step a: To a stirred solution of tert-butyl 3-(2-hydroxyethoxy)-azetidine-1-carboxylate (CAS Number 1146951-82-7) (1.1 g, 5.059 mmol) in dichloromethane (20 mL) were added DMAP (0.74 g, 6.070 mmol) and p-toluenesulfonyl chloride (CAS Number 98-59-9) (1.0 g, 6.070 mmol) at ambient temperature. The reaction mixture was stirred for 2 h. The resulting reaction mixture was poured into water (200 mL) and extracted with EtOAc (3×100 mL). The combined organic phase was dried over Na₂SO₄, filtered, concentrated under reduced pressure and dried to yield tert-butyl 3-(2-(tosyloxy)ethoxy)azetidine-1-carboxylate (1.6 g, 4.312 mmol). LCMS: Method B, 4.508 min, MS: ES+ 372.1 (M+1).

Step b: To a stirred solution of tert-butyl 3-(2-(tosyloxy)ethoxy)azetidine-1-carboxylate (1.7 g, 4.671 mmol) and 4-(4-bromobenzyl)phenol (1.2 g, 4.580 mmol) in DMF (20 mL) was added NaOH (0.55 g, 13.740 mmol) at ambient temperature. The reaction mixture was heated at 100° C. for 4 h. The resulting reaction mixture was poured into water (100 mL) and extracted with EtOAc (3×100 mL). The combined organic phase was dried over Na₂SO₄, filtered and concentrated under reduced pressure. The resulting crude material was purified by column chromatography (20% EtOAc in hexane) yielding tert-butyl 3-(2-(4-(4-bromobenzyl)phenoxy)ethoxy)azetidine-1-carboxylate (1.3 g, 2.819 mmol). LCMS: Method B, 5.148 min, MS: ES+ 362 (M−100).

The title compound, Compound 59, was synthesized via steps d₃, f & g of Scheme 10, following similar synthetic procedures as mentioned for Example 45, using tert-butyl 3-(2-(4-(4-bromobenzyl)phenoxy)ethoxy)azetidine-1-carboxylate (Intermediate F) instead of 4-bromobenzonitrile & ethyl 4-amino-1-benzyl-5-methyl-1H-pyrazole-3-carboxylate (synthesized via step a, b & c of Scheme 10, following similar synthetic procedures as mentioned for Example 39, using benzyl bromide instead of 1-(chloromethyl)-4-methoxybenzene in step b) instead of ethyl 4-amino-1-(4-methoxybenzyl)-5-methyl-1H-pyrazole-3-carboxylate in step d₃. LCMS: Method B, 4.474 min, MS: ES+ 523.1 (M+1); ¹H NMR (400 MHz, DMSO-d₆) δ ppm: 13.05 (br s, 1H), 7.09 (d, J=8.4 Hz, 2H), 6.92 (d, J=8.4 Hz, 2H), 6.83 (d, J=8.4 Hz, 2H), 6.48 (d, J=8.0 Hz, 2H), 4.29-4.46 (m, 1H), 3.98-4.10 (m, 4H), 3.71 (s, 2H), 3.60-3.70 (m, 4H), 1.99 (s, 3H), 1.37 (s, 9H).

### Example 60. 4-((4-(4-(2-(azetidin-3-yloxy)ethoxy)benzyl)phenyl)amino)-5-methyl-1H-pyrazole-3-carboxylic Acid hydrochloride (60)

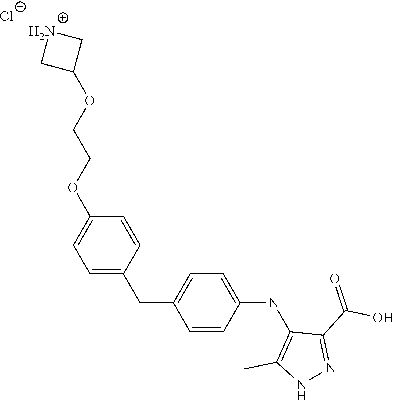 [SMILES: [Cl-] ; Cc1[nH]nc(C(=O)O)c1Nc1ccc(Cc2ccc(OCCOC3C[NH2+]C3)cc2)cc1]

To a stirred solution of 4-((4-(4-(2-((1-(tert-butoxycarbonyl)azetidin-3-yl)oxy)ethoxy)benzyl)phenyl) amino)-5-methyl-1H-pyrazole-3-carboxylic Acid (0.040 g, 0.0766 mmol) in dichloromethane (5 mL) was added 4M HCl in 1,4-dioxane (4.0 mL) at ambient temperature. The reaction mixture was stirred at ambient temperature for 4 h. The resulting reaction mixture was concentrated under reduced pressure yielding 4-((4-(4-(2-(azetidin-3-yloxy)ethoxy)benzyl)phenyl)amino)-5-methyl-1H-pyrazole-3-carboxylic acid hydrochloride (0.015 g, 0.033 mmol). LCMS: Method B, 3.924 min, MS: ES+ 423 (M+1); ¹H NMR (400 MHz, DMSO-d₆) δ ppm: 13.05 (br s, 1H), 8.99 (s, 1H), 7.10 (d, J=8.4 Hz, 2H), 6.92 (d, J=8.4 Hz, 2H), 6.84 (d, J=8.4 Hz, 2H), 6.48 (d, J=8.0 Hz, 2H), 4.41-4.49 (m, 1H), 4.08-4.18 (m, 2H), 4.03 (t, J=4.0 Hz, 2H), 3.78-3.87 (m, 2H), 3.74 (t, J=4.4 Hz, 2H), 3.71 (s, 2H), 1.99 (s, 3H).

### Example 61. 4-((4-(4-(2-(1-(tert-butoxycarbonyl)piperidin-4-yl)ethoxy)benzyl)phenyl) amino)-5-methyl-1H-pyrazole-3-carboxylic Acid (61)

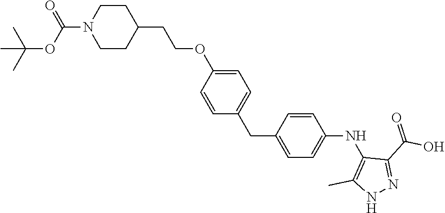 [SMILES: Cc1[nH]nc(C(=O)O)c1Nc1ccc(Cc2ccc(OCCC3CCN(C(=O)OC(C)(C)C)CC3)cc2)cc1]

Tert-butyl 4-(2-(4-(4-bromobenzyl)phenoxy)ethyl)piperidine-1-carboxylate, Intermediate G

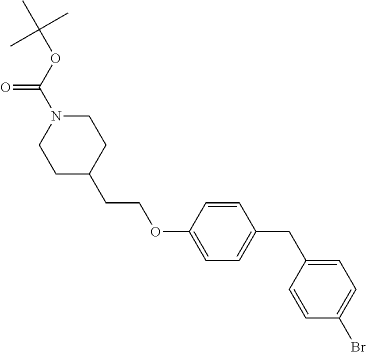 [SMILES: CC(C)(C)OC(=O)N1CCC(CCOc2ccc(Cc3ccc(Br)cc3)cc2)CC1]

The Intermediate G was synthesized following similar synthetic procedures as mentioned for tert-butyl 3-(2-(4-(4-bromobenzyl)phenoxy)ethoxy)azetidine-1-carboxylate (Intermediate F), using tert-butyl 4-(2-hydroxyethyl)piperidine-1-carboxylate (CAS Number 89151-44-0) in step a instead of tert-butyl 3-(2-hydroxyethoxy)azetidine-1-carboxylate. LCMS: Method B, 5.415 min, MS: ES+ 374 (M−100).

Compound 61 was synthesized via steps d₃, f & g of Scheme 10, following similar synthetic procedures as mentioned for Example 45, using tert-butyl 4-(2-(4-(4-bromobenzyl)phenoxy)ethyl)piperidine-1-carboxylate (Intermediate G) instead of 4-bromobenzonitrile & ethyl 4-amino-1-benzyl-5-methyl-1H-pyrazole-3-carboxylate (synthesized via step a, b & c of Scheme 10, following similar synthetic procedures as mentioned for Example 39, using benzyl bromide instead of 1-(chloromethyl)-4-methoxybenzene in step b) instead of ethyl 4-amino-1-(4-methoxybenzyl)-5-methyl-1H-pyrazole-3-carboxylate in step d₃. LCMS: Method A, 2.716 min, MS: ES− 532.9 (M−1); ¹H NMR (400 MHz, DMSO-d₆) δ ppm: 12.87 (bdr s, 1H), 7.07 (d, J=8.4 Hz, 2H), 6.92 (d, J=8.4 Hz, 2H), 6.81 (J=8.4 Hz, 2H), 6.48 (d, J=8.4 Hz, 2H), 3.85-4.00 (m, 4H), 3.70 (s, 2H), 2.6-2.75 (m, 2H), 1.99 (s, 3H), 1.55-1.63 (m, 5H), 1.38 (s, 9H), 0.95-1.10 (m, 2H).

### Example 62. 5-methyl-4-((4-(4-(2-(piperidin-4-yl)ethoxy)benzyl)phenyl)amino)-1H-pyrazole-3-carboxylic Acid hydrochloride (62)

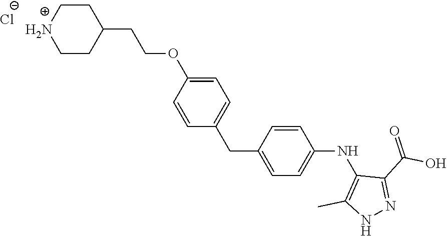 [SMILES: [Cl-] ; Cc1[nH]nc(C(=O)O)c1Nc1ccc(Cc2ccc(OCCC3CC[NH2+]CC3)cc2)cc1]

The title compound was synthesized by Boc-deprotection of Compound 61 following a similar synthetic procedure as mentioned for Example 60. LCMS: Method A, 1.736 min, MS: ES+ 435.20 (M+1); ¹H NMR (400 MHz, DMSO-d₆) δ ppm: 13.05 (br s, 1H), 8.80 (br s, 1H), 7.08 (d, J=7.6 Hz, 2H), 6.92 9d, J=8.0, 2H), 6.82 (d, J=8.0 Hz, 2H), 6.48 (d, J=7.6 Hz, 2H), 3.90-4.00 (m, 2H), 3.70 (s, 2H), 3.10-3.25 (m, 2H), 2.78-2.90 (m, 2H), 1.99 (s, 3H), 1.60-1.90 (m, 5H), 1.3-1.4 (m, 2H).

### Example 63. 4-((4-(4-(2-(4-(tert-butoxycarbonyl)piperazin-1-yl)ethoxy)benzyl)phenyl) amino)-5-methyl-1H-pyrazole-3-carboxylic Acid (63)

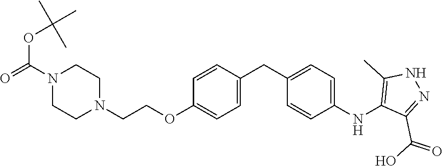 [SMILES: Cc1[nH]nc(C(=O)O)c1Nc1ccc(Cc2ccc(OCCN3CCN(C(=O)OC(C)(C)C)CC3)cc2)cc1]

### Tert-butyl 4-(2-(4-(4-bromobenzyl)phenoxy)ethyl)piperazine-1-carboxylate (Intermediate H)

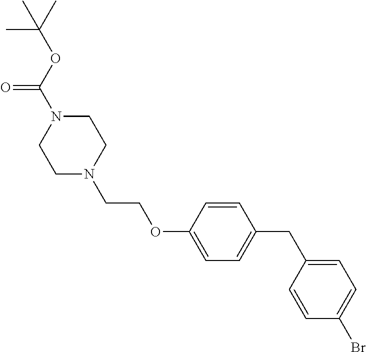 [SMILES: CC(C)(C)OC(=O)N1CCN(CCOc2ccc(Cc3ccc(Br)cc3)cc2)CC1]

Step a: To a stirred solution of 4-(4-bromobenzyl) phenol (2.00 g, 7.633 mmol) in THF (20 mL) were added 1, 2-dibromoethane (CAS Number 106-93-4) (2.12 g, 11.437 mmol) and NaOH (0.17 g, 916.03 mmol) at ambient temperature. The reaction mixture was stirred at 70° C. for 6 h. The resulting reaction mixture was poured into water (100 mL) and extracted with EtOAc (2×80 mL). The combined organic phase was dried over Na₂SO₄, filtered and concentrated under reduced pressure. The resulting crude material was purified by flash chromatography (1.5% EtOAc in hexane) yielding 1-bromo-4-(4-(2-bromoethoxy)benzyl)benzene (1.5 g, 4.348 mmol). ¹H NMR (400 MHz, DMSO-d₆) δ ppm: 7.46 (d, J=8.4 Hz, 2H), 7.290-7.070 (m, 4H), 6.88 (d, J=8.8 Hz, 2H), 4.27 (t, J=5.2 Hz, 2H), 3.85 (s, 2H), 3.78 (t, J=5.6 Hz, 2H).

Step b: To a stirred solution of 1-bromo-4-(4-(2-bromoethoxy)benzyl)benzene (1.1 g, 2.997 mmol) in acetonitrile (25 mL) were added 1-Boc-piperazine (CAS Number 57260-71-6) (0.60 g, 3.296 mmol) and K₂CO₃ (1.24 g, 8.99 mmol) at ambient temperature. The reaction mixture was heated at 100° C. for 3 h. The resulting reaction mixture was poured into water (70 mL) and extracted with EtOAc (2×40 mL). The combined organic phase was dried over Na₂SO₄, filtered and concentrated under reduced pressure. The resulting crude material was purified by flash chromatography (15.0% EtOAc in hexane) yielding tert-butyl 4-(2-(4-(4-bromobenzyl)phenoxy)ethyl)piperazine-1-carboxylate, Intermediate H (1.0 g, 2.101 mmol). LCMS: Method B, 5.194 min, MS: ES+ 475.0 (M+1).

Compound 63 was synthesized via steps d₃, f & g of Scheme 10, following similar synthetic procedures as mentioned for Example 45, using tert-butyl 4-(2-(4-(4-bromobenzyl)phenoxy)ethyl)piperazine-1-carboxylate (Intermediate H) instead of 4-bromobenzonitrile & ethyl 4-amino-1-benzyl-5-methyl-1H-pyrazole-3-carboxylate (synthesized via steps a, b & c of Scheme 10, following similar synthetic procedures as mentioned for Example 39, using benzyl bromide instead of 1-(chloromethyl)-4-methoxybenzene in step b) instead of ethyl 4-amino-1-(4-methoxybenzyl)-5-methyl-1H-pyrazole-3-carboxylate in step d3. LCMS: Method A, 1.913 min, MS: ES+ 436.01 (M+1).

### Example 64. 5-methyl-4-((4-(4-(2-(piperazin-1-yl)ethoxy)benzyl)phenyl)amino)-1H-pyrazole-3-carboxylic Acid hydrochloride (64)

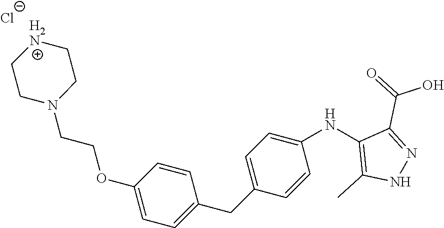

The title compound was synthesized by Boc-deprotection of Compound 63 following similar synthetic procedure as mentioned for Example 60. LCMS: Method A, 1.559 min, MS: ES+ 436.11 (M+1).

### Example 65. 5-methyl-4-((4-(4-(4-morpholinobutoxy)benzyl)phenyl)amino)-1H-pyrazole-3-carboxylic Acid (65)

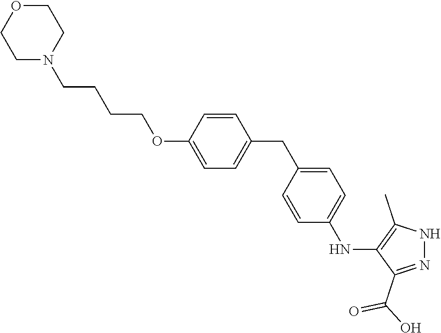 [SMILES: Cc1[nH]nc(C(=O)O)c1Nc1ccc(Cc2ccc(OCCCCN3CCOCC3)cc2)cc1]

### 4-(4-(4-(4-bromobenzyl)phenoxy)butyl)morpholine (Intermediate I)

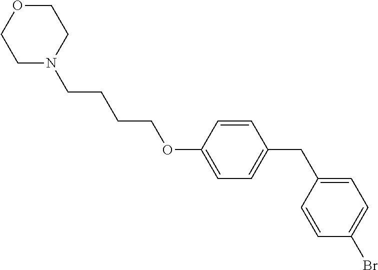 [SMILES: Brc1ccc(Cc2ccc(OCCCCN3CCOCC3)cc2)cc1]

Intermediate I was synthesized following similar synthetic procedures as mentioned for tert-butyl 4-(2-(4-(4-bromobenzyl)phenoxy)ethyl)piperazine-1-carboxylate (Intermediate H), using 1,4-dibromobutane in step a instead of 1,2-dibromoethane and morpholine in step b instead of tert-butyl piperazine-1-carboxylate. LCMS: Method B, 5.120 min, MS: ES+ 404 (M+1).

Compound 65 was synthesized via step d3, f & g of Scheme 10, following similar synthetic procedures as mentioned for Example 45, using 4-(4-(4-(4-bromobenzyl)phenoxy)butyl)morpholine (Intermediate I) instead of 4-bromobenzonitrile & ethyl 4-amino-1-(4-methoxybenzyl)-5-methyl-1H-pyrazole-3-carboxylate (step c product, Example 39) instead of ethyl 4-amino-1-(4-methoxybenzyl)-3-methyl-1H-pyrazole-5-carboxylate in step d₃. LCMS: Method A, 1.736 min, MS: ES+ 465.15 (M+1); ¹H NMR (400 MHz, DMSO-d₆) δ ppm: 12.87 (br s, 1H), 7.07 (d, J=8.8 Hz, 1H), 6.92 (d, J=8.4 Hz, 2H), 6.81 (d, J=8.8 Hz, 2H), 6.49 (d, J=8.4 Hz, 2H), 3.92 (t, 6.8 Hz, 2H), 3.71 (s, 2H), 3.55 (t, J=4.4 Hz, 4H), 5.27-5.40 (m, 6H), 1.99 (s, 3H), 1.66-1.73 (m, 2H), 1.53-1.58 (m, 2H).

### Example 66. 4-((4-(4-Fluorophenethyl)phenyl)amino)-3-methyl-1H-pyrazole-5-carboxylic Acid (66)

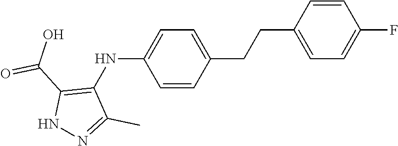 [SMILES: Cc1n[nH]c(C(=O)O)c1Nc1ccc(CCc2ccc(F)cc2)cc1]

Synthesis of ethyl 4-((4-(4-fluorophenethyl)phenyl)amino)-1-(4-methoxybenzyl)-3-methyl-1H-pyrazole-5-carboxylate: To a solution of ethyl 4-((4-((4-fluorophenyl)ethynyl)phenyl)amino)-1-(4-methoxybenzyl)-3-methyl-1H-pyrazole-5-carboxylate (synthesized via steps a, b, c & d3 of Scheme 10, following similar synthetic procedures as mentioned for Example 45, using 1-bromo-4-((4-fluorophenyl)ethynyl)benzene (CAS Number 64583-19-3) in step d₃ instead of 4-bromobenzonitrile) (0.4 g, 0.828 mmol) in THF (20 mL) was added 10% Pd/C (0.2 g). The reaction mixture was stirred under hydrogen atmosphere (200 psi) at ambient temperature. The resulting reaction mixture was filtered through celite hyflow and washed with THF (100 mL). The resulting filtrate was concentrated under reduced pressure and dried yielding ethyl 4-((4-(4-fluorophenethyl)phenyl)amino)-1-(4-methoxybenzyl)-3-methyl-1H-pyrazole-5-carboxylate (0.3 g, 0.614 mmol). LCMS: Method B, 5.069 min, MS: ES+ 488.5 (M+1).

The title compound was synthesized via steps f & g of Scheme 10, following similar synthetic procedures as mentioned for Example 39, using ethyl 4-((4-(4-fluorophenethyl)phenyl)amino)-1-(4-methoxybenzyl)-3-methyl-1H-pyrazole-5-carboxylate in step f instead of ethyl 4-((4′-cyano-[1,1′-biphenyl]-4-yl)amino)-1-(4-methoxybenzyl)-5-methyl-1H-pyrazole-3-carboxylate. During this synthesis formation of compound 67 was also observed as a by-product which was also isolated and characterized as mentioned below. Compound 66: LCMS: Method B, 4.397 min, MS: ES+ 340.59 (M+1); ¹H NMR (400 MHz, DMSO-d₆) δ ppm: 7.11-7.20 (m, 2H), 6.91-7.05 (m, 4H), 6.59 (d, J=7.6 Hz, 2H), 2.70-2.90 (m, 4H), 2.10 (s, 3H).

### Example 67. 4-((4-(4-Fluorophenethyl)phenyl)(4-methoxybenzyl)amino)-3-methyl-1H-pyrazole-5-carboxylic Acid (67)

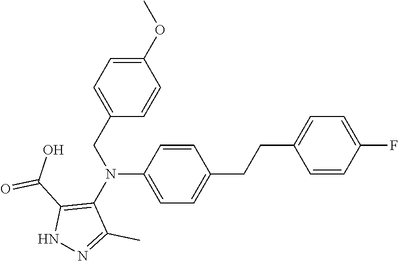 [SMILES: COc1ccc(CN(c2ccc(CCc3ccc(F)cc3)cc2)c2c(C)n[nH]c2C(=O)O)cc1]

LCMS: Method B, 4.710 min, MS: ES+ 460.7 (M+1)

### Example 68. 4-((4-(4-(2-((1-(tert-butoxycarbonyl)azetidin-3-yl)oxy)ethoxy)benzyl)phenyl)amino)pyridazine-3-carboxylic Acid sodium Salt (68)

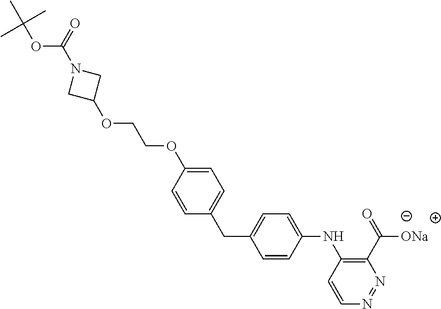

### tert-Butyl 3-(2-(4-(4-aminobenzyl)phenoxy)ethoxy)azetidine-1-carboxylate (Intermediate J)

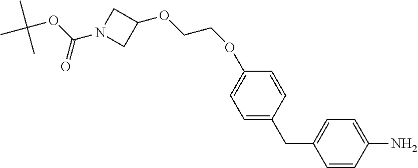 [SMILES: CC(C)(C)OC(=O)N1CC(OCCOc2ccc(Cc3ccc(N)cc3)cc2)C1]

Step a: To a stirred solution of 5-hydroxy-2-(4-nitrobenzyl)benzene-1-ylium (0.5 g, 2.0181 mmol) and ethyl tert-butyl 3-(2-hydroxyethoxy)-azetidine-1-carboxylate (CAS Number 1146951-82-7) (1.1 g, 5.453 mmol) in THF (20 mL) were added TPP (CAS Number 115-86-6) (1.5 g, 5.453 mmol) and DIAD (CAS Number 2446-83-5) (1.1 g, 5.453 mmol) at 0-5° C. under nitrogen atmosphere. The reaction mixture was stirred at ambient temperature for 4 h. The resulting reaction mixture was poured into water (100 mL) and extracted with EtOAc (3×100 mL). The combined organic phase was dried over Na₂SO₄, filtered and concentrated under reduced pressure. The resulting solid was dried yielding tert-butyl 3-(2-(4-(4-nitrobenzyl)phenoxy)ethoxy)azetidine-1-carboxylate (0.35 g, 0.817 mmol). LCMS: Method B, 4.914 min, MS: ES+ 329.1 (M−100).

Step b: To a solution of tert-butyl 3-(2-(4-(4-nitrobenzyl)phenoxy)ethoxy)azetidine-1-carboxylate (0.65 g, 1.518 mmol) in MeOH (10 mL) was added 10% Pd/C (0.3 g). Hydrogen gas was purged in to the reaction mixture for 4 h at ambient temperature. The resulting reaction mixture was filtered through celite hyflow and washed with MeOH (30 mL). The resulting filtrate was concentrated under reduced pressure and dried yielding tert-butyl 3-(2-(4-(4-aminobenzyl)phenoxy)ethoxy)azetidine-1-carboxylate (Intermediate J) (0.6 g, 1.507 mmol). LCMS: Method B, 4.661 min, MS: ES+ 399.1 (M+1).

Compound 68 was synthesized via steps a, b, c, d, e & f of Scheme 12, following similar synthetic procedures as mentioned for Example 47, using tert-butyl 3-(2-(4-(4-aminobenzyl)phenoxy)ethoxy)azetidine-1-carboxylate (Intermediate J) in step d instead of 4-fluoro aniline. LCMS: Method B, 4.438 min, MS: ES+ 521.13 (M+1); ¹H NMR (400 MHz, DMSO-d₆) δ ppm: 12.32 (s, 1H), 8.53 (d, J=6.0 Hz, 1H), 7.0-7.3 (m, 6H), 6.87 (d, J=8.4 Hz, 2H), 4.39-4.48 (m, 1H), 3.98-4.10 (m, 4H), 3.86 (s, 2H), 3.60-3.75 (m, 4H), 1.37 (s, 9H).

### Example 69. 4-((4-(4-(2-(Azetidin-3-yloxy)ethoxy)benzyl)phenyl)amino)-pyridazine-3-carboxylic Acid hydrochloride (69)

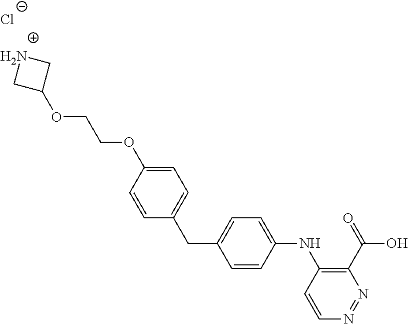 [SMILES: [Cl-] ; O=C(O)c1nnccc1Nc1ccc(Cc2ccc(OCCOC3C[NH2+]C3)cc2)cc1]

To a stirred solution of 4-((4-(4-(2-((1-(tert-butoxycarbonyl)azetidin-3-yl)oxy)ethoxy)benzyl)phenyl)amino)pyridazine-3-carboxylic acid sodium salt (0.045 g, 0.0828 mmol) in DCM (5 mL) was added 4 M HCl in dioxane (2.0 mL) at ambient temperature. The reaction mixture was stirred at ambient temperature for 4 h. The resulting reaction mixture was concentrated under reduced pressure yielding 4-((4-(4-(2-(azetidin-3-yloxy)ethoxy)benzyl)phenyl)amino)pyridazine-3-carboxylic acid hydrochloride (0.010 g, 0.023 mmol). LCMS: Method E, 1.884 min, MS: ES+ 421.27 (M+1); ¹H NMR (400 MHz, DMSO-d₆) δ ppm: 9.09 (br s, 1H), 8.72 (m, 1H), 7.10-7.45 (m, 6H), 6.81-6.93 (m, 2H), 6.56 (s, 1H), 4.40-4.50 (m, 1H), 4.05-4.20 (m, 4H), 3.91 (s, 1H), 3.70-3.88 (m, 4H).

### Example 70. 4-((4-(4-(2-(1-(tert-Butoxycarbonyl)piperidin-4-yl)ethoxy)benzyl)phenyl) amino)pyridazine-3-carboxylic Acid (70)

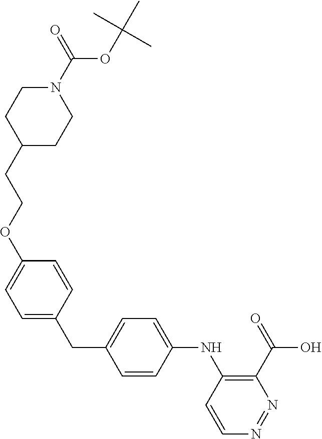 [SMILES: CC(C)(C)OC(=O)N1CCC(CCOc2ccc(Cc3ccc(Nc4ccnnc4C(=O)O)cc3)cc2)CC1]

### tert-Butyl 4-(2-(4-(4-aminobenzyl)phenoxy)ethyl)piperidine-1-carboxylate (Intermediate M)

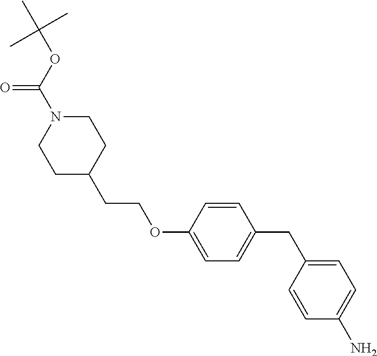 [SMILES: CC(C)(C)OC(=O)N1CCC(CCOc2ccc(Cc3ccc(N)cc3)cc2)CC1]

Intermediate M was synthesized following similar synthetic procedures as mentioned for tert-butyl 3-(2-(4-(4-aminobenzyl)phenoxy)ethoxy)azetidine-1-carboxylate (Intermediate J), using tert-butyl 4-(2-hydroxyethyl)piperidine-1-carboxylate (CAS Number 89151-44-0) in step a instead of tert-butyl 3-(2-hydroxyethoxy)azetidine-1-carboxylate. LCMS: Method A, 2.523 min, MS: ES+ 311.2 (M−100).

The title compound was synthesized via step a, b, c, d, e & f of Scheme 12, following similar synthetic procedures as mentioned for Example 47, using tert-butyl 4-(2-(4-(4-aminobenzyl)phenoxy)ethyl)piperidine-1-carboxylate (Intermediate M) in step d instead of 4-fluoro aniline. Compound 70, Na salt: LCMS: Method A, 2.391 min, MS: ES+ 533.2 (M+1); ¹H NMR (400 MHz, DMSO-d₆) δ ppm: 12.32 (s, 1H), 8.52 (d, J=6.0 Hz, 1H), 7.05-7.30 (m, 7H), 6.85 (d, J=8.4 Hz, 2H), 3.9-4.0 (m, 4H), 3.85 (s, 2H), 2.6-2.75 (m, 2H), 1.56-1.70 (m, 5H), 1.38 (s, 9H), 0.95-1.10 (m, 2H). Compound 70 free acid: LCMS: Method A, 2.382 min, MS: ES+ 533.11 (M+1).

### Example 71. 4-((4-(4-(2-(Piperidin-4-yl)ethoxy)benzyl)phenyl)amino)-pyridazine-3-carboxylic Acid hydrochloride (71)

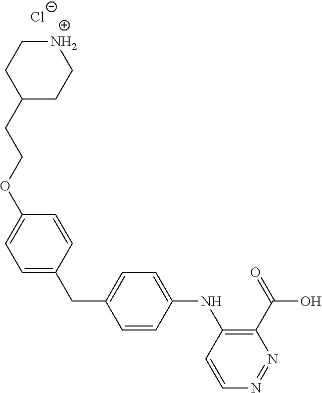 [SMILES: [Cl-] ; O=C(O)c1nnccc1Nc1ccc(Cc2ccc(OCCC3CC[NH2+]CC3)cc2)cc1]

The title compound was synthesized by Boc-deprotection of Compound 70 following similar synthetic procedure as mentioned for Example 69. LCMS: Method A, 1.585 min, MS: ES+ 433.2 (M+1); ¹H NMR (400 MHz, DMSO-d₆) δ ppm: 11.08 (br s, 1H). 8.92 (s, 1H), 8.69 (s, 1H), 7.25-7.40 (m, 4H), 7.10-7.20 (m, 2H), 6.86 (d, J=8.4 Hz, 2H), 3.97 (t, J=6.0 Hz, 2H), 3.92 (s, 2H), 3.18-3.29 (m, 2H), 2.75-2.90 (m, 2H), 1.6-1.9 (m, 5H), 1.31-1.45 (m, 2H).

### Example 72. 4-((4-(4-(2-(4-(tert-Butoxycarbonyl)piperazin-1-yl)ethoxy)-benzyl)phenyl)amino)pyridazine-3-carboxylic Acid Sodium Salt (72)

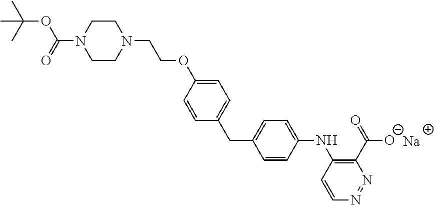 [SMILES: CC(C)(C)OC(=O)N1CCN(CCOc2ccc(Cc3ccc(Nc4ccnnc4C(=O)[O-])cc3)cc2)CC1 ; [Na+]]

### tert-Butyl 4-(2-(4-(4-aminobenzyl)phenoxy)ethyl)piperazine-1-carboxylate (Intermediate N)

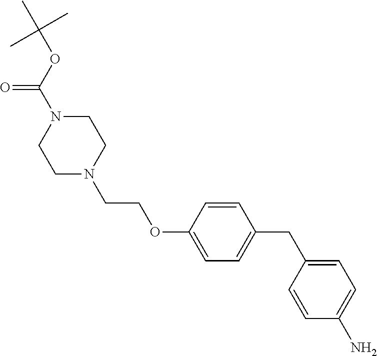 [SMILES: CC(C)(C)OC(=O)N1CCN(CCOc2ccc(Cc3ccc(N)cc3)cc2)CC1]

Step a: To a stirred solution 4-(4-nitrobenzyl)phenol (Synthesized as per Journal of Medicinal Chemistry; vol. 58; nb. 12; (2015); p. 5096-5107) (1.50 g, 6.550 mmol) in THF (20 mL) were added 1, 2-dibromoethane (CAS Number 106-93-4) (1.35 g, 7.205 mmol) and NaOH (0.78 g, 19.650 mmol) at ambient temperature. The reaction mixture was stirred at 70° C. for 12 hour. The resulting reaction mixture was poured into water (100 mL) and extracted with EtOAc (2×80 mL). The combined organic phase was dried over Na₂SO₄, filtered and concentrated under reduced pressure. The resulting crude material was purified by flash chromatography (3.0% EtOAc in hexane) yielding 1-(2-bromoethoxy)-4-(4-nitrobenzyl)benzene (0.9 g, 4.348 mmol). ¹H NMR (400 MHz, DMSO-d₆) δ ppm: 8.12-8.20 (m, 2H), 7.49 (d J=8.8 Hz, 2H), 7.19 (d, J=8.4 Hz, 2H), 6.88-6.94 (m, 2H), 4.28 (t, J=5.2 Hz, 2H), 378 (t, J=5.2 Hz, 2H).

Step b: To a stirred solution of 1-(2-bromoethoxy)-4-(4-nitrobenzyl)benzene (0.85 g, 2.537 mmol) in acetonitrile (25 mL) were added 1-Boc-piperazine (CAS Number 57260-71-6) (0.51 g, 2.790 mmol) and K₂CO₃ (1.05 g, 7.610 mmol) at ambient temperature. The reaction mixture was stirred at 100° C. for 3 h. The resulting reaction mixture was poured into water (70 mL) and extracted with EtOAc (2×40 mL). The combined organic phase was dried over Na₂SO₄, filtered and concentrated under reduced pressure. The resulting crude material was purified by flash chromatography (26.5% EtOAc in hexane) yielding tert-butyl 4-(2-(4-(4-nitrobenzyl)phenoxy)ethyl)piperazine-1-carboxylate (0.850 g, 1.192 mmol). LCMS: Method B, 4.958 min, MS: ES+ 442.1 (M+1).

Step c: To a solution of tert-butyl 4-(2-(4-(4-nitrobenzyl)phenoxy)ethyl)piperazine-1-carboxylate (0.60 g, 1.35 mmol) in MeOH (10 mL) was added 10% Pd/C (0.1 g). Hydrogen gas was purged in to the reaction mixture for 4 h at ambient temperature. The resulting reaction mixture was filtered through celite hyflow and washed with MeOH (30 mL). The resulting filtrate was concentrated under reduced pressure and dried yielding tert-butyl 4-(2-(4-(4-aminobenzyl)phenoxy)ethyl)piperazine-1-carboxylate (Intermediate N). (0.45 g, 1.090 mmol). LCMS: Method B, 4.718 min, MS: ES+ 412.11 (M+1).

Compound 72 was synthesized via steps a, b, c, d, e & f of Scheme 12, following similar synthetic procedures as mentioned for Example 47, using tert-butyl 4-(2-(4-(4-aminobenzyl)phenoxy)ethyl)piperazine-1-carboxylate (Intermediate N) in step d instead of 4-fluoro aniline. LCMS: Method A, 1.649 min, MS: ES+ 534.1 (M+1); ¹H NMR (400 MHz, DMSO-d₆) δ ppm: 12.32 (s, 1H), 8.52 (d, J=6.0 Hz, 1H), 7.06-7.30 (m, 7H), 6.80-6.98 (m, 2H), 4.03 (t, J=5.6 Hz, 2H), 3.85 (s, 2H), 3.25-3.35 (m, 4H), 2.68 (t, J=5.6 Hz, 2H), 2.40-2.49 (m, 4H), 1.39 (s, 9H).

### Example 73. 4-((4-(4-(2-(Piperazin-1-yl)ethoxy)benzyl)phenyl)amino)-pyridazine-3-carboxylic Acid hydrochloride (73)

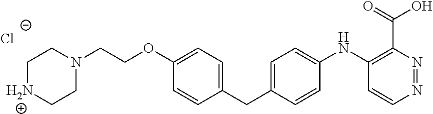 [SMILES: [Cl-] ; O=C(O)c1nnccc1Nc1ccc(Cc2ccc(OCCN3CC[NH2+]CC3)cc2)cc1]

The title compound was synthesized by Boc-deprotection of Compound 72 following similar synthetic procedure as mentioned for Example 69. LCMS: Method B, 3.877 min, MS: ES+ 434.1 (M+1); ¹H NMR (400 MHz, DMSO-d₆) δ ppm: 11.28 (br s, 1H), 9.82 (br s, 1H), 8.81 (d, J=6.8 Hz, 1H), 7.32-7.40 (m, 4H), 7.23-7.27 (m, 3H), 6.98 (d, J=8.0 Hz, 1H), 4.48 (s, 2H), 3.97 (s, 2H), 3.40-3.60 (m, 10H).

### Example 74. 4-((4-(4-(4-Morpholinobutoxy)benzyl)phenyl)amino)pyridazine-3-carboxylic Acid sodium Salt (74)

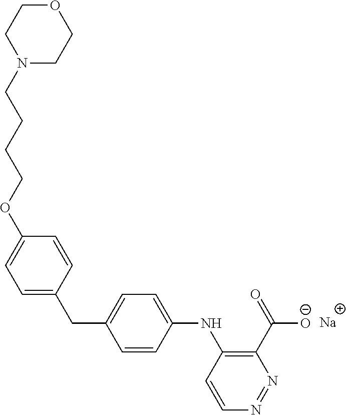 [SMILES: O=C([O-])c1nnccc1Nc1ccc(Cc2ccc(OCCCCN3CCOCC3)cc2)cc1 ; [Na+]]

### 4-(4-(4-Morpholinobutoxy)benzyl)aniline (Intermediate O)

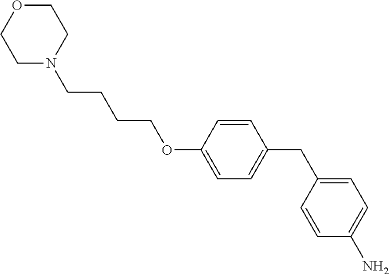 [SMILES: Nc1ccc(Cc2ccc(OCCCCN3CCOCC3)cc2)cc1]

Intermediate O was synthesized following similar synthetic procedures as mentioned for tert-butyl 4-(2-(4-(4-aminobenzyl)phenoxy)ethyl)piperazine-1-carboxylate (Intermediate N), using 1,4-dibromobutane in step a instead of 1,2-dibromoethane and morpholine in step b instead of tert-butyl piperazine-1-carboxylate. LCMS: Method E, 2.629 min, MS: ES+ 341.3 (M+1).

Compound 74 was synthesized via steps a, b, c, d, e & f of Scheme 12, following similar synthetic procedures as mentioned for Example 47, using 4-(4-(4-morpholinobutoxy)benzyl)aniline (Intermediate O) in step d instead of 4-fluoro aniline. LCMS: Method B, 4.296 min, MS: ES+ 463 (M+1); ¹H NMR (400 MHz, DMSO-d₆) δ ppm: 12.30 (s, 1H), 8.53 (d, J=6.4 Hz, 1H), 7.19-7.29 (m, 2H), 7.11-7.18 (m, 4H), 7.07-7.10 (m, 1H), 6.81-6.89 (m, 2H), 3.93 (t, J=6.4 Hz, 2H), 3.85 (s, 2H), 3.52-3.59 (m, 4H), 2.25-2.37 (m, 6H), 1.66-1.73 (m, 2H), 1.52-1.56 (m, 2H).

### Example 75. 4-((1-(4-fluorophenyl)hexyl)thio)-5-methylisoxazole-3-carboxylic Acid (75)

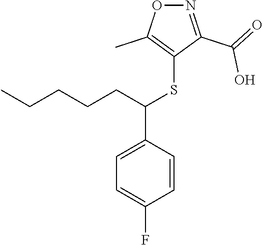 [SMILES: CCCCCC(Sc1c(C(=O)O)noc1C)c1ccc(F)cc1]

### 1-(4-fluorophenyl)hexane-1-thiol (Intermediate P)

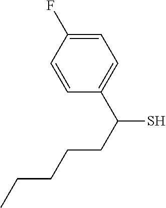

Step a: To a stirred solution of 4-fluorobenzaldehyde (CAS Number 459-57-4) (2.0 g, 16.114 mmol) in dry THF (20 mL) was added pentylmagnesium bromide (1M in THF) (19.39 mL, 19.336 mmol) at 0° C. The reaction mixture was stirred for 4 h at room temperature. The resulting reaction mixture was poured into NH₄Cl solution (70 mL) and extracted with EtOAc (2×50 mL). The combined organic phase was dried over Na₂SO₄, filtered and concentrated under reduced pressure. The resulting crude material was purified by flash chromatography (13% EtOAc in hexane) yielding 1-(4-fluorophenyl)hexan-1-ol (1.67 g, 8.516 mmol). LCMS: Method B, 4.792 min, MS: ES+ 196.5 (M+1).

Step b: To a solution of 1-(4-fluorophenyl)hexan-1-ol (0.7 g, 3.824 mmol) in toluene (15 mL) was added Lawesson's reagent (0.77 g, 1.912 mmol) at ambient temperature. The reaction mixture was heated at 80° C. for 4 h.

Another reaction on the same scale was conducted. The resulting reaction mixtures were poured into water (140 mL) and extracted with EtOAc (2×120 mL). The organic layer was washed with brine (2×140 mL). The combined organic phase was dried over Na₂SO₄, filtered and concentrated under reduced pressure. The resulting crude material was purified by flash chromatography (1% EtOAc in hexane) yielding 1-(4-fluorophenyl)hexane-1-thiol (Intermediate P) (1.16 g, 5.471 mmol) which was directly used for the synthesis of Compound 75.

Compound 75 was synthesized via steps a, b, c & d of Scheme 13, following similar synthetic procedures as mentioned for Example 51, using 1-(4-fluorophenyl)hexane-1-thiol (Intermediate P) in step b instead of phenylmethanethiol. LCMS: Method B, 4.570 min, MS: ES+ 355.5 (M+18); ¹H NMR (400 MHz, DMSO-d₆) δ ppm: 13.76 (br s, 1H), 7.17-7.22 (m, 2H), 7.07 (t, J=8.8 Hz, 2H), 4.40 (t, J=7.2 Hz, 1H), 1.93 (s, 3H), 1.83-1.88 (m, 2H), 1.10-1.37 (m, 6H), 0.79-0.82 (m, 3H).

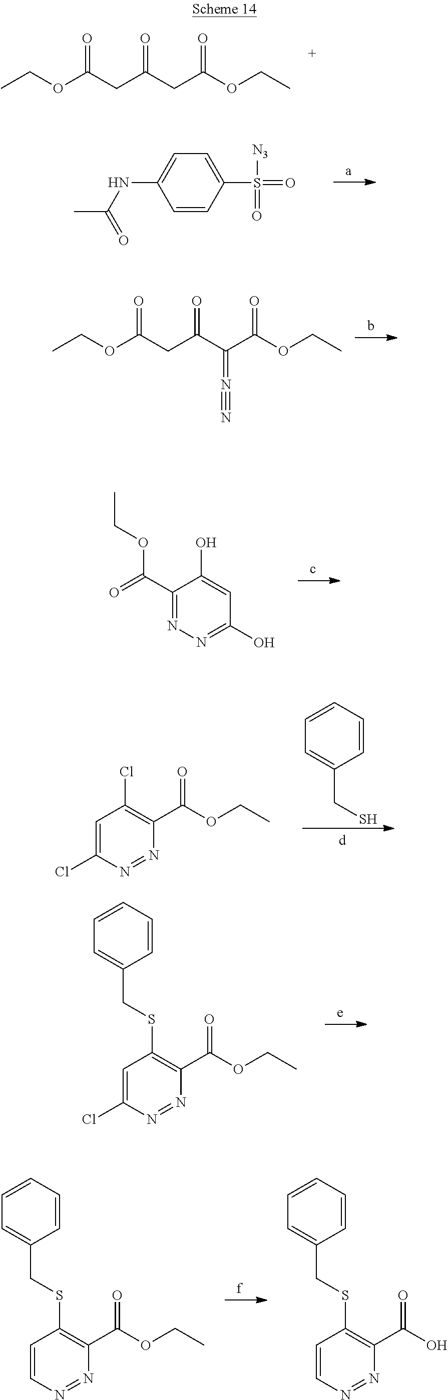

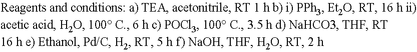

### Example 76. 4-(Benzylthio)pyridazine-3-carboxylic Acid (76)

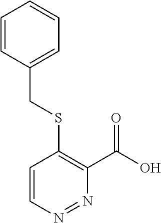 [SMILES: O=C(O)c1nnccc1SCc1ccccc1]

The title compound was prepared according to Scheme 14. Step d: To a stirred solution of ethyl 4, 6-dichloropyridazine-3-carboxylate (step c product of Example 47) (0.3 g, 1.364 mmol) in THF (5 mL) were added NaHCO₃ (0.35 g, 4.092 mmol) and benzyl mercaptam (CAS Number 100-53-8) (0.16 g, 1.364 mmol) at ambient temperature. The reaction mixture was heated at 60° C. for 16 h. The resulting reaction mixture was poured into water (100 mL) and extracted with EtOAc (3×100 mL). The combined organic phase was dried over Na₂SO₄, filtered and concentrated under reduced pressure. The resulting crude material was purified by column chromatography (20% EtOAc in hexane) yielding ethyl 4-(benzylthio)-6-chloropyridazine-3-carboxylate (0.2 g, 0.649 mmol). LCMS: Method B, 4.575 min, MS: ES+ 309.4 (M+1).

Step e: To a solution of ethyl 4-(benzylthio)-6-chloropyridazine-3-carboxylate (0.2 g, 0.649 mmol) in ethanol (10 mL) was added 10% Pd/C (0.1 g). Hydrogen gas was purged in to the reaction mixture for 4 h at ambient temperature. The resulting reaction mixture was filtered through celite hyflow and washed with Ethanol (30 mL). The resulting filtrate was concentrated under reduced pressure. The resulting crude material was purified by column chromatography (70% EtOAc in hexane) yielding ethyl 4-(benzylthio)pyridazine-3-carboxylate (0.047 g, 0.171 mmol). LCMS: Method B, 4.345 min, MS: ES+ 275.4 (M+1).

Step f: To a stirred solution of ethyl 4-(benzylthio)pyridazine-3-carboxylate (0.045 g, 0.547 mmol) in THF:water (1:1, 5 mL) was added NaOH (0.02 g, 0.349 mmol) at ambient temperature and the reaction mixture was stirred for 3 h. The resulting reaction mixture was poured into saturated NaHCO₃ solution (20 mL) and washed with dichloromethane (2×10 mL). The obtained aqueous layer was acidified (pH=4) with dilute HCl (10 mL) and extracted with dichloromethane (3×10 mL). The combined organic phase was dried over Na₂SO₄, filtered and concentrated under reduced pressure. The resulting solid was dried yielding 4-(benzylthio)pyridazine-3-carboxylic acid, compound 76 (0.015 g, 0.060 mmol). LCMS: Method B, 3.132 min, MS: ES+ 247.42 (M+1); ¹H NMR (400 MHz, DMSO-d₆) δ ppm: 14.01 (br s, 1H), 9.09 (d, J=6.0 Hz, 1H), 7.83 (d, J=6.0 Hz, 1H), 7.45-7.48 (m, 2H), 7.37 (t, J=7.2 Hz, 2H), 7.28-7.33 (m, 1H), 4.34 (s, 2H).

### Example 77. 4-((1-(4-Fluorophenyl)hexyl)thio)pyridazine-3-carboxylic Acid (77)

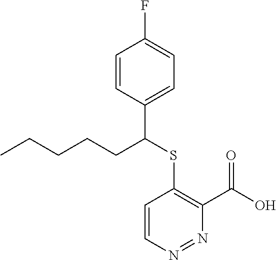 [SMILES: CCCCCC(Sc1ccnnc1C(=O)O)c1ccc(F)cc1]

The title compound was synthesized via steps a, b, c, d, e & f of Scheme 14, following similar synthetic procedures as mentioned for Example 76, using 1-(4-fluorophenyl)hexane-1-thiol (Intermediate P) in step d instead of phenylmethanethiol. During this synthesis formation of Compound 78 as a by-product was also observed, which was also isolated and characterized as mentioned below. Compound 77: LCMS: Method A, 2.621 min, MS: ES+ 334.9 (M+1).

### Example 78. (E)-4-((1-(4-Fluorophenyl)hex-1-en-1-yl)thio)-1,2-dihydropyridazine-3-carboxylic Acid (78)

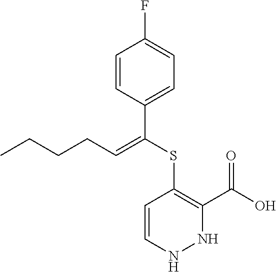 [SMILES: CCCC/C=C(/SC1=C(C(=O)O)NNC=C1)c1ccc(F)cc1]

LCMS: Method A, 2.967 min, MS: ES+ 335 (M+1); ¹H NMR (400 MHz, DMSO-d₆) δ ppm: 8.67 (d, J=2.8 Hz, 1H), 7.48-7.51 (m, 2H), 7.09-7.17 (m, 3H), 5.11-5.15 (m, 1H), 4.10-4.16 (m, 2H), 1.92-2.04 (m, 2H), 1.29-1.35 (m, 2H), 0.79-0.85 (m, 3H).

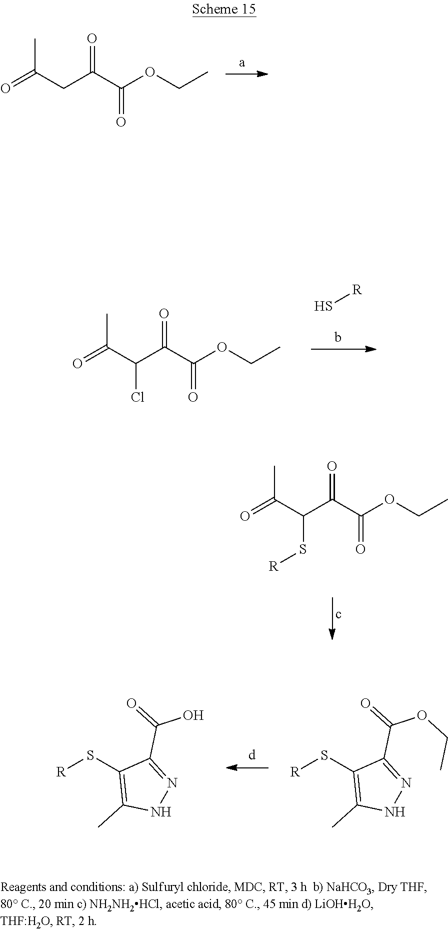

### Example 79. 4-((1-(4-Fluorophenyl)hexyl)thio)-5-methyl-1H-pyrazole-3-carboxylic Acid (79)

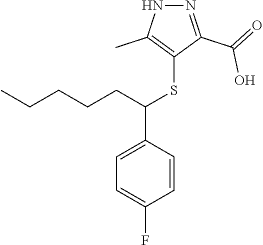 [SMILES: CCCCCC(Sc1c(C(=O)O)n[nH]c1C)c1ccc(F)cc1]

The title compound was synthesized via step a, b, c & d of Scheme 15, following similar synthetic procedures as mentioned for Example 51, using 1-(4-fluorophenyl)hexane-1-thiol (Intermediate P) in step b instead of phenylmethanethiol & hydrazine hydrate in step c instead of hydroxylamine hydrochloride.

LCMS: Method B, 4.567 min, MS: ES+ 337.5 (M+1); ¹H NMR (400 MHz, DMSO-d₆) δ ppm: 13.05 (br s, 1H), 12.74 (br s, 1H), 6.86-7.16 (m, 4H), 4.53 (s, 1H), 1.84 (s, 3H), 1.76-1.79 (m, 2H), 1.10-1.38 (m, 6H), 0.72-0.82 (m, 3H).

### Example 80. In Vitro Inhibition of Mouse Glycolate Oxidase (mGO)

Protein expression. BL21 (DE3) E. coli transformed with recombinant pET-15b expression vector containing mouse Hao1 cDNA with an N-terminal His₆ tag was grown in LB medium in the presence of 0.1 mg/mL ampicillin. For purification of recombinant mGO expressed in BL21 E. coli, bacteria pellets were thawed and re-suspended in 2 mL lysis buffer (50 mM NaH₂PO₄, 300 mM NaCl, 10 mM imidazole, 50 μM flavin mononucleotide (FMN), pH 7.5), and then treated for 30 minutes with 1 mM phenylmethylsulfonyl fluoride (PMSF) for protease inhibition, 0.1% Triton X-100 and 0.2 mg/mL lysozyme to break cellular membranes. After sonication, cells were centrifuged and the supernatant containing the total cellular extract (pre-column fraction) was loaded into a Ni-NTA agarose column, and incubated for 30 minutes at 4° C. to allow binding. The column was washed with two bed volumes of lysis buffer with 20 mM imidazole to eliminate unbound proteins (wash fraction). mGO was eluted using the same buffer containing 300 mM imidazole. Fractions containing purified GO were dialyzed against 300 mL of dialysis buffer (50 mM NaH₂PO₄, 300 mM NaCl, pH 7.5) at 4° C. with agitation overnight, and then kept at 4° C. in darkness. Protein was quantified by the bicinchoninic acid (BCA) assay.

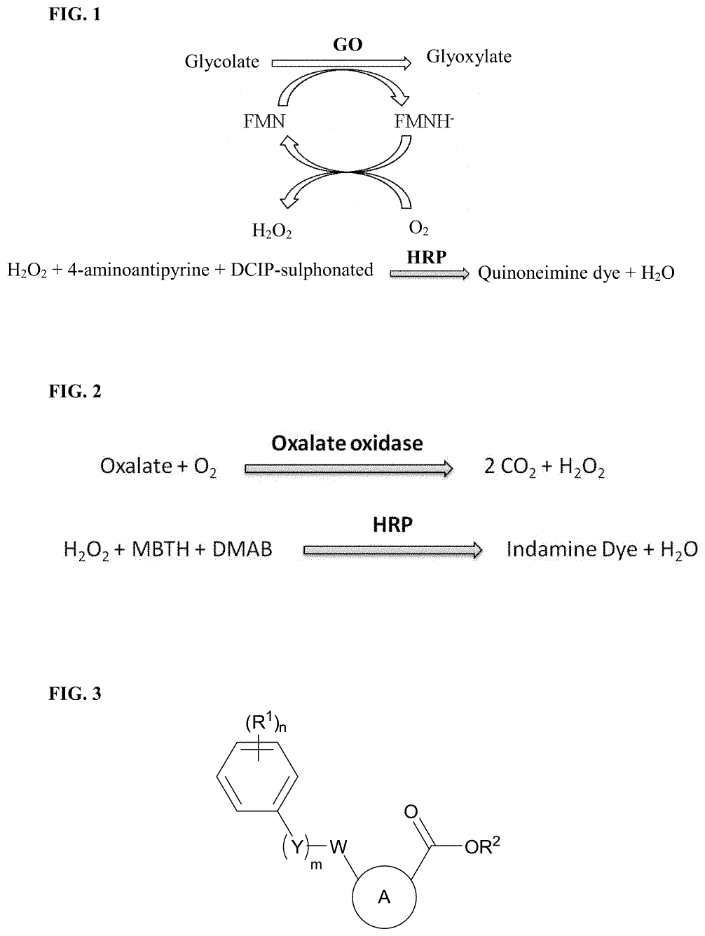

Inhibition assays. Enzymatic activity of mGO was determined in the presence of glycolate as substrate (40 mM glycolic acid) and phosphate buffer (50 mM KPO₄, 0.1 mM EDTA, pH 7). The production of glyoxylate was indirectly measured by the quantification of hydrogen peroxide formed during the first oxidation reaction. This hydrogen peroxide reacted with 4.9 mM 4-aminoantipyrine and 0.1 mM sulphonated 2,4-dichlorophenolindophenol in a coupled horseradish peroxidase (HRP) reaction that yields a quinoneimine dye (FIG. 1) measured at 515 nm (Trinder reaction). Enzymatic activity was calculated at 1 minute after initiation of the Trinder reaction. Inhibition of mGO in the presence of the test compounds (100 μM) as summarized in Table 3 below.

TABLE 3
In vitro inhibition of mouse glycolate oxidase by
compounds of the invention.

| Compound No. | % Inhibition¹ |
|---|---|
| 1 | ++ |
| 2 | + |
| 6 | ++ |
| 7 | ++ |
| 8 | + |
| 10 | ++ |
| 12 | + |
| 13 | + |
| 14 | ++ |
| 15 | +++ |
| 16 | + |
| 18 | ++ |
| 19 | ++ |
| 20 | +++ |
| 22 | +++ |
| 23 | +++ |
| 24 | +++ |
| 29 | ++ |
| 30 | + |
| 31 | ++ |
| 32 | +++ |
| 33 | +++ |
| 34 | ++ |
| 35 | ++ |
| 36 | ++ |
| 37 | ++ |
| 39 | ++ |
| 40 | + |
| 41 | ++ |
| 42 | ++ |
| 43 | ++ |
| 44 | ++ |
| 45 | ++ |
| 46 | ++ |
| 48, sodium salt | ++ |
| 49 | ++ |
| 50, sodium salt | + |
| 51 | + |
| 52 | +++ |
| 53 | ++ |
| 54 | + |
| 56 | ++ |
| 57 | ++ |
| 59 | + |
| 60 | + |
| 61 | +++ |
| 62 | + |
| 63 | + |
| 64 | + |
| 65 | − |
| 66 | + |
| 67 | +++ |
| 68 Na salt | +++ |
| 70 | +++ |
| 71 | ++ |
| 72 Na salt | +++ |
| 75 | ++ |
| 76 | + |
| 77 | ++ |
| 78 | + |
| 79 | +++ |

¹Percent inhibition of compound tested at 100 μM.
+ = % inhibition < 30%
++ = 30 ≤ % inhibition ≤ 60%
+++ = % inhibition > 60%

### Example 81. Assessment of GO Inhibition in Cell Culture

Primary hepatocyte isolation. Hepatocytes are isolated in situ using the collagenase perfusion method from C57BL/6 Agxt1−/− mice liver. Mice are anesthetized with 20 mg/mL pentobarbital. Livers are perfused with 50 mL of buffer A (10 mM HEPES, 6.7 mM KCl, 145 mM NaCl, 2.4 mM EGTA, pH 7.4) and 50 mL of buffer B (100 mM HEPES, 6.7 mM KCl, 67 mM NaCl, 10 mg/mL albumin, 4.8 mM CaCl₂, 0.5 mg/mL collagenase, pH 7.4) at 37° C. and a constant influx rate of 8 mL/min. Livers are homogenized and filtered using a nylon sieve. Cells are washed three times with cold incomplete Williams E culture medium to inactivate the collagenase and centrifuged. Finally, cells are re-suspended in complete Williams E medium (2 mM L-glutamine, 100 U/mL penicillin, 100 μg/mL streptomycin, 2.2 mU/mL insulin and 0.3 μg/mL hydrocortisone).

Primary hepatocyte culture. 3.0×10⁵ cells/well are cultured in 6-well collagen-precoated plates with Williams E medium supplemented with 5% fetal bovine serum, 2 mM L-glutamine, 100 U/mL penicillin, 100 μg/mL streptomycin, 2.2 mU/mL insulin and 0.3 μg/mL hydrocortisone. After 6 hours, medium is changed to Williams E complete medium without serum and cells are treated with compounds of the invention in the presence of 5 mM glycolate. Culture medium is harvested for oxalate quantification at 24, 48, and 72 hours after treatment.

Cell viability and toxicity. Compound toxicity is evaluated by measurement of viable cells using the Cell Titer 96® Aqueous One Solution Reagent (Promega). Metabolically active cells reduce a tetrazolium compound (MTS) to form a soluble colored formazan product by dehydrogenase enzymes coupled to NADH or NADPH, and the absorbance of the formazan produced is directly proportional to the number of viable cells. 1.0×10⁴ cells/well are seeded in 96-well collagen-precoated plates and treated with the same concentrations of inhibitors as previously described for 6-well plates. At each time point (24, 48, and 72 hours), background absorbance is measured, followed by addition of MTS to the medium. Plates are incubated 2 h at 37° C., 5% CO₂, and measured at 493 nm. Significant cytotoxicity is not observed.

Quantification of oxalate is determined using a commercially available oxalate oxidase assay kit (Trinity Biotech). As shown in FIG. 2, oxalate is oxidized with molecular oxygen by the enzyme oxalate oxidase to produce carbon dioxide and hydrogen peroxide. Horseradish peroxidase catalyzes the reaction of the hydrogen peroxide with 3-methyl-2-benzothiazolinone hydrazone (MBTH) and 3-(dimethylamino) benzoic acid (DMAB) to form an indamine dye product. 100 μL of culture medium is harvested and frozen at −80° C. for lyophilization overnight. In a 96-well plate, standard curves are prepared by dilution of 0.5 mM oxalate in culture medium (0, 0.5, 1, 2, 4, 5 nmol oxalate). Samples and standards are then resuspended in 200 μL of solution A and 20 μL of solution B from the commercial kit, incubated at 37° C. for 15 minutes, and read for absorbance at 590 nm.

### VII. Exemplary Embodiments

Exemplary embodiments provided in accordance with the presently disclosed subject matter include, but are not limited to, the claims and the following embodiments:
1. A compound according to Formula I:

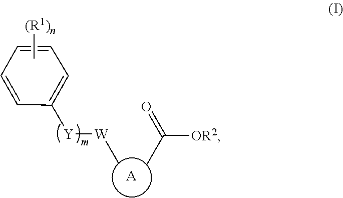

or a pharmaceutically acceptable salt or C₁₋₆ alkyl ester thereof,

wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₁₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₁₋₆ alkenylene, C₁₋₆ alkynylene, and 2- to 6-membered heteroalkylene; and
each heterocyclyl is optionally and independently substituted with one or more amine protecting groups;
provided that if ring A is (5-methyl)-isoxazol-3,4-diyl, W is —CH₂—, subscript n is 1, and subscript m is 1, then Y is 0;
provided that if ring A is (5-methyl)-isoxazol-3,4-diyl, W is —CH₂—, and subscript m is 0, then subscript n is 1, 2, or 3;
provided that if ring A is (5-methyl)-isoxazol-3,4-diyl, W is —CH₂—, subscript n is 1, and subscript m is 0, then R is other than 3-bromo, 4-bromo, 3-chloro, 4-chloro, 3-fluoro, and 4-fluoro;
provided that if ring A is (5-methyl)-1H-pyrazol-3,4-diyl, W is —NR³—, R³ is H or C₁₋₆ alkyl, and subscript m is 0, then subscript n is 1, 2, or 3;
provided that if ring A is (5-methyl)-1H-pyrazol-3,4-diyl, W is —NR³—, R³ is H or C₁₋₆ alkyl, subscript m is 1, and Y is —CH₂—, then subscript n is 1, 2, or 3;
provided that if ring A is (5-methyl)-isoxazol-3,4-diyl, W is —NH—, subscript n is 1, and subscript m is 0, then R is other than 3-cyano, 4-cyano, 3-bromo, 4-bromo, 3-chloro, 4-chloro, 3, fluoro, and 4-fluoro;
provided that if ring A is (5-methyl)-1H-pyrazol-3,4-diyl, W is —CH₂—, subscript n is 1, and subscript m is 1, then Y is 0;
provided that if ring A is (5-methyl)-1H-pyrazol-3,4-diyl, W is —CH₂—, and subscript m is 0, then subscript n is 1, 2, or 3;
provided that if ring A is (5-methyl)-1H-pyrazol-3,4-diyl, W is —CH₂—, subscript n is 1, and subscript m is 0, then R¹ is other than 3-cyano, 4-cyano, 4-bromo, 3-chloro, 4-chloro, 3-fluoro, 4-fluoro, 3-pyridin-3-yl, 3-pyridin-4-yl, 3-(4-cyanophenyl), 3-(4-fluorophenyl), 4-(4-fluorophenyl), 3-phenoxyphenyl, and 4-phenoxyphenyl;
provided that if ring A is 1,2,3-thiadiazol-4,5-diyl, W is —CH₂— or —NH—, and subscript m is 0, then subscript n is 1, 2, or 3;
provided that if ring A is 1,2,3-thiadiazol-4,5-diyl, W is —S—, Y is —CH₂—, and subscript m is 1, then subscript n is 1, 2, or 3;
provided that if ring A is 1,2,3-thiadiazol-4,5-diyl, W is S, subscript n is 1, and subscript m is 0, then R¹ is other than 4-chloro;
provided that if ring A is 1,2,3-thiadiazol-4,5-diyl, W is —NR³—, Y is —CHR⁴—, R³ is butyl, R⁴ is H, subscript n is 1, and subscript m is 1, then R¹ is other than 4-(2H-tetrazol-5-yl)phenyl;
provided that if ring A is pyridazin-3,4-diyl, W is —NR³—, Y is —CHR⁴—, R³ is propyl, R⁴ is H, subscript n is 1, and subscript m is 1, then R¹ is other than 4-(2H-tetrazol-5-yl)phenyl.
2. The compound of embodiment 1, or a pharmaceutically acceptable salt thereof, according to Formula III:

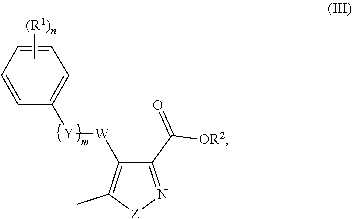

wherein Z is selected from the group consisting of NH, O, and S.
3. The compound of embodiment 1, or a pharmaceutically acceptable salt thereof, according to Formula IIIa:

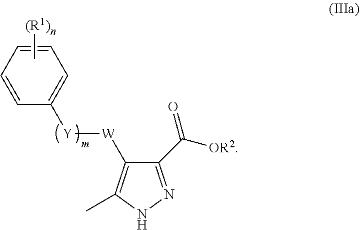

4. The compound of embodiment 1, or a pharmaceutically acceptable salt thereof, according to Formula IIIb:

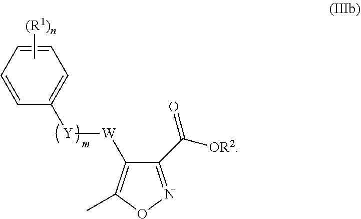

5. The compound of embodiment 1, or a pharmaceutically acceptable salt thereof, according to Formula IIIc:

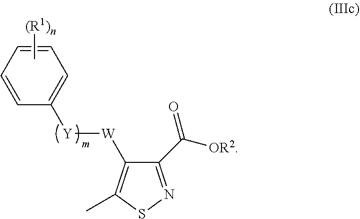

6. The compound of embodiment 1, or a pharmaceutically acceptable salt thereof, according to Formula II:

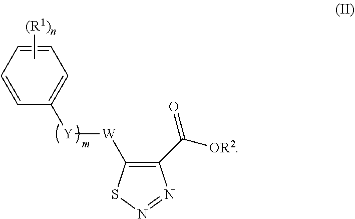

7. The compound of embodiment 1, or a pharmaceutically acceptable salt thereof, according to Formula IV:

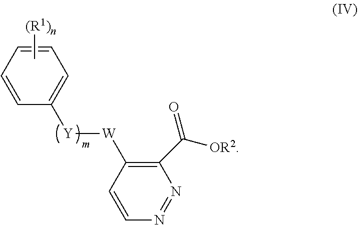

8. The compound of any one of embodiments 1-7, or a pharmaceutically acceptable salt thereof, wherein each Y is independently selected from the group consisting of —O— and —CHR⁴—.
9. The compound of any one of embodiments 1-8, or a pharmaceutically acceptable salt thereof, R^{3a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b}
10. The compound of any one of embodiments 1-9, or a pharmaceutically acceptable salt thereof, wherein L, M, and Q are independently selected from the group consisting of a bond, —O—, C₁₋₆ alkylene, and 2- to 6-membered heteroalkylene.
11. The compound of any one of embodiments 1-10, or a pharmaceutically acceptable salt thereof, wherein W is —NR³—.
12. The compound of embodiment 11, or a pharmaceutically acceptable salt thereof, wherein subscript m is 0.
13. The compound of embodiment 11 or embodiment 12, or a pharmaceutically acceptable salt thereof, wherein subscript n is 1 or 0.
14. The compound of any one of embodiments 11-13, or a pharmaceutically acceptable salt thereof, wherein subscript n is 1 and R¹ is selected from the group consisting of halo and cyano.
15. The compound of any one of embodiments 11-13, or a pharmaceutically acceptable salt thereof, wherein:
subscript n is 1;
R¹ is selected from the group consisting of heteroaryl and -L-(C₆₋₁₀ aryl), wherein aryl is substituted with R^{1a};
R^{1a} is selected from the group consisting of halo, cyano, and -M-heterocyclyl, heterocyclyl is optionally substituted with one or more amine protecting groups,
L is selected from the group consisting of a bond, —O—, and C₁₋₆ alkylene, and
M is 2- to 6-membered heteroalkylene.
16. The compound of any one of embodiments 11-15, or a pharmaceutically acceptable salt thereof, wherein R³ is H.
17. The compound of embodiment 11, or a pharmaceutically acceptable salt thereof, wherein subscript m is 1 or 2 and Y is —CHR⁴—.
18. The compound of embodiment 11 or embodiment 17, or a pharmaceutically acceptable salt thereof, wherein subscript n is 0 or 1.
19. The compound of any one of embodiments 11, 17, and 18, or a pharmaceutically acceptable salt thereof, wherein:
subscript n is 1
R¹ is halo;
R³ is C₇₋₁₆ arylalkyl substituted with R^{3a}; and
R^{3a} is halo.
20. The compound of any one of embodiments 11, 17, and 18, or a pharmaceutically acceptable salt thereof, wherein:
subscript n is 1;
R¹ is selected from the group consisting of halo, cyano, and -L-(C₆₋₁₀ aryl), wherein aryl is optionally substituted with R^{1a};
R^{1a} is selected from the group consisting of halo and cyano;
L is selected from the group consisting of a bond, —O—, and C₁₋₆ alkylene; and
R³ is H.
21. The compound of any one of embodiments 11 and 17-20, or a pharmaceutically acceptable salt thereof, wherein each R⁴ is H.
22. The compound of any one of embodiments 1-10, or a pharmaceutically acceptable salt thereof, wherein:
W is —NR³—;
Y is —CHR⁴—;
subscript n is 0 or 1;
subscript m is 0, 1, or 2;
R¹ is halo or -L-(C₆₋₁₀ aryl), wherein aryl is optionally substituted with R^{1a};
R³ is C₇₋₁₆ arylalkyl, wherein aryl in arylalkyl is substituted with R^{3a};
R⁴ is H;
R^{3a} is halo or -M-(C₆₋₁₀ aryl), wherein aryl is optionally substituted with R^{3b};
each of R^{1b} and R^{3b} is independently selected from the group consisting of halo and cyano;
L and M are independently selected from the group consisting of a bond, —O—, C₁₋₆ alkylene, and 2- to 6-membered heteroalkylene; and
each heterocyclyl is optionally and independently substituted with one or more amine protecting groups.
23. The compound of embodiment 22, wherein subscript n is 1.
24. The compound of any one of embodiments 1-10, or a pharmaceutically acceptable salt thereof, wherein W is —CHR³—.
25. The compound of embodiment 24, or a pharmaceutically acceptable salt thereof, wherein W is —CH₂—.
26. The compound of embodiment 24 or embodiment 25, or a pharmaceutically acceptable salt thereof, wherein subscript m is 0.
27. The compound of embodiment 24 or embodiment 25, or a pharmaceutically acceptable salt thereof, wherein subscript m is 1 and Y is —O—.
28. The compound of any one of embodiments 24-27, or a pharmaceutically acceptable salt thereof, wherein subscript n is 0 or 1.
29. The compound of any one of embodiments 24-28, or a pharmaceutically acceptable salt thereof, wherein:
subscript n is 1;
R¹ is selected from the group consisting of halo and -L-aryl,
L is selected from the group consisting of a bond and —O—, and
aryl is optionally substituted with R^{1a}
30. The compound of any one of embodiments 1-10, or a pharmaceutically acceptable salt thereof, wherein W is selected from the group consisting of —S—, —S(O)—, and —S(O)₂—.
31. The compound of embodiment 30, or a pharmaceutically acceptable salt thereof, wherein subscript m is 0.
32. The compound of embodiment 30, or a pharmaceutically acceptable salt thereof, wherein subscript m is 1 and Y is —CHR⁴—.
33. The compound of any one of embodiments 30-32, or a pharmaceutically acceptable salt thereof, wherein subscript n is 0 or 1.
34. The compound of embodiment 32, wherein R⁴ is C₁₋₆ alkyl.
35. The compound of embodiment 32, wherein R⁴ is C₇₋₁₆ arylalkyl, wherein aryl in arylalkyl is optionally substituted with R^{4a}
36. The compound of any one of embodiments 30-35, or a pharmaceutically acceptable salt thereof, wherein subscript n is 0 or 1.
37. The compound of any one of embodiments 30-35, or a pharmaceutically acceptable salt thereof, wherein subscript n is 0.
38. The compound of any one of embodiments 30-35, or a pharmaceutically acceptable salt thereof, wherein subscript n is 1 and R¹ is halo.
39. The compound of any one of embodiments 1-38, wherein R² is H.
40. The compound of any one of embodiments 1-38, wherein R² is C₁₋₆ alkyl.
41. The compound of embodiment 1, which is selected from the group consisting of

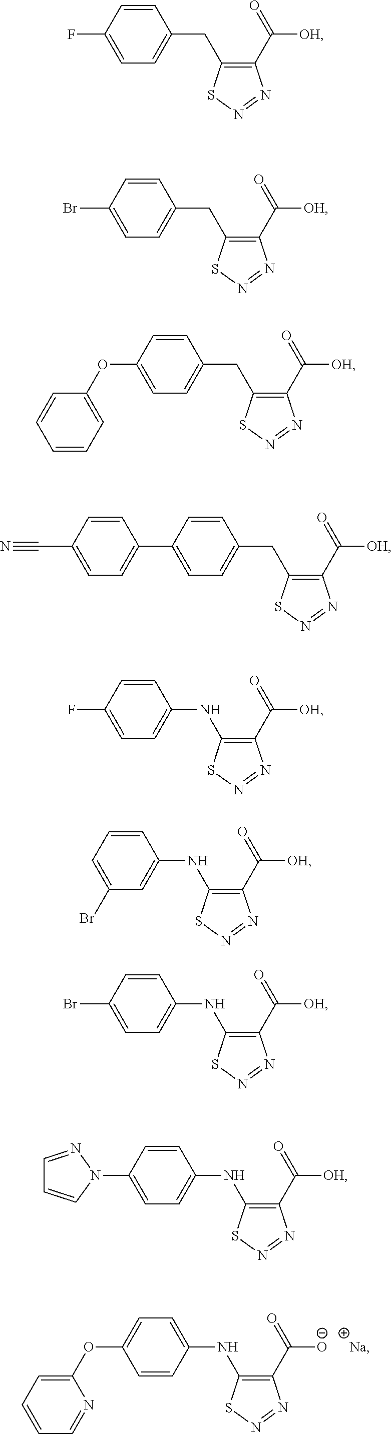

and pharmaceutically acceptable salts thereof.
42. The compound of embodiment 1, which is selected from the group consisting of

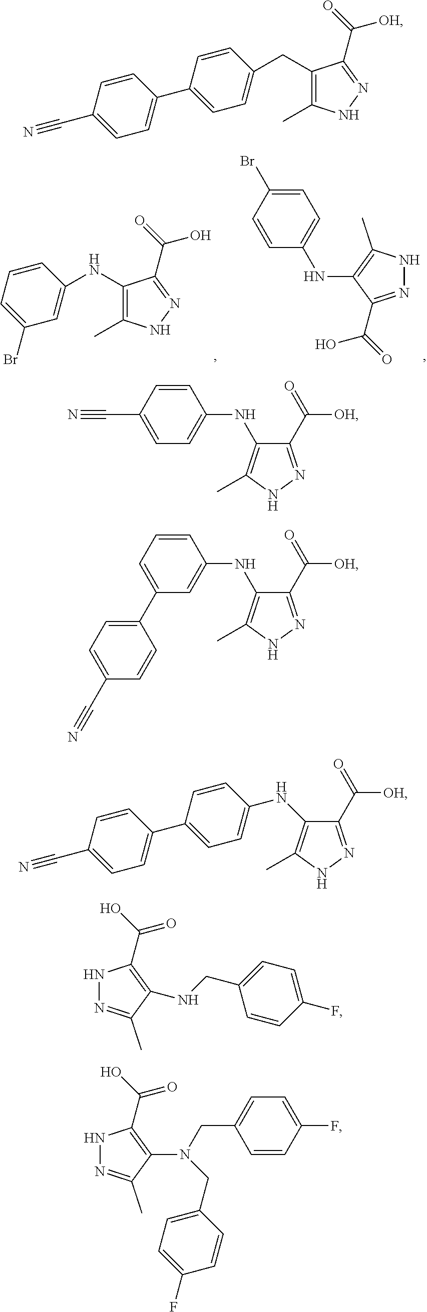

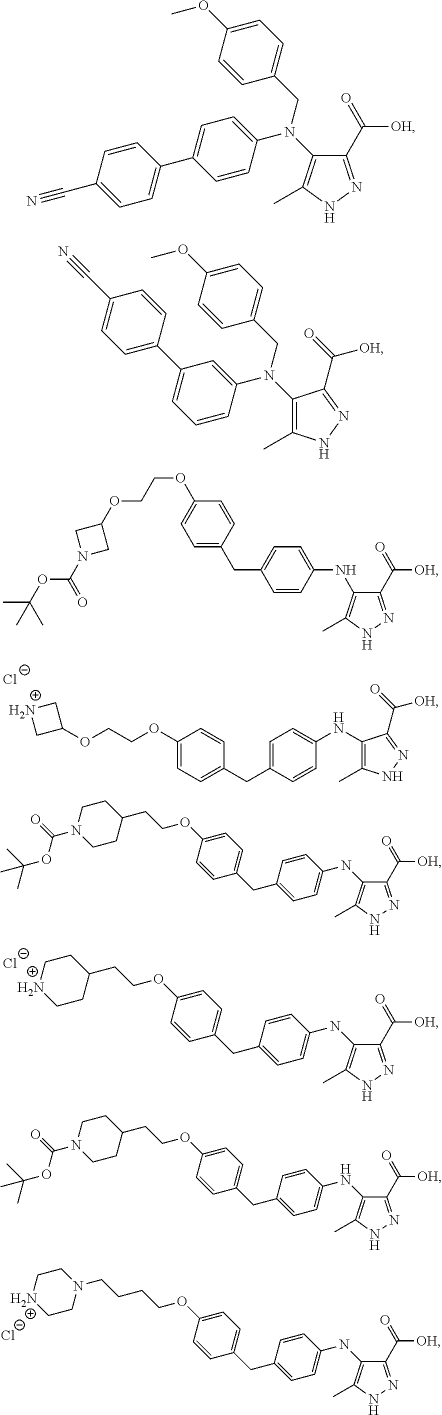

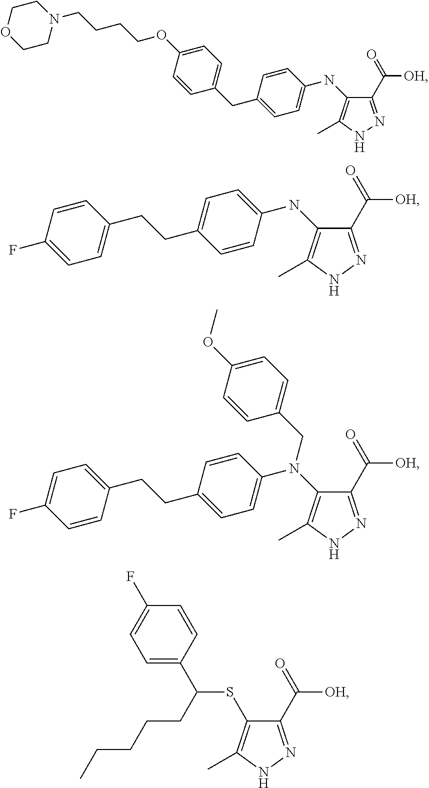

and pharmaceutically acceptable salts thereof.
43. The compound of embodiment 1, which is selected from the group consisting of

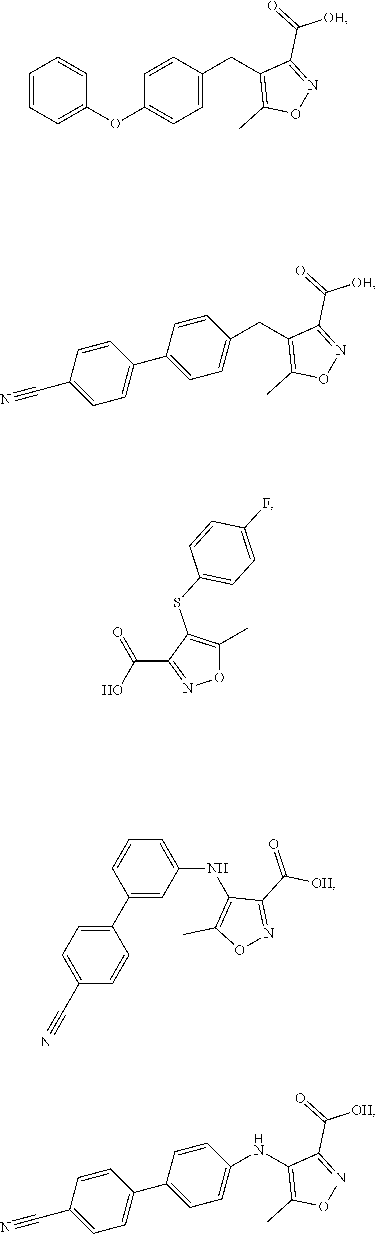 [SMILES: Cc1onc(C(=O)O)c1Cc1ccc(Oc2ccccc2)cc1 ; Cc1onc(C(=O)O)c1Cc1ccc(-c2ccc(C#N)cc2)cc1 ; Cc1onc(C(=O)O)c1Sc1ccc(F)cc1 ; Cc1onc(C(=O)O)c1Nc1cccc(-c2ccc(C#N)cc2)c1 ; Cc1onc(C(=O)O)c1Nc1ccc(-c2ccc(C#N)cc2)cc1]

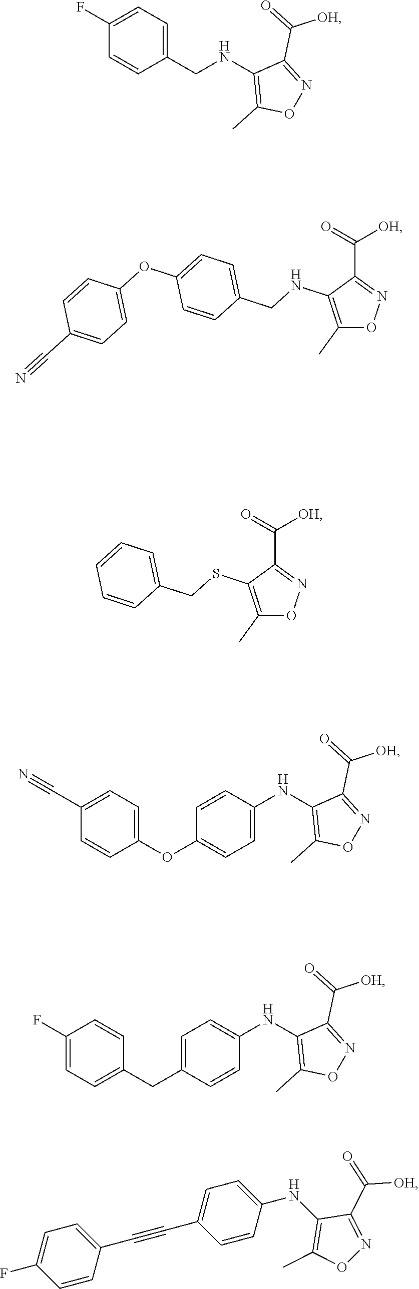 [SMILES: Cc1onc(C(=O)O)c1NCc1ccc(F)cc1 ; Cc1onc(C(=O)O)c1NCc1ccc(Oc2ccc(C#N)cc2)cc1 ; Cc1onc(C(=O)O)c1SCc1ccccc1 ; Cc1onc(C(=O)O)c1Nc1ccc(Oc2ccc(C#N)cc2)cc1 ; Cc1onc(C(=O)O)c1Nc1ccc(Cc2ccc(F)cc2)cc1 ; Cc1onc(C(=O)O)c1Nc1ccc(C#Cc2ccc(F)cc2)cc1]

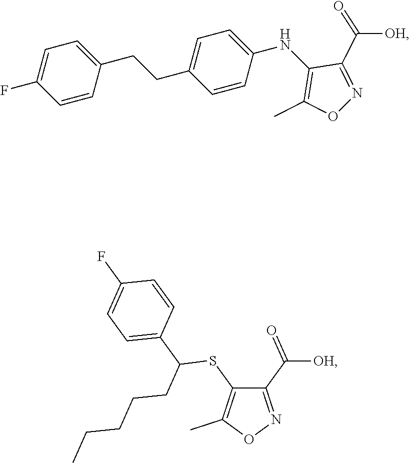 [SMILES: Cc1onc(C(=O)O)c1Nc1ccc(CCc2ccc(F)cc2)cc1 ; CCCCCC(Sc1c(C(=O)O)noc1C)c1ccc(F)cc1]

and pharmaceutically acceptable salts thereof.
44. The compound of embodiment 1, which is selected from the group consisting of

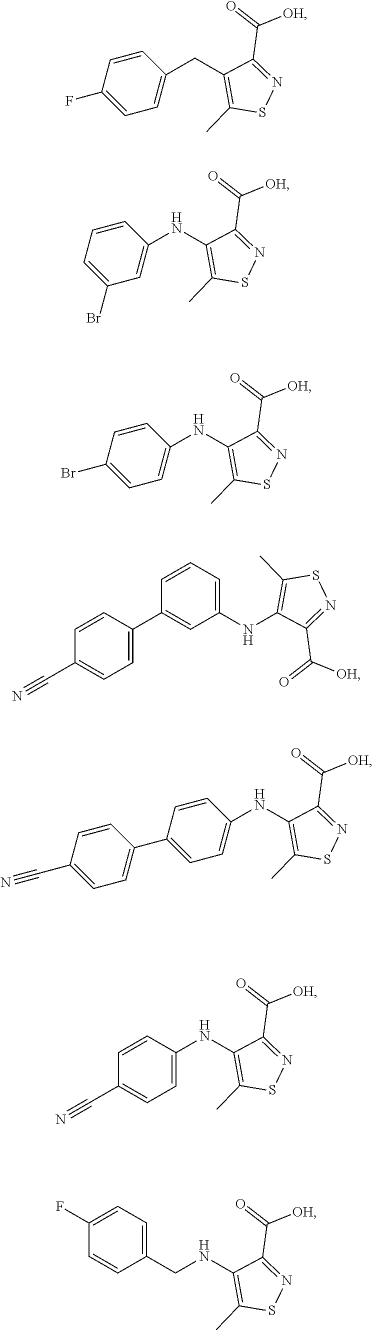

and pharmaceutically acceptable salts thereof.
45. The compound of embodiment 1, which is selected from the group consisting of

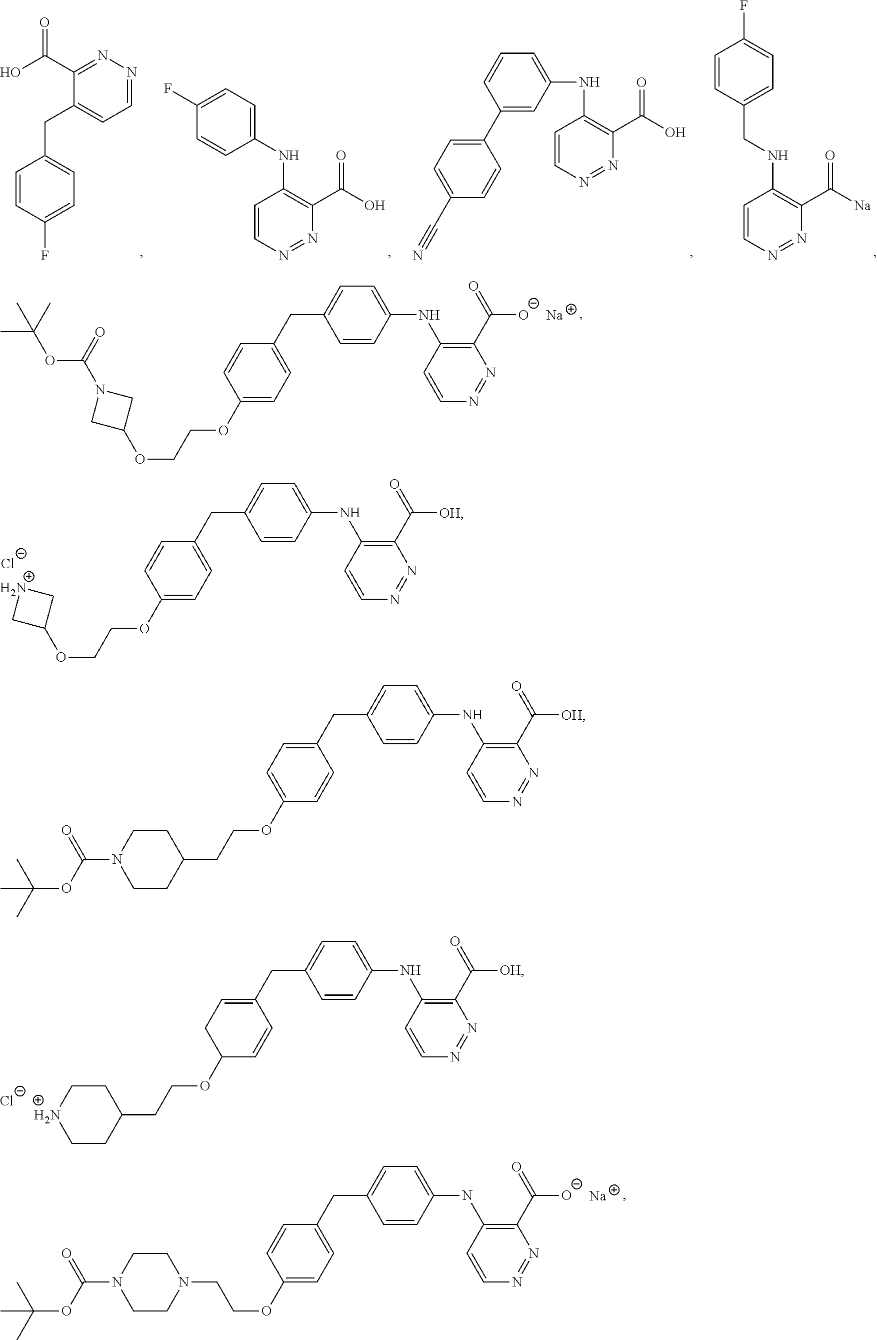

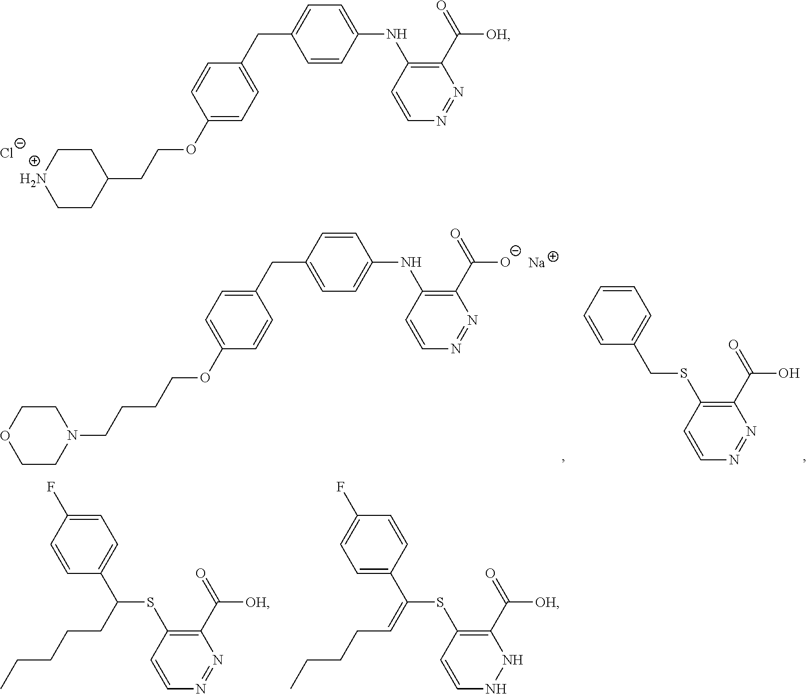

and pharmaceutically acceptable salts thereof.
46. A pharmaceutical composition comprising a compound according to any one of embodiments 1-45 and a pharmaceutically acceptable excipient.
47. A method for treating primary hyperoxaluria, type I (PH1) comprising administering to a subject in need thereof a therapeutically effective amount of a compound according to Formula I:

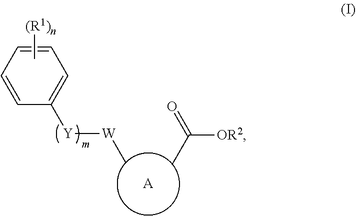

or a pharmaceutically acceptable salt thereof, wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₁₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₁₋₆ alkenylene, C₁₋₆ alkynylene, and 2- to 6-membered heteroalkylene; and
each heterocyclyl is optionally and independently substituted with one or more amine protecting groups;
provided that if ring A is 1,2,3-thiadiazol-4,5-diyl, W is S, subscript n is 1, and subscript m is 0, then R¹ is other than 4-chloro;
thereby treating the PH1.
48. A method for treating kidney stones comprising administering to a subject in need thereof a therapeutically effective amount of a compound according to Formula I:

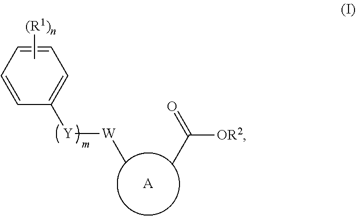

or a pharmaceutically acceptable salt thereof, wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₁₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₁₋₆ alkenylene, C₁₋₆ alkynylene, and 2- to 6-membered heteroalkylene; and
each heterocyclyl is optionally and independently substituted with one or more amine protecting groups;
provided that if ring A is 1,2,3-thiadiazol-4,5-diyl, W is S, subscript n is 1, and subscript m is 0, then R¹ is other than 4-chloro;
thereby treating the kidney stones.
49. The method of embodiment 47 or embodiment 48, wherein each Y is independently selected from the group consisting of —O— and —CHR⁴—.
50. The method of any one of embodiments 47-49, wherein R³ is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b}
51. The method of any one of embodiments 47-50, wherein L, M, and Q are independently selected from the group consisting of a bond, —O—, C₁₋₆ alkylene, and 2- to 6-membered heteroalkylene.
52. The method of any one of embodiments 47-51, wherein subscript n is 0 or 1.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A compound according to Formula I: 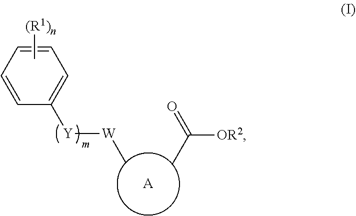
or a pharmaceutically acceptable salt or C₁₋₆ alkyl ester thereof,
wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 1, 2, 3, or 0;
subscript m is 0, 1, or 2;
W is selected from the group consisting of —NR³—, —C(R³)₂—, —O—, —S—, —S(O)—, and —S(O)₂—;
each Y, if present, is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₂₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, C₁₋₆ alkylene, C₂₋₆ alkenylene, C₂₋₆ alkynylene, and 2- to 6-membered heteroalkylene; and
each heterocyclyl is optionally and independently substituted with one or more amine protecting groups;
provided that if ring A is 1,2,3-thiadiazol-4,5-diyl, W is —NR³—, R³ is butyl, subscript m is 1, Y is —CHR⁴—, R⁴ is H, and subscript n is 1, then R¹ is other than 4-(2H-tetrazol-5-yl)phenyl;
provided that if ring A is 1,2,3-thiadiazol-4,5-diyl, W is —CH₂— or —NH—, and subscript m is 0, then subscript n is 1, 2, or 3;
provided that if ring A is 1,2,3-thiadiazol-4,5-diyl, W is —S—, subscript m is 1, and Y is —CH₂—, then subscript n is 1, 2, or 3;
provided that if ring A is 1,2,3-thiadiazol-4,5-diyl, W is S, subscript n is 1, and subscript m is 0, then R¹ is other than 4-chloro;
provided that if ring A is (5-methyl)-isoxazol-3,4-diyl, W is —CH₂—, subscript n is 1, and subscript m is 1, then Y is 0;
provided that if ring A is (5-methyl)-isoxazol-3,4-diyl, W is —CH₂—, and subscript m is 0, then subscript n is 1, 2, or 3;
provided that if ring A is (5-methyl)-isoxazol-3,4-diyl, W is —CH₂—, subscript n is 1, and subscript m is 0, then R¹ is other than 3-bromo, 4-bromo, 3-chloro, 4-chloro, 3-fluoro, and 4-fluoro;
provided that if ring A is (5-methyl)-1H-pyrazol-3,4-diyl, W is —NR³—, R³ is H or C₁₋₆ alkyl, and subscript m is 0, then subscript n is 1, 2, or 3;
provided that if ring A is (5-methyl)-1H-pyrazol-3,4-diyl, W is —NR³—, R³ is H or C₁₋₆ alkyl, subscript m is 1, and Y is —CH₂—, then subscript n is 1, 2, or 3;
provided that if ring A is (5-methyl)-isoxazol-3,4-diyl, W is —NH—, subscript n is 1, and subscript m is 0, then R¹ is other than 3-cyano, 4-cyano, 3-bromo, 4-bromo, 3-chloro, 4-chloro, 3, fluoro, and 4-fluoro;
provided that if ring A is (5-methyl)-1H-pyrazol-3,4-diyl, W is —CH₂—, subscript n is 1, and subscript m is 1, then Y is 0;
provided that if ring A is (5-methyl)-1H-pyrazol-3,4-diyl, W is —CH₂—, and subscript m is 0, then subscript n is 1, 2, or 3;
provided that if ring A is (5-methyl)-1H-pyrazol-3,4-diyl, W is —CH₂—, subscript n is 1, and subscript m is 0, then R¹ is other than 3-cyano, 4-cyano, 4-bromo, 3-chloro, 4-chloro, 3-fluoro, 4-fluoro, 3-pyridin-3-yl, 3-pyridin-4-yl, 3-(4-cyanophenyl), 3-(4-fluorophenyl), 4-(4-fluorophenyl), 3-phenoxyphenyl, and 4-phenoxyphenyl;
provided that if ring A is pyridazin-3,4-diyl, W is —NR³—, Y is —CHR⁴—, R³ is propyl, R⁴ is H, subscript n is 1, and subscript m is 1, then R¹ is other than 4-(2H-tetrazol-5-yl)phenyl.

2. The compound of claim 1, or a pharmaceutically acceptable salt thereof, according to a formula selected from the group consisting of Formula II, Formula III, and Formula IV: 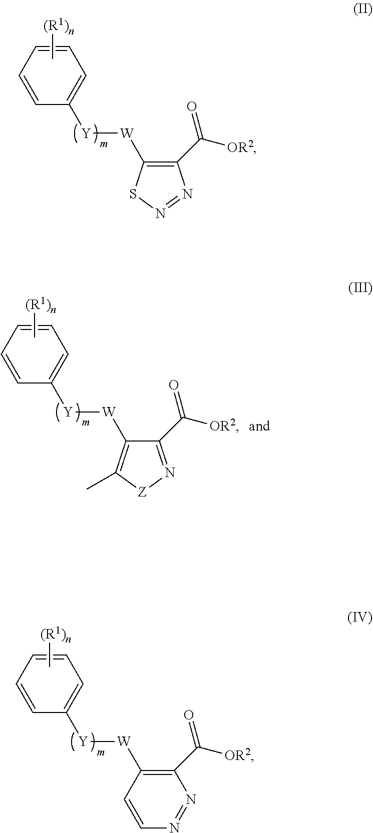
wherein Z is selected from the group consisting of NH, O, and S.

3. (canceled)

4. The compound of claim 1, or a pharmaceutically acceptable salt thereof, according to a formula selected from the group consisting of Formula IIIa, Formula IIIb, and Formula IIIc:

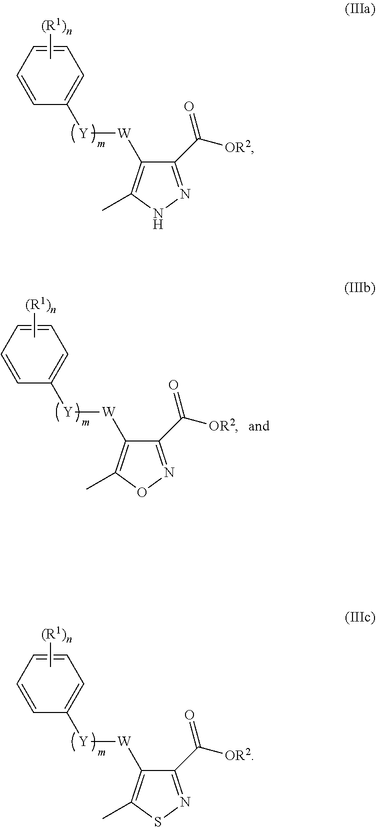

5-7. (canceled)

8. The compound of claim 1, or a pharmaceutically acceptable salt thereof, wherein W is —NR³—.

9-13. (canceled)

14. The compound of claim 8, or a pharmaceutically acceptable salt thereof, wherein:
subscript n is 1;
R¹ is selected from the group consisting of heteroaryl and -L-(C₆₋₁₀ aryl), wherein aryl is substituted with R^{1a};
R^{1a} is selected from the group consisting of halo, cyano, and -M-heterocyclyl, heterocyclyl is optionally substituted with one or more amine protecting groups,
L is selected from the group consisting of a bond, —O—, and C₁₋₆ alkylene, and
M is 2- to 6-membered heteroalkylene.

15-17. (canceled)

18. The compound of claim 8, or a pharmaceutically acceptable salt thereof, wherein:
subscript n is 1
R¹ is halo;
R³ is C₇₋₁₆ arylalkyl substituted with R^{3a}; and
R^{3a} is halo.

19-20. (canceled)

21. The compound of claim 1, or a pharmaceutically acceptable salt thereof, wherein each Y is independently selected from the group consisting of —O— and —CHR⁴—.

22. The compound of claim 1, or a pharmaceutically acceptable salt thereof, R^{3a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b}.

23. The compound of claim 1, or a pharmaceutically acceptable salt thereof, wherein L, M, and Q are independently selected from the group consisting of a bond, —O—, C₁₋₆ alkylene, and 2- to 6-membered heteroalkylene.

24. The compound of claim 1, or a pharmaceutically acceptable salt thereof, wherein:
W is —NR³—;
Y is —CHR⁴—;
subscript n is 0 or 1;
subscript m is 0, 1, or 2;
R¹ is halo or -L-(C₆₋₁₀ aryl), wherein aryl is optionally substituted with R^{1a};
R³ is C₇₋₁₆ arylalkyl, wherein aryl in arylalkyl is substituted with R^{3a};
R⁴ is H;
R^{3a} is halo or -M-(C₆₋₁₀ aryl), wherein aryl is optionally substituted with R^{3b};
each of R^{1b} and R^{3b} is independently selected from the group consisting of halo and cyano;
L and M are independently selected from the group consisting of a bond, —O—, C₁₋₆ alkylene, and 2- to 6-membered heteroalkylene; and
each heterocyclyl is optionally and independently substituted with one or more amine protecting groups.

25. (canceled)

26. The compound of claim 1, or a pharmaceutically acceptable salt thereof, wherein W is —CHR³—.

27-31. (canceled)

32. The compound of claim 1, or a pharmaceutically acceptable salt thereof, wherein W is selected from the group consisting of —S—, —S(O)—, and —S(O)₂—.

33-42. (canceled)

43. The compound of claim 1, which is selected from the group consisting of 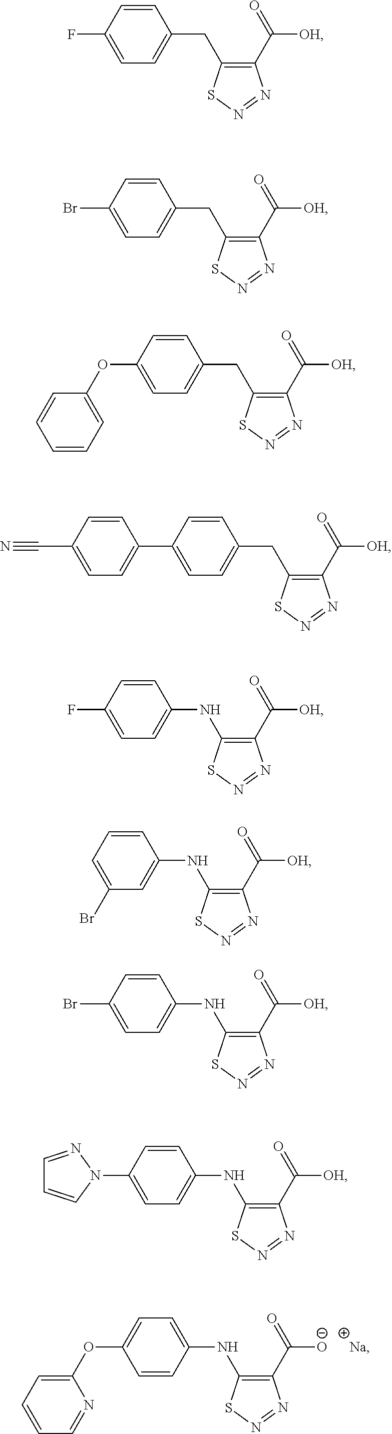
and pharmaceutically acceptable salts thereof.

44. The compound of claim 1, which is selected from the group consisting of 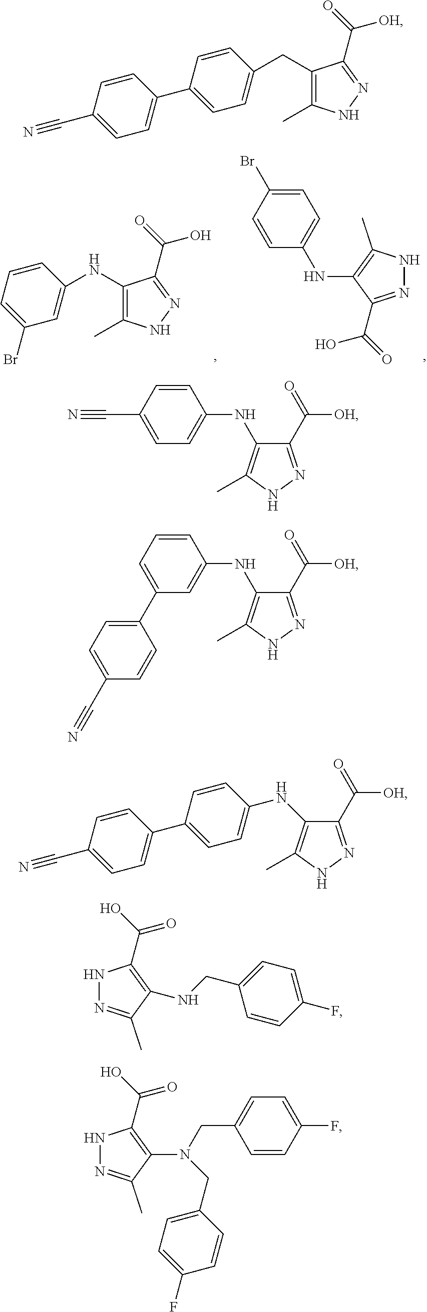 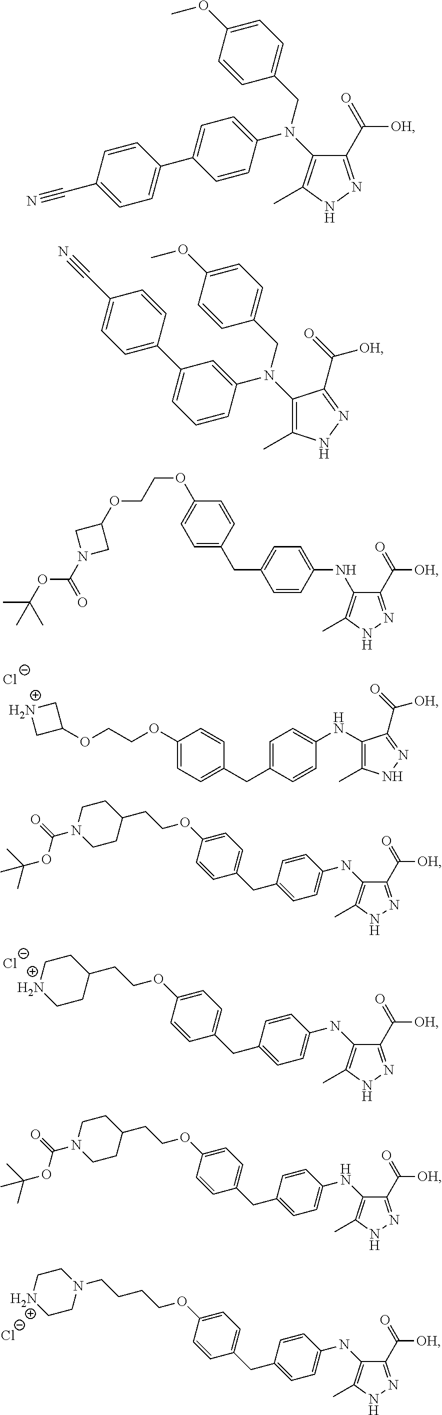 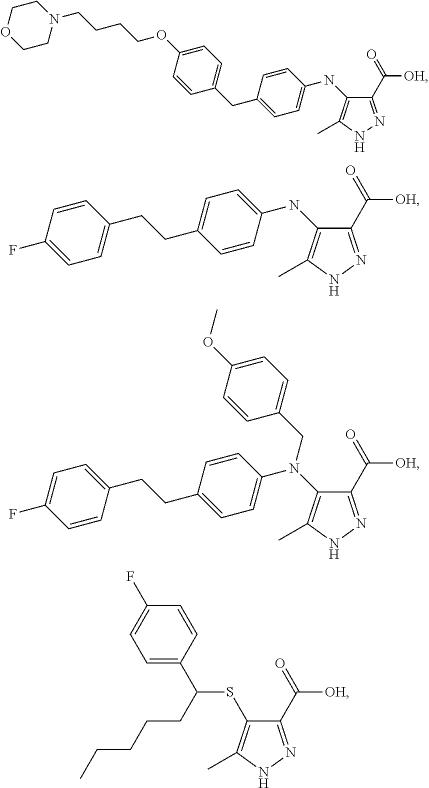
and pharmaceutically acceptable salts thereof.

45. The compound of claim 1, which is selected from the group consisting of 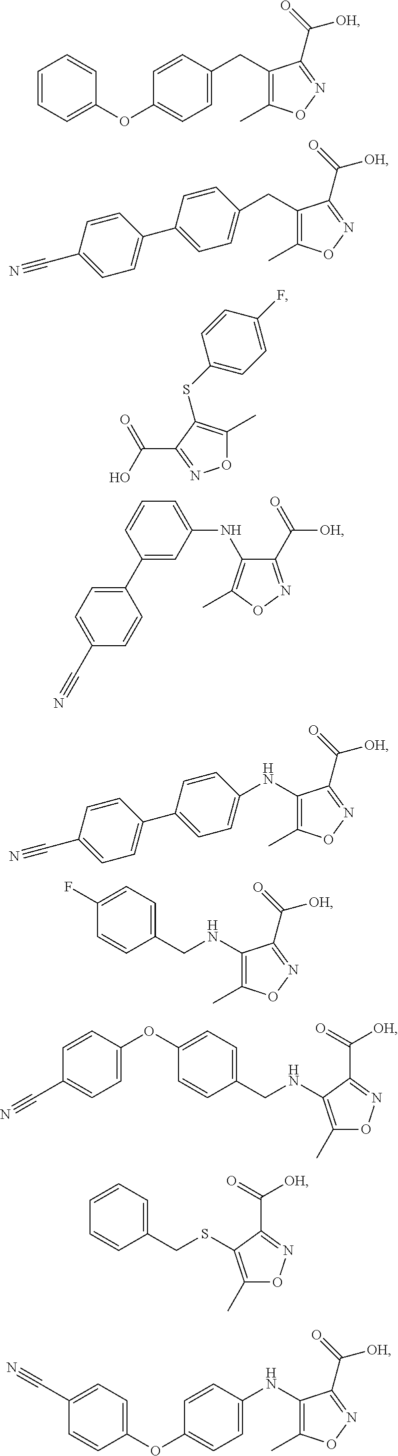 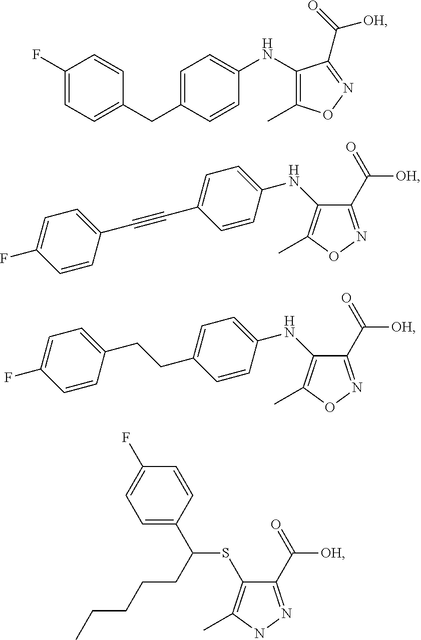
and pharmaceutically acceptable salts thereof.

46. The compound of claim 1, which is selected from the group consisting of 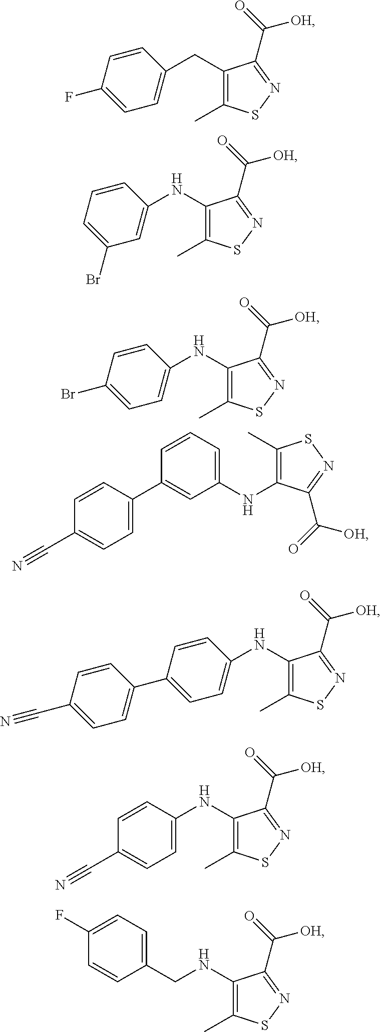
and pharmaceutically acceptable salts thereof.

47. The compound of claim 1, which is selected from the group consisting of 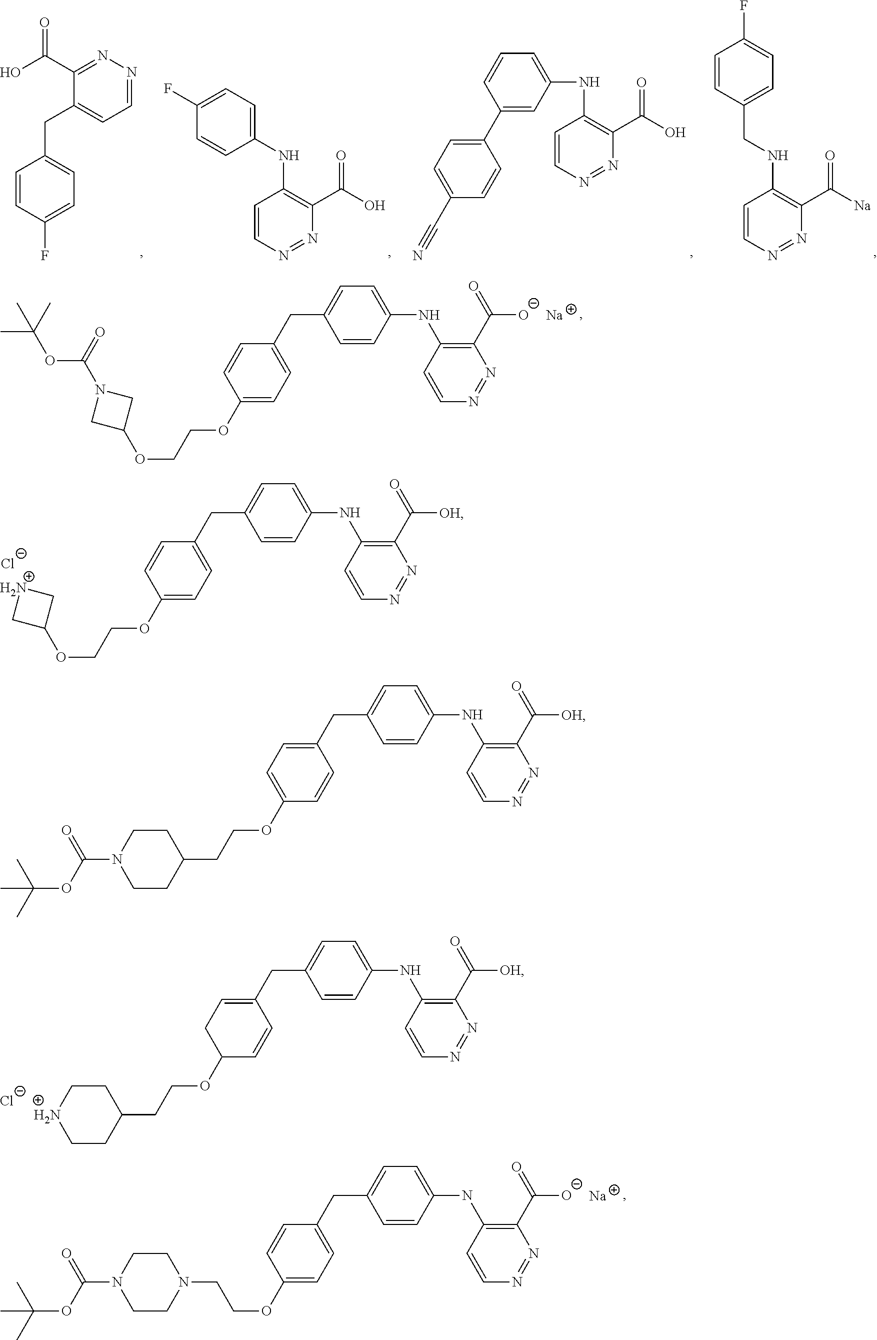 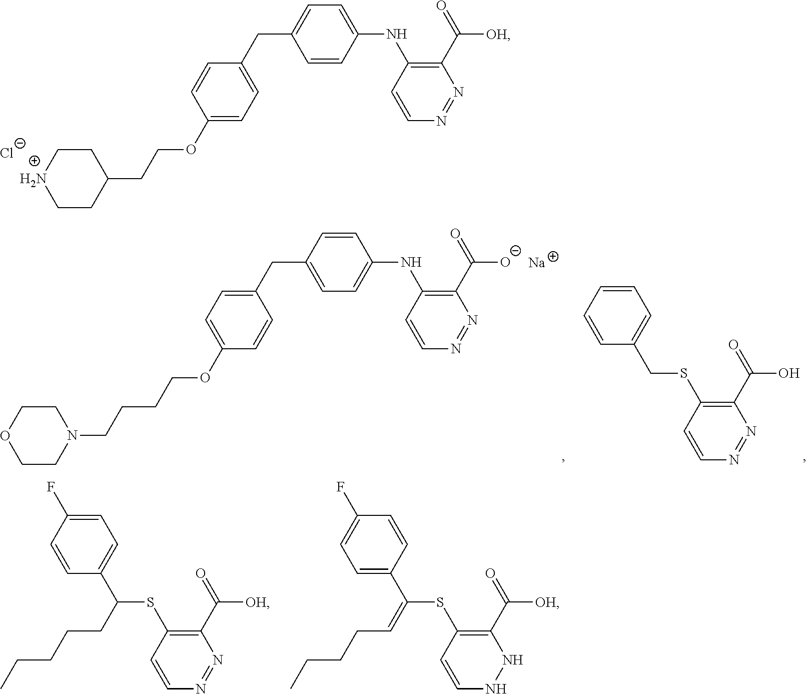
and pharmaceutically acceptable salts thereof.

48. A pharmaceutical composition comprising a compound according to claim 1 and a pharmaceutically acceptable excipient.

49. A method for treating primary hyperoxaluria, type I (PH1) comprising administering to a subject in need thereof a therapeutically effective amount of a compound according to Formula I: 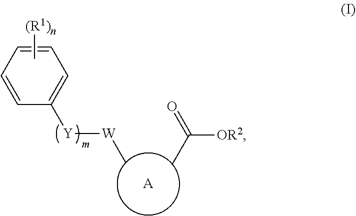
or a pharmaceutically acceptable salt thereof, wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₂₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R¹, R^{3b}, and R^{4b} is independently selected from the group consisting of halo and cyano;
L, M, and Q are independently selected from the group consisting of a bond, —O—, 37 C₁₋₆ alkylene, C₂₋₆ alkenylene, C₂₋₆ alkynylene, and 2- to 6-membered heteroalkylene; and
each heterocyclyl is optionally and independently substituted with one or more amine protecting groups;
provided that if ring A is 1,2,3-thiadiazol-4,5-diyl, W is S, subscript n is 1, and subscript m is 0, then R¹ is other than 4-chloro;
thereby treating the PH1.

50. A method for treating kidney stones comprising administering to a subject in need thereof a therapeutically effective amount of a compound according to Formula I: 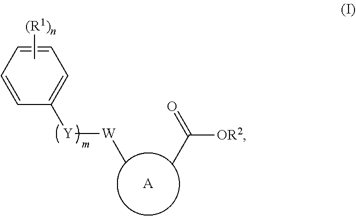
or a pharmaceutically acceptable salt thereof, wherein:
ring A is selected from the group consisting of 1,2,3-thiadiazol-4,5-diyl, (5-methyl)-1H-pyrazol-3,4-diyl, (5-methyl)-isoxazol-3,4-diyl, (5-methyl)-isothiazol-3,4-diyl, and pyridazin-3,4-diyl;
subscript n is 0, 1, 2, or 3;
subscript m is 0, 1, or 2;
W is selected from the group consisting of —NR³—, —C(R³)₂—, —O—, —S—, —S(O)—, and —S(O)₂—;
each Y is independently selected from the group consisting of —O— and —C(R⁴)₂—;
R¹ is selected from the group consisting of halo, cyano, 5- to 6-membered heteroaryl, and -L-(C₆₋₁₀ aryl), wherein aryl is optionally substituted with R^{1a};
R² is selected from the group consisting of H and C₁₋₆ alkyl;
each R³ is independently selected from the group consisting of H, C₁₋₆ alkyl, and C₇₋₁₆ arylalkyl, wherein aryl in arylalkyl is optionally substituted with R^{3a};
each R⁴ is independently selected from the group consisting of H, C₁₋₆ alkyl, and C₇₋₁₆ arylalkyl, wherein aryl in arylalkyl is optionally substituted with R^{4a}; or
two R⁴ are taken together to form C₂₋₆ alkenyl;
R^{1a} is independently selected from the group consisting of halo, cyano, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{1b};
R^{3a} is independently selected from the group consisting of halo, cyano, C₁₋₆ alkyl, C₁₋₆ alkoxy, -M-(8- to 12-membered heterocyclyl), -M-(5- to 6-membered heteroaryl), -M-(C₃₋₈ cycloalkyl), and -M-(C₆₋₁₀ aryl), wherein heterocyclyl, heteroaryl, cycloalkyl, and aryl are optionally substituted with R^{3b};
R^{4a} is independently selected from the group consisting of halo, cyano, -Q-(8- to 12-membered heterocyclyl), -Q-(5- to 6-membered heteroaryl), -Q-(C₃₋₈ cycloalkyl), and -Q-(C₆₋₁₀ aryl), wherein heterocyclyl, heteroaryl, cycloalkyl, and aryl are optionally substituted with R^{4b};
each of R^{1b}, R^{3b}, and R^{4b} is independently selected from the group consisting of halo and cyano;
L, M, and Q are independently selected from the group consisting of a bond, —O—, 37 C₁₋₆ alkylene, C₂₋₆ alkenylene, C₂₋₆ alkynylene, and 2- to 6-membered heteroalkylene; and
each heterocyclyl is optionally and independently substituted with one or more amine protecting groups;
provided that if ring A is 1,2,3-thiadiazol-4,5-diyl, W is S, subscript n is 1, and subscript m is 0, then R¹ is other than 4-chloro;
thereby treating the kidney stones.

51-54. (canceled)
